(19) United States
(12) Patent Application Publication
(10) Pub. No.: US 2021/0253528 A1
(43) Pub. Date: Aug. 19, 2021

(54) CHEMICAL COMPOUNDS

(71) Applicant: GLAXOSMITHKLINE INTELLECTUAL PROPERTY DEVELOPMENT LIMITED, Brentford, Middlesex (GB)

(72) Inventors: Michael P. DEMARTINO, Collegeville, PA (US); Karen Anderson EVANS, Collegeville, PA (US); Israil PENDRAK, Collegeville, PA (US); Joseph E. PERO, Collegeville, PA (US)

(21) Appl. No.: 16/973,587

(22) PCT Filed: Jul. 8, 2019
(86) PCT No.: PCT/IB2019/055811

§ 371 Date: Dec. 9, 2020

Related U.S. Application Data
(60) Provisional application No. 62/695,384, filed on Jul. 9, 2018.

(51) Int. Cl. C07D 213/64; C07C 271/28; C07C 275/30; C07C 235/20; C07D 207/273; A61K 45/06; A61K 31/506

(52) U.S. Cl. CPC C07D 213/64; C07C 271/28; C07C 275/30; A61K 31/506; C07D 207/273; A61K 45/06; C07C 235/20

(57) ABSTRACT

The invention is directed to substituted carbon-linked bicycloalkane derivatives. Specifically, the invention is directed to compounds according to Formula (X):

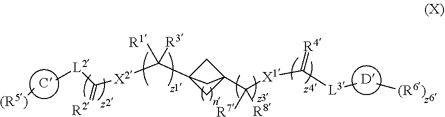

wherein C′, D′, L^{2′}, L^{3′}, R^{2′}, R^{3′}, R^{4′}, R^{5′}, R^{6′}, R^{7′}, R^{8′}, z²′, z³′, z⁴′, z⁵′, z⁶′, X¹, and X² are as defined herein; or a salt thereof including a pharmaceutically acceptable salt thereof. The compounds of the invention are inhibitors of the ATF4 pathway and can be useful in the treatment of cancer, pre-cancerous syndromes and diseases associated with activated unfolded protein response pathways, such as Alzheimer's disease, spinal cord injury, traumatic brain injury, ischemic stroke, stroke, diabetes, Parkinson disease, Huntington's disease, Creutzfeldt-Jakob Disease, and related prion diseases, progressive supranuclear palsy, amyotrophic lateral sclerosis, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementia, cognitive impairment, atherosclerosis, ocular diseases, neurological disorders, pain, arrhythmias, in organ transplantation and in the transportation of organs for transplantation. Accordingly, the invention is further directed to pharmaceutical compositions comprising a compound of the invention. The invention is still further directed to methods of inhibiting the ATF4 pathway and treatment of disorders associated therewith using a compound of the invention or a pharmaceutical composition comprising a compound of the invention.

### FIELD OF THE INVENTION

The present invention relates to substituted carbon-linked bicycloalkane derivatives that are inhibitors of the ATF4 pathway. The present invention also relates to pharmaceutical compositions comprising such compounds and methods of using such compounds in the treatment of diseases/injuries associated with activated unfolded protein response pathways, such as cancer, pre-cancerous syndromes, Alzheimer's disease, spinal cord injury, traumatic brain injury, ischemic stroke, stroke, diabetes, Parkinson disease, Huntington's disease, Creutzfeldt-Jakob Disease, and related prion diseases, progressive supranuclear palsy, amyotrophic lateral sclerosis, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementia, cognitive impairment, atherosclerosis, ocular diseases, neurological disorders, pain, arrhythmias, in organ transplantation and in the transportation of organs for transplantation.

### BACKGROUND OF THE INVENTION

In metazoa, diverse stress signals converge at a single phosphorylation event at serine 51 of a common effector, the translation initiation factor eIF2α. This step is carried out by four eIF2α kinases in mammalian cells: PERK, which responds to an accumulation of unfolded proteins in the endoplasmic reticulum (ER), GCN2 to amino acid starvation and UV light, PKR to viral infection, and HRI to heme deficiency. This collection of signaling pathways has been termed the “integrated stress response” (ISR), as they converge on the same molecular event. eIF2α phosphorylation results in an attenuation of translation with consequences that allow cells to cope with the varied stresses (1).

eIF2 (which is comprised of three subunits, α, β, and γ) binds GTP and the initiator Met-tRNA to form the ternary complex (eIF2-GTP-Met-tRNAD, which, in turn, associates with the 40S ribosomal subunit scanning the 5′UTR ofmRNAs to select the initiating AUG codon. Upon phosphorylation of its a-subunit, eIF2 becomes a competitive inhibitor of its GTP-exchange factor (GEF), eIF2B (2). The tight and nonproductive binding of phosphorylated eIF2 to eIF2B prevents loading of the eIF2 complex with GTP thus blocking ternary complex formation and reducing translation initiation (3). Because eIF2B is less abundant than eIF2, phosphorylation of only a small fraction of the total eIF2 has a dramatic impact on eIF2B activity in cells.

Paradoxically, under conditions of reduced protein synthesis, a small group of mRNAs that contain upstream open reading frames (uORFs) in their 5′UTR are translationally up-regulated (4,5). These include mammalian ATF4 (a cAMP element binding (CREB) transcription factor) and CHOP (a pro-apoptotic transcription factor) (6-8). ATF4 regulates the expression of many genes involved in metabolism and nutrient uptake and additional transcription factors, such as CHOP, which is under both translational and transcriptional control (9). Phosphorylation of eIF2α thus leads to preferential translation of key regulatory molecules and directs diverse changes in the transcriptome of cells upon cellular stress.

One of the eIF2α kinases, PERK, lies at the intersection of the ISR and the unfolded protein response (UPR) that maintains homeostasis of protein folding rates in the ER (10). The UPR is activated by unfolded or misfolded proteins that accumulate in the ER lumen because of an imbalance between protein folding load and protein folding capacity, a condition known as “ER stress”. In mammals, the UPR is comprised of three signaling branches mediated by ER-localized transmembrane sensors, PERK, IRE1, and ATF6. These sensor proteins detect the accumulation of unfolded protein in the ER and transmit the information across the ER membrane, initiating unique signaling pathways that converge in the activation of an extensive transcriptional response, which ultimately results in ER expansion (11). The lumenal stress-sensing domains of PERK and IRE1 are homologous and likely activated in analogous ways by direct binding to unfolded peptides (12). This binding event leads to oligomerization and trans-autophosphorylation of their cytosolic kinase domains, and, for PERK, phosphorylation of its only known substrate, eIF2α. In this way, PERK activation results in a quick reduction in the load of newly synthesized proteins that are translocated into the ER-lumen (13).

Upon ER stress, both the transcription factor XBP1 s, produced as the consequence of a non-conventional mRNA splicing reaction initiated by IRE1, and the transcription factor ATF6, produced by proteolysis and release from the ER membrane, collaborate with ATF4 to induce the vast UPR transcriptional response. Transcriptional targets of the UPR include the ER protein folding machinery, the ER-associated degradation machinery, and many other components functioning in the secretory pathway (14). Although the UPR initially mitigates ER stress and as such confers cytoprotection, persistent and severe ER stress leads to activation of apoptosis that eliminates damaged cells (15,16).

Small-molecule therapeutics that inhibit the UPR and/or the Integrated Stress Response could be used in cancer as a single agent or in combination with other chemotherapeutics (17, 18, 19), for enhancement of long-term memory (24,25), in neurodegenerative and prion associated diseases (20), in white matter disease (VWM) (23) and in biotechnology applications that would benefit from increased protein translation.

It is an object of the instant invention to provide novel compounds that prevent the translation of ATF4 or are inhibitors of the ATF4 pathway.

It is also an object of the present invention to provide pharmaceutical compositions that comprise a pharmaceutically acceptable excipient and compounds of Formula (I).

It is also an object of the present invention to provide a method for treating neurodegenerative diseases, cancer, and other diseases/injuries associated with activated unfolded protein response pathways such as: Alzheimer's disease, spinal cord injury, traumatic brain injury, ischemic stroke, stroke, diabetes, Parkinson disease, Huntington's disease, Creutzfeldt-Jakob Disease, and related prion diseases, amyotrophic lateral sclerosis, progressive supranuclear palsy, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementias, atherosclerosis, ocular diseases, neurological disorders, pain, arrhythmias, in organ transplantation and in the transportation of organs for transplantation that comprises administering novel inhibitors of the ATF4 pathway.

### SUMMARY OF THE INVENTION

The invention is directed to substituted carbon-linked bicycloalkane derivatives. Specifically, the invention is directed to compounds according to Formula (X):

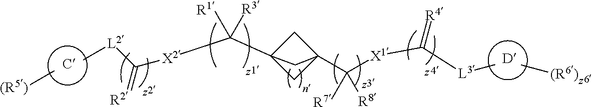

wherein C′, D′, L^{2′}, L^{3′}, R^{2′}, R^{3′}, R^{4′}, R^{5′}, R^{6′}, R^{7′}, R^{8′}, z²′, z³′, z^{4′}, z⁵′, z⁶′, X¹, and X² are as defined below; or a salt thereof including a pharmaceutically acceptable salt thereof.

The present invention also relates to the discovery that the compounds of Formula (X) are active as inhibitors of the ATF4 pathway.

The present invention also relates to the discovery that the compounds of Formula (X) prevent the translation of ATF4.

This invention also relates to a method of treating Alzheimer's disease, which comprises administering to a human in need thereof an effective amount of a compound of Formula (X) or a pharmaceutically acceptable salt thereof.

This invention also relates to a method of treating Parkinson's disease, which comprises administering to a human in need thereof an effective amount of a compound of Formula (X) or a pharmaceutically acceptable salt thereof.

This invention also relates to a method of treating amyotrophic lateral sclerosis, which comprises administering to a human in need thereof an effective amount of a compound of Formula (X) or a pharmaceutically acceptable salt thereof.

This invention also relates to a method of treating Huntington's disease, which comprises administering to a human in need thereof an effective amount of a compound of Formula (X) or a pharmaceutically acceptable salt thereof.

This invention also relates to a method of treating Creutzfeldt-Jakob Disease, which comprises administering to a human in need thereof an effective amount of a compound of Formula (X) or a pharmaceutically acceptable salt thereof.

This invention also relates to a method of treating progressive supranuclear palsy (PSP), which comprises administering to a human in need thereof an effective amount of a compound of Formula (X) or a pharmaceutically acceptable salt thereof.

This invention also relates to a method of treating dementia, which comprises administering to a human in need thereof an effective amount of a compound of Formula (X) or a pharmaceutically acceptable salt thereof.

This invention also relates to a method of treating spinal cord injury, which comprises administering to a human in need thereof an effective amount of a compound of Formula (X) or a pharmaceutically acceptable salt thereof.

This invention also relates to a method of treating traumatic brain injury, which comprises administering to a human in need thereof an effective amount of a compound of Formula (X) or a pharmaceutically acceptable salt thereof.

This invention also relates to a method of treating ischemic stroke, which comprises administering to a human in need thereof an effective amount of a compound of Formula (X) or a pharmaceutically acceptable salt thereof.

This invention also relates to a method of treating diabetes, which comprises administering to a human in need thereof an effective amount of a compound of Formula (X) or a pharmaceutically acceptable salt thereof.

This invention also relates to a method of treating a disease state selected from: myocardial infarction, cardiovascular disease, atherosclerosis, ocular diseases, and arrhythmias, which comprises administering to a human in need thereof an effective amount of a compound of Formula (X) or a pharmaceutically acceptable salt thereof.

This invention also relates to a method of treating an integrated stress response-associated disease in a patient in need of such treatment, which comprises administering a therapeutically effective amount of a compound of Formula (X) or a pharmaceutically acceptable salt thereof, to the patient.

This invention also relates to a method of treating a disease associated with phosphorylation of eIF2α in a patient in need of such treatment, which comprises administering a therapeutically effective amount of a compound of Formula (X), or a pharmaceutically acceptable salt thereof, to the patient.

This invention also relates to a method of treating a disease in a patient in need of such treatment, which comprises administering a therapeutically effective amount of a compound of Formula (X) or a pharmaceutically acceptable salt thereof, to the patient, wherein the disease is selected from the group consisting of cancer, a neurodegenerative disease, vanishing white matter disease, childhood ataxia with CNS hypomyelination, and an intellectual disability syndrome.

This invention also relates to a method of improving long-term memory in a patient, which comprises administering a therapeutically effective amount of a compound of Formula (X) or a pharmaceutically acceptable salt thereof, to the patient.

This invention also relates to a method of increasing protein expression of a cell or in vitro expression system, which comprises administering an effective amount of a compound of Formula (X) or a pharmaceutically acceptable salt thereof, to the cell or expression system.

This invention also relates to a method of treating an inflammatory disease in a patient in need of such treatment, which comprises administering a therapeutically effective amount of a compound of Formula (X), or a pharmaceutically acceptable salt thereof, to the patient.

This invention also relates to a method of using the compounds of Formula (X) in organ transplantation and in the transportation of organs for transplantation.

Also included in the present invention are methods of co-administering the presently invented compounds with further active ingredients.

Included in the present invention is a method for treating neurodegenerative diseases, cancer, and other diseases/injuries associated with activated unfolded protein response pathways, such as: Alzheimer's disease, spinal cord injury, traumatic brain injury, ischemic stroke, stroke, diabetes, Parkinson disease, Huntington's disease, Creutzfeldt-Jakob Disease, and related prion diseases, amyotrophic lateral sclerosis, progressive supranuclear palsy, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementias, atherosclerosis, ocular diseases, arrhythmias, in organ transplantation and in the transportation of organs for transplantation that comprises administering the compounds of Formula (X).

The invention also relates to a compound of Formula (X) or a pharmaceutically acceptable salt thereof for use in therapy.

The invention also relates to a compound of Formula (X) or a pharmaceutically acceptable salt thereof for use in the treatment of Alzheimer's disease.

The invention also relates to a compound of Formula (X) or a pharmaceutically acceptable salt thereof for use in the treatment of Parkinson's disease syndromes.

The invention also relates to a compound of Formula (X) or a pharmaceutically acceptable salt thereof for use in the treatment of amyotrophic lateral sclerosis.

The invention also relates to a compound of Formula (X) or a pharmaceutically acceptable salt thereof for use in the treatment of Huntington's disease.

The invention also relates to a compound of Formula (X) or a pharmaceutically acceptable salt thereof for use in the treatment of Creutzfeldt-Jakob Disease.

The invention also relates to a compound of Formula (X) or a pharmaceutically acceptable salt thereof for use in the treatment of progressive supranuclear palsy (PSP).

The invention also relates to a compound of Formula (X) or a pharmaceutically acceptable salt thereof for use in the treatment of dementia.

The invention also relates to a compound of Formula (X) or a pharmaceutically acceptable salt thereof for use in the treatment of spinal cord injury.

The invention also relates to a compound of Formula (X) or a pharmaceutically acceptable salt thereof for use in the treatment of traumatic brain injury.

The invention also relates to a compound of Formula (X) or a pharmaceutically acceptable salt thereof for use in the treatment of ischemic stroke.

The invention also relates to a compound of Formula (X) or a pharmaceutically acceptable salt thereof for use in the treatment of diabetes.

The invention also relates to a compound of Formula (X) or a pharmaceutically acceptable salt thereof for use in the treatment of a disease state selected from: myocardial infarction, cardiovascular disease, atherosclerosis, ocular diseases, and arrhythmias.

The invention also relates to the use of a compound of Formula (X) or a pharmaceutically acceptable salt thereof in the manufacture of a medicament for the treatment of an integrated stress response-associated disease.

The invention also relates to the use of a compound of Formula (X) or a pharmaceutically acceptable salt thereof in the manufacture of a medicament for the treatment of a disease associated with phosphorylation of eIF2α.

The invention also relates to the use of a compound of Formula (X) or a pharmaceutically acceptable salt thereof in the manufacture of a medicament for the treatment of a disease selected from the group consisting of: cancer, a neurodegenerative disease, vanishing white matter disease, childhood ataxia with CNS hypomyelination, and an intellectual disability syndrome.

The invention also relates to the use of a compound of Formula (X) or a pharmaceutically acceptable salt thereof in the manufacture of a medicament for improving long-term memory.

The invention also relates to the use of a compound of Formula (X) or a pharmaceutically acceptable salt thereof in the manufacture of a medicament for increasing protein expression of a cell or in vitro expression system.

The invention also relates to the use of a compound of Formula (X) or a pharmaceutically acceptable salt thereof in the manufacture of a medicament for the treatment of inflammatory disease.

The invention also relates to the use of a compound of Formula (X) or a pharmaceutically acceptable salt thereof in the manufacture of a medicament in organ transplantation and in the transportation of organs for transplantation.

The invention also relates to the use of a compound of Formula (X) or a pharmaceutically acceptable salt thereof in the manufacture of a medicament for the treatment of a disease state selected from: neurodegenerative diseases, cancer, and other diseases/injuries associated with activated unfolded protein response pathways such as: Alzheimer's disease, spinal cord injury, traumatic brain injury, ischemic stroke, stroke, diabetes, Parkinson disease, Huntington's disease, Creutzfeldt-Jakob Disease, and related prion diseases, amyotrophic lateral sclerosis, progressive supranuclear palsy, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementias, atherosclerosis, ocular diseases, neurological disorders, pain, arrhythmias, in organ transplantation and in the transportation of organs for transplantation.

Included in the present invention are pharmaceutical compositions that comprise a pharmaceutical excipient and a compound of Formula (X) or a pharmaceutically acceptable salt thereof.

The invention also relates to a pharmaceutical composition as defined above for use in therapy.

The invention also relates to a combination for use in therapy which comprises a therapeutically effective amount of (i) a compound of Formula (X) or a pharmaceutically acceptable salt thereof; and (ii) further active ingredients.

### DETAILED DESCRIPTION OF THE INVENTION

Included in the compounds of the invention and used in the methods of the invention are compounds of Formula (X):

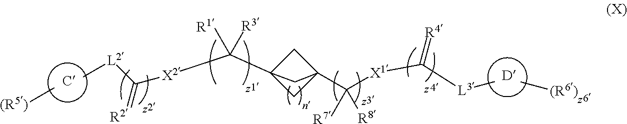

wherein:
L^{2′} is selected from: a bond, —NH—, —N(C₁₋₄alkyl)-, —N(substituted C₁₋₄alkyl)-, —O—, —S—, —S(O)—, —S(O)₂—, cycloalkyl, —O-cycloalkyl, cycloalkyl-O—, —NH-cycloalkyl, cycloalkyl-NH—, —CH₂-cycloalkyl, cycloalkyl-CH₂—, azetidinyl, —O-azetidinyl, azetidinyl-O—, —N-azetidinyl, azetidinyl-N—, substituted or unsubstituted C₁₋₆alkylene and substituted or unsubstituted C₁₋₆heteroalkylene,
   or,
   L^{2′} is taken together with R^{c′} to form:
   heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—,
   or,
   L^{2′} is taken together with an R^{5′} substituent adjacent to the point of attachment of L^{2′} to C′ to form a cycloalkyl ring fused to C′, a heterocycloalkyl ring fused to C′, or a heteroaryl ring fused to C′, wherein said ring fused to C′ is optionally subsitituted with from 1 to 3 substituents independently selected from: F, —CH₃, —CF₃, oxo, —OH and —OCH₃;
L^{3′} is selected from: a bond, —NH—, —N(C₁₋₄alkyl)-, —N(substituted C₁₋₄alkyl)-, —O—, —S—, —S(O)—, —S(O)₂—, cycloalkyl, —O-cycloalkyl, cycloalkyl-O—, —NH— cycloalkyl, cycloalkyl-NH—, —CH₂-cycloalkyl, cycloalkyl-CH₂—, azetidinyl, —O-azetidinyl, azetidinyl-O—, —N-azetidinyl, azetidinyl-N—, substituted or unsubstituted C₁₋₆alkylene and substituted or unsubstituted C₁₋₆heteroalkylene,
   or,
   L^{3′} is taken together with R^{b′} to form:
   heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—,
   or,
   L^{3′} is taken together with an R^{6′} substituent adjacent to the point of attachment of L^{3′} to D′ to form a cycloalkyl ring fused to D′, a heterocycloalkyl ring fused to D′, or heteroaryl ring fused to D′, wherein said ring fused to D′ is optionally subsitituted with from 1 to 3 substituents independently selected from: F, —CH₃, —CF₃, oxo, —OH and —OCH₃;
R^{1′} and R^{3′} are independently selected from: hydrogen, substituted or unsubstituted C₁₋₆alkyl, or R^{1′} and R^{3′} are taken together with the carbon to which they are attached to form a substituted or unsubstituted cycloalkyl, substituted or unsubstituted heterocycloalkyl, substituted or unsubstituted aryl, or substituted or unsubstituted heteroaryl;
R^{2′} and R^{4′} are independently NR^{a′}, O, or S;
   R^{a′} is selected from: hydrogen, C₁₋₆alkyl and C₁₋₆alkyl substituted 1 to 6 times by fluoro;
R^{5′} is selected from: fluoro, chloro, bromo, iodo, —C(O)OC₁₋₄alkyl, —OH, —NH₂, —C(O)NHC₁₋₄alkyl, —OCH₂Ph, —C(O)Ph, —CF₃, —CN, —S(O)CH₃, —C(O)OH, —CONH₂, —NO₂, —C(O)CH₃, —C≡CH, —CH₂C≡CH, —SCH₃, —SO₃H, —SO₂NH₂, —NHC(O)NH₂, —NHC(O)H, —NHOH, —OCF₃, —OCHF₂, —C(OH)R^{x}R^{y} (where R^{x} is selected from hydrogen, C₁₋₄alkyl, and cycloalkyl, and R^{y} is selected from C₁₋₄alkyl, and cycloalkyl), substituted or unsubstituted C₁₋₆alkyl, substituted or unsubstituted C₁₋₆heteroalkyl, substituted or unsubstituted cycloalkyl, substituted or unsubstituted heterocycloalkyl, substituted or unsubstituted aryl, or substituted or unsubstituted heteroaryl,
   or,
   two adjacent R^{5′} substituents can combine to form a cycloalkyl ring, a heterocycloalkyl ring, or a heteroaryl ring fused to C′,
      wherein each of said rings fused to C′ is optionally subsitituted with from 1 to 3 substituents independently selected from: F, —CH₃, —CF₃, oxo, —OH and —OCH₃,
   or,
   an R^{5′} substituent adjacent to the point of attachment of L^{2′} to C′ combines with L^{2′} to form a cycloalkyl ring fused to C′, a heterocycloalkyl ring fused to C′, or a heteroaryl ring fused to C′,
      wherein said ring fused to C′ is optionally subsitituted with from 1 to 3 substituents independently selected from: F, —CH₃, —CF₃, oxo, —OH and —OCH₃;
R^{6′} is selected from: fluoro, chloro, bromo, iodo, —C(O)OC₁₋₄alkyl, —OH, —NH₂, —C(O)NHC₁₋₄alkyl, —OC₁₋₄alkyl, —OCH₂Ph, —C(O)Ph, —CF₃, —CN, —S(O)CH₃, —C(O)OH, —CONH₂, —NO₂, —C(O)CH₃, —CH₂C≡CH, —SCH₃, —SO₃H, —SO₂NH₂, —NHC(O)NH₂, —NHC(O)H, —NHOH, —OCF₃, —OCHF₂, —C(OH)R^{x}R^{y} (where R^{x} is selected from hydrogen, C₁₋₄alkyl, and cycloalkyl, and R^{y} is selected from C₁₋₄alkyl, and cycloalkyl), substituted or unsubstituted C₁₋₆alkyl, substituted or unsubstituted C₁₋₆heteroalkyl, substituted or unsubstituted cycloalkyl, substituted or unsubstituted heterocycloalkyl, substituted or unsubstituted aryl, or substituted or unsubstituted heteroaryl,
   or,
   two adjacent R^{6′} substituents combine to form a cycloalkyl ring, a heterocycloalkyl ring, or heteroaryl ring fused to D′,
      wherein each of said rings fused to D′ is optionally subsitituted with from 1 to 3 substituents independently selected from: F, —CH₃, —CF₃, oxo, —OH and —OCH₃,
   or,
   an R^{6′} substituent adjacent to the point of attachment of L^{3′} to D′ combines with L^{3′} to form a cycloalkyl ring fused to D′, a heterocycloalkyl ring fused to D′, or a heteroaryl ring fused to D,
      wherein said ring fused to D′ is optionally subsitituted with from 1 to 3 substituents independently selected from: F, —CH₃, —CF₃, oxo, —OH and —OCH₃;
R^{7′} and R^{8′} are independently selected from: hydrogen, substituted or unsubstituted C₁₋₆alkyl, or R^{7′} and R^{8′} are taken together with the carbon to which they are attached to form a substituted or unsubstituted cycloalkyl, substituted or unsubstituted heterocycloalkyl, substituted or unsubstituted aryl, or substituted or unsubstituted heteroaryl;
C′ and D′ are independently phenyl or pyridyl;
X^{1′} is selected from: —O—, —NH—, and —NR^{b′}—;
   R^{b′} is selected from: C₁₋₆alkyl, substituted C₁₋₆alkyl, cycloalkyl, and heterocycloalkyl, or R^{b′} is taken together with L^{3′} to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
X^{2′} is selected from: —O—, —NH—, and —NR^{c′}—;
   R^{c′} is selected from: C₁₋₆alkyl, substituted C₁₋₆alkyl, cycloalkyl, and heterocycloalkyl, or R^{c′} is taken together with L^{2′} to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
n′ is 1 or 2;
z¹, z²′, z^{3′} and z^{4′} are independently 0 or 1; and
z⁵ and z⁶ are independently an integer from 0 to 5;
   provided at least one of z^{1′} and z³ is 1;
   or a salt thereof including a pharmaceutically acceptable salt thereof.

This invention also relates to pharmaceutically acceptable salts of the compounds of Formula (X).

For compounds of Formula (X), suitably n′ is 1.

For compounds of Formula (X), suitably n′ is 2.
For compounds of Formula (X), suitably L^{2′} is selected from: a bond, —CH₂—, —NH—, —NH—CH₂—CH₂—, —CH₂—CH₂—NH—, —O—, —CH₂—O—, —O—CH₂—, —O—CH₂—CH₂—, —CH₂—CH₂—O—, cyclopropyl, —O-cyclopropyl, cyclopropyl-O—, —CH₂-cyclopropyl, and cyclopropyl-CH₂—.
For compounds of Formula (X), suitably L^{3′} is selected from: a bond, —CH₂—, —NH—, —NH—CH₂—CH₂—, —CH₂—CH₂—NH—, —O—, —CH₂—O—, —O—CH₂—, —O—CH₂—CH₂—, —CH₂—CH₂—O—, cyclopropyl, —O-cyclopropyl, cyclopropyl-O—, —CH₂-cyclopropyl, and cyclopropyl-CH₂—,
   or,
   L^{3′} is taken together with an R^{6′} substituent adjacent to the point of attachment of L^{3′} to D′ to form a heterocycloalkyl ring fused to D′, wherein said ring fused to D is optionally subsitituted with 1 substituent selected from: F, —CH₃, —CF₃, oxo, —OH and —OCH₃.
For compounds of Formula (X), suitably L^{3′} is selected from: a bond, —CH₂—O—, —O—CH₂—, —O—, —CH₂—NH—, —NH—CH₂—, and —NH—,
   or,
   L^{3′} is taken together with an R^{6′} substituent adjacent to the point of attachment of L^{3′} to D′ to form: 1,4-oxazinyl, 1,4-oxazinyl subsitituted by methyl, tetrahydropyranyl or 1,4-dioxanyl.
For compounds of Formula (X), suitably R^{1′} and R^{3′} are independently selected from: hydrogen, C₁₋₄alkyl, C₁₋₄alkyl substituted from 1 to 3 times by fluoro, or R^{1′} and R^{3′} are taken together with the carbon to which they are attached to form cyclopropyl.
For compounds of Formula (X), suitably L^{2′} is selected from: a bond, —NH—, —CH₂—O— or —O—CH₂—.
For compounds of Formula (X), suitably L^{3′} is selected from: a bond, —NH—, —CH₂—O— or —O—CH₂—.
For compounds of Formula (X), suitably L^{3′} is taken together with an R^{6′} substituent adjacent to the point of attachment of L^{3′} to D′ to form a heterocycloalkyl ring fused to D′, wherein said ring fused to D′ is selected from: 1,4-oxazinyl, 1,4-oxazinyl subsitituted by methyl, tetrahydropyranyl or 1,4-dioxanyl.
For compounds of Formula (X), suitably z^{1′} is 1 and R^{1′} and R^{3′} are independently selected from: hydrogen, C₁₋₆alkyl, and C₁₋₆alkyl substituted with from 1 to 3 substituents independently selected from: —OH, —NH₂, —NHC₁₋₄alkyl, —OC₁₋₄alkyl and —OC₁₋₄alkyl substituted with —OC₁₋₃alkyl.
For compounds of Formula (X), suitably z¹ is 1 and z³ is 0.
For compounds of Formula (X), suitably R^{2′} and R^{4′} are independently 0 or S.
For compounds of Formula (X), suitably R^{2′} and R^{4′} are 0.
For compounds of Formula (X), suitably each R^{5′} is fluoro or chloro.
For compounds of Formula (X), suitably R^{5′} is selected from: fluoro, chloro, bromo, —CF₃ and —CH₃.
   For compounds of Formula (X), suitably R^{6′} is selected from: fluoro, chloro, bromo, —CF₃ and —CH₃.
   For compounds of Formula (X), suitably R^{5′} is selected from: fluoro, chloro, bromo, —CH₃, —CF₂H, —OCF₃ and —CF₃.
   For compounds of Formula (X), suitably R^{6′} is selected from: fluoro, chloro, bromo, —CH₃, —OCF₃, —CF₂H and —CF₃,
      or,
      two adjacent R^{6′} substituents can combine to form a dioxole ring fused to D, wherein said ring fused to D is optionally subsitituted 2 times by F,
      or,
   an R^{6′} substituent, adjacent to the point of attachment of L^{3′} to D′, is taken together with L^{3′} to form 1,4-oxazinyl, 1,4-oxazinyl subsitituted by tetrahydropyranyl or 1,4-dioxanyl.
   For compounds of Formula (X), suitably R^{6′} is selected from: fluoro, chloro, bromo, —CH₃, —OCF₃, —CF₂H and —CF₃,
      or,
      an R^{6′} substituent, adjacent to the point of attachment of L^{3′} to D′, is taken together with L^{3′} to form 1,4-oxazinyl, 1,4-oxazinyl subsitituted by methyl, tetrahydropyranyl or 1,4-dioxanyl.
For compounds of Formula (X), suitably R^{7′} and R^{8′} are independently selected from: hydrogen, C₁₋₄alkyl, C₁₋₄alkyl substituted from 1 to 3 times by fluoro, or R^{1′} and R^{3′} are taken together with the carbon to which they are attached to form cyclopropyl.
For compounds of Formula (X), suitably C′ and D′ are phenyl.
For compounds of Formula (X), suitably C′ is phenyl.
For compounds of Formula (X), suitably D′ is phenyl or pyridyl.
For compounds of Formula (X), suitably C and D are each independently selected from: phenyl and pyridyl.
   For compounds of Formula (X), suitably X^{1′} is selected from: —O—, —NH— and —N(CH₃)—.
   For compounds of Formula (X), suitably X^{1′} is —NH—.
   For compounds of Formula (X), suitably X^{2′} is selected from: —O—, —NH— and —NR^{c}—, where R^{c} is —CH₃, or R^{c} is taken together with L^{2′} to for: oxopyrrolidinyl-O—.
   For compounds of Formula (X), suitably X^{2′} is selected from: —O—, —NH— and —N(CH₃)—.

For compounds of Formula (X), suitably X^{1′} and X^{2′} is independently selected from: —O— and —NH—.
For compounds of Formula (X), suitably X^{2′} is —O—.
For compounds of Formula (X), suitably z^{2′} and z^{1′} are 1.
For compounds of Formula (X), suitably z^{3′} is 0 and z^{1′} is 1.
For compounds of Formula (X), suitably z^{2′} and z^{4′} are both 1.
For compounds of Formula (X), suitably z^{5′} and z^{6′} are independently an integer from 1 to 3.
For compounds of Formula (X), suitably z^{5′} and z^{6′} are independently 1 or 2.

Included in the compounds of the invention and used in the methods of the invention are compounds of Formula (XI):

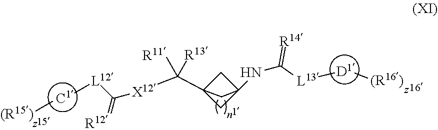

wherein:
L^{12′} is selected from: a bond, —NH—, —N(C₁₋₄alkyl)-, —N(substituted C₁₋₄alkyl)-, —O—, —S—, —S(O)—, —S(O)₂—, cycloalkyl, —O-cycloalkyl, cycloalkyl-O—, —NH-cycloalkyl, cycloalkyl-NH—, —CH₂-cycloalkyl, cycloalkyl-CH₂—, azetidinyl, —O-azetidinyl, azetidinyl-O—, —N-azetidinyl, azetidinyl-N—, substituted or unsubstituted C₁₋₆alkylene and substituted or unsubstituted C₁₋₆heteroalkylene,
   or,
   L^{12′} is taken together with R^{c1′} to form:
   heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—,
   or,
   L^{12′} is taken together with an R^{15′} substituent adjacent to the point of attachment of L^{12′} to C^{1′} to form a cycloalkyl ring fused to C^{1′}, a heterocycloalkyl ring fused to C^{1′}, or a heteroaryl ring fused to C^{1′}, wherein said ring fused to C^{1′} is optionally subsitituted with from 1 to 3 substituents independently selected from: F, —CH₃, —CF₃, oxo, —OH and —OCH₃;
L^{13′} is selected from: a bond, —NH—, —N(C₁₋₄alkyl)-, —N(substituted C₁₋₄alkyl)-, —O—, —S—, —S(O)—, —S(O)₂—, cycloalkyl, —O-cycloalkyl, cycloalkyl-O—, —NH-cycloalkyl, cycloalkyl-NH—, —CH₂-cycloalkyl, cycloalkyl-CH₂—, azetidinyl, —O-azetidinyl, azetidinyl-O—, —N-azetidinyl, azetidinyl-N—, substituted or unsubstituted C₁₋₆alkylene and substituted or unsubstituted C₁₋₆heteroalkylene,
   or,
   L^{13′} is taken together with an R^{16′} substituent adjacent to the point of attachment of L^{13′} to D^{1′} to form a cycloalkyl ring fused to D^{1′}, a heterocycloalkyl ring fused to D^{1′}, or a heteroaryl ring fused to D^{1′}, wherein said ring fused to D^{1′} is optionally subsitituted with from 1 to 3 substituents independently selected from: F, —CH₃, —CF₃, oxo, —OH and —OCH₃;
R^{11′} and R^{13′} are independently selected from: hydrogen, C₁₋₆alkyl, C₁₋₆alkyl substituted from 1 to 3 times by fluoro, or R^{11′} and R^{13′} are taken together with the carbon to which they are attached to form a cycloalkyl, or heterocycloalkyl;
R^{12′} and R^{14′} are independently O, or S;
R^{15′} is selected from: fluoro, chloro, bromo, iodo, C₁₋₄alkyl, —C(O)OC₁₋₄alkyl, —OH, —NH₂, —C(O)NHC₁₋₄alkyl, —OC₁₋₄alkyl, —OCH₂Ph, —C(O)Ph, —CF₃, —CN, —S(O)CH₃, —C(O)OH, —CONH₂, —NO₂, —C(O)CH₃, —CH₂C≡CH, —SCH₃, —SO₃H, —SO₂NH₂, —NHC(O)NH₂, —NHC(O)H, —NHOH, —OCF₃, —OCHF₂,
   or,
   an R^{15′} substituent adjacent to the point of attachment of L^{12′} to C^{1′} is taken together with L^{12′} to form a heterocycloalkyl ring fused to C^{1′}, wherein said ring fused to C^{1′} is optionally subsitituted with from 1 to 3 substituents independently selected from: F, —CH₃, —CF₃, oxo, —OH and —OCH₃;
R^{16′} is selected from: fluoro, chloro, bromo, iodo, C₁₋₄alkyl, —C(O)OC₁₋₄alkyl, —OH, —NH₂, —C(O)NHC₁₋₄alkyl, —OC₁₋₄alkyl, —OCH₂Ph, —C(O)Ph, —CF₃, —CN, —S(O)CH₃, —C(O)OH, —CONH₂, —NO₂, —C(O)CH₃, —CH₂C≡CH, —SCH₃, —SO₃H, —SO₂NH₂, —NHC(O)NH₂, —NHC(O)H, —NHOH, —OCF₃, —OCHF₂,
   or,
   two adjacent R^{16′} substituents can combine to form a heterocycloalkyl ring fused to D^{1′}, wherein said ring fused to Di is optionally subsitituted from 1 to 3 times by F,
   or,
   an R^{16′} substituent adjacent to the point of attachment of L^{13′} to D^{1′} is taken together with L^{13′} to form a heterocycloalkyl ring fused to D^{1′}, wherein said ring fused to D^{1′}, is optionally subsitituted with from 1 to 3 substituents independently selected from: F, —CH₃, —CF₃, oxo, —OH and —OCH₃;
C^{1′} and D^{1′} are independently phenyl or pyridyl;
X^{12′} is selected from: —O—, —NH—, and —NR^{c1′}—;
   R^{c1′} is selected from: C₁₋₆alkyl, C₁₋₄alkyl substituted from 1 to 3 times by fluoro, and cycloalkyl, or R^{c1} is taken together with L^{12′} to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
n^{1′} is 1 or 2; and
z^{15′} and z^{16′} are independently an integer from 0 to 4;
   or a salt thereof including a pharmaceutically acceptable salt thereof.

This invention also relates to pharmaceutically acceptable salts of the compounds of Formula (XI).

For compounds of Formula (XI), suitably nr is 1.

For compounds of Formula (XI), suitably nr is 2.
For compounds of Formula (XI), suitably L^{12′} is selected from: a bond, —CH₂—, —NH—, —CH₂—NH—, —NH—CH₂—, —NH—CH₂—CH₂—, —CH₂—CH₂—NH—, —O—, —CH₂—O—, —O—CH₂—, —O—CH₂—CH₂—, —CH₂—CH₂—O—, cyclopropyl, —O-cyclopropyl, cyclopropyl-O—, —CH₂-cyclopropyl, and cyclopropyl-CH₂—.
For compounds of Formula (XI), suitably L^{13′} is selected from: a bond, —CH₂—, —NH—, —CH₂—NH—, —NH—CH₂—, —NH—CH₂—CH₂—, —CH₂—CH₂—NH—, —O—, —CH₂—O—, —O—CH₂—, —O—CH₂—CH₂—, —CH₂—CH₂—O—, cyclopropyl, —O-cyclopropyl, cyclopropyl-O—, —CH₂-cyclopropyl, and cyclopropyl-CH₂—,
   or,
   L^{13′} is taken together with an R^{16′} substituent adjacent to the point of attachment of L^{13′} to D^{1′} to form a heterocycloalkyl ring fused to D^{1′}, wherein said ring fused to D^{1′} is optionally subsitituted with 1 substituent selected from: F, —CH₃, —CF₃, oxo, —OH and —OCH₃.
For compounds of Formula (XI), suitably L^{13′} is selected from: a bond, —CH₂—O—, —O—CH₂—, —O—, —CH₂—NH—, —NH—CH₂—, and —NH—,
   or,
   L^{13′} is taken together with an R^{16′} substituent adjacent to the point of attachment of L^{13′} to D^{1′} to form: 1,4-oxazinyl, 1,4-oxazinyl subsitituted by methyl, tetrahydropyranyl or 1,4-dioxanyl.
   For compounds of Formula (XI), suitably R^{11′} and R^{13′} are independently selected from: hydrogen, C₁₋₄alkyl, C₁₋₄alkyl substituted from 1 to 3 times by fluoro, or R^{11′} and R^{13′} are taken together with the carbon to which they are attached to form cyclo propyl.
For compounds of Formula (XI), suitably L^{12′} is selected from: a bond, —NH—, —CH₂—O— or —O—CH₂—.
For compounds of Formula (XI), suitably L^{13′} is selected from: a bond, —NH—, —CH₂—O— or —O—CH₂—.
For compounds of Formula (XI), suitably L^{13′} is taken together with an R^{16′} substituent adjacent to the point of attachment of L^{13′} to D^{1′} to form a heterocycloalkyl ring fused to D^{1′}, wherein said ring fused to D^{1′} is selected from: 1,4-oxazinyl, 1,4-oxazinyl subsitituted by methyl, tetrahydropyranyl or 1,4-dioxanyl.
For compounds of Formula (XI), suitably R^{11′} and R^{13′} are independently selected from: hydrogen, C₁₋₆alkyl, and C₁₋₆alkyl substituted with from 1 to 3 substituents independently selected from: —OH, —NH₂, —NHC₁₋₄alkyl, —OC₁₋₄alkyl and —OC₁₋₄alkyl substituted with —OC₁₋₃ alkyl.
For compounds of Formula (XI), suitably R^{12′} and R^{14′} are independently 0 or S.
For compounds of Formula (XI), suitably R^{12′} and R^{14′} are 0.
For compounds of Formula (XI), suitably each R^{15′} is fluoro or chloro.
For compounds of Formula (XI), suitably R^{15′} is selected from: fluoro, chloro, bromo, —CF₃ and —CH₃.
   For compounds of Formula (XI), suitably R^{16′} is selected from: fluoro, chloro, bromo, —CF₃ and —CH₃.
   For compounds of Formula (XI), suitably R^{15′} is selected from: fluoro, chloro, bromo, —CH₃, —CF₂H, —OCF₃ and —CF₃.
   For compounds of Formula (XI), suitably R^{16′} is selected from: fluoro, chloro, bromo, —CH₃, —OCF₃, —CF₂H and —CF₃,
      or,
      two adjacent R^{16′} substituents can combine to form a dioxole ring fused to D^{1′}, wherein said ring fused to D^{1′} is optionally subsitituted 2 times by F,
      or,
      an R^{16′} substituent, adjacent to the point of attachment of L^{13′} to D^{1′}, is taken together with L^{13′} to form 1,4-oxazinyl, 1,4-oxazinyl subsitituted by methyl, tetrahydropyranyl or 1,4-dioxanyl.
   For compounds of Formula (XI), suitably R^{16′} is selected from: fluoro, chloro, bromo, —CH₃, —OCF₃, —CF₂H and —CF₃,
      or,
      an R^{16′} substituent, adjacent to the point of attachment of L^{13′} to D^{1′}, is taken together with L^{13′} to form 1,4-oxazinyl, 1,4-oxazinyl subsitituted by tetrahydropyranyl or 1,4-dioxanyl.
For compounds of Formula (XI), suitably C^{1′} and D^{1′} are phenyl.
For compounds of Formula (XI), suitably C^{1′} is phenyl.
For compounds of Formula (XI), suitably D^{1′} is phenyl or pyridyl.
For compounds of Formula (XI), suitably X^{12′} is selected from: —O—, —NH— and —NR^{c′}—, where R^{c′} is —CH₃, or R^{c′} is taken together with L^{12′} to form ioxopyrrolid inyl-O—.
   For compounds of Formula (XI), suitably X^{12′} is selected from: —O—, —NH— and —N(CH₃)—.
   For compounds of Formula (XI), suitably X^{12′} is —O—.
   For compounds of Formula (XI), suitably X^{12′} is selected from: —O— and —NH—.
   For compounds of Formula (XI), suitably z^{15′} and z^{16′} are independently an integer from 1 to 3.
   For compounds of Formula (XI), suitably z^{15′} and z^{16′} are independently 1 or 2.

Included in the compounds of the invention and used in the methods of the invention are compounds of Formula (XII):

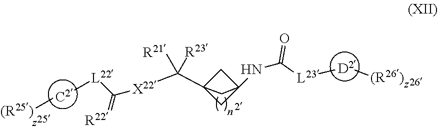

wherein:
L^{22′} is selected from: a bond, —CH₂—, —NH—, —CH₂—NH—, —NH—CH₂—, —NH—CH₂—CH₂—, —CH₂—CH₂—NH—, —O—, —CH₂—O—, —O—CH₂—, —O—CH₂—CH₂—, —CH₂—CH₂—O—, cyclopropyl, —O-cyclopropyl, cyclopropyl-O—, —CH₂-cyclopropyl, and cyclopropyl-CH₂—;
L^{23′} is selected from: a bond, —CH₂—, —NH—, —CH₂—NH—, —NH—CH₂—, —NH—CH₂—CH₂—, —CH₂—CH₂—NH—, —O—, —CH₂—O—, —O—CH₂—, —O—CH₂—CH₂—, —CH₂—CH₂—O—, cyclopropyl, —O-cyclopropyl, cyclopropyl-O—, —CH₂-cyclopropyl, and cyclopropyl-CH₂—,
   or,
   L^{23′} is taken together with an R^{26′} substituent adjacent to the point of attachment of L^{23′} to D^{2′} to form a heterocycloalkyl ring fused to D^{2′}′, wherein said ring fused to D^{2′} is optionally subsitituted with 1 substituent selected from: F, —CH₃, —CF₃, oxo, —OH and —OCH₃;
R^{21′} and R^{23′} are independently selected from: hydrogen, C₁₋₄alkyl, C₁₋₄alkyl substituted from 1 to 3 times by fluoro, or R^{21′} and R^{2′3} are taken together with the carbon to which they are attached to form cyclopropyl;
R^{22′} is 0 or S;
R^{25′} is selected from: fluoro, chloro, bromo, C₁₋₄alkyl, —OH, —NH₂, —CF₃, —CHF₂, —CFH₂, —CN, —NO₂, —OCF₃, and —OCHF₂;
R^{26′} is selected from: fluoro, chloro, bromo, C₁₋₄alkyl, —OH, —NH₂, —CF₃, —CHF₂, —CFH₂, —CN, —NO₂, —OCF₃, and —OCHF₂,
   or,
   two adjacent R^{26′} substituents can combine to form a dioxole ring fused to D^{2′}, wherein said ring fused to D^{2′} is optionally subsitituted 1 or 2 times by F,
   or,
   an R^{26′} substituent, adjacent to the point of attachment of L^{23′} to D^{2′}′, is taken together with L^{23′} to form a heterocycloalkyl ring fused to D^{2′}, wherein said ring fused to D^{2′} is optionally subsitituted with 1 substituent selected from: F, —CH₃, —CF₃, oxo, —OH and —OCH₃;
C^{2′} and D^{2′} are each independently phenyl or pyridyl;
X^{22′} is selected from: —O—, —NH—, and —NR^{c2′}—, where R^{c2′} is selected from: C₁₋₂alkyl and C₁₋₂alkyl substituted from 1 to 3 times by fluoro;
n² is 1 or 2; and
z²⁵ and z²⁶ are independently an integer from 0 to 3;
   or a salt thereof including a pharmaceutically acceptable salt thereof.

This invention also relates to pharmaceutically acceptable salts of the compounds of Formula (XII).
For compounds of Formula (XII), suitably n^{2′} is 1.
For compounds of Formula (XII), suitably n^{2′} is 2.
For compounds of Formula (XII), suitably L^{23′} is selected from: a bond, —CH₂—O—, —O—CH₂—, —O—, —CH₂—NH—, —NH—CH₂—, and —NH—,
   or,
   L^{23′} is taken together with an R^{26′} substituent adjacent to the point of attachment of L^{23′} to D^{2′} to form: 1,4-oxazinyl, 1,4-oxazinyl subsitituted by methyl, tetrahydropyranyl or 1,4-dioxanyl.
For compounds of Formula (XII), suitably L^{22′} is selected from: a bond, —NH—, —CH₂—O— or —O—CH₂—.
For compounds of Formula (XII), suitably L^{23′} is selected from: a bond, —NH—, —CH₂—O— or —O—CH₂—.
For compounds of Formula (XII), suitably L^{23′} is taken together with an R^{26′} substituent adjacent to the point of attachment of L^{23′} to D^{2′} to form a heterocycloalkyl ring fused to D^{2′}′, wherein said ring fused to D^{2′} is selected from: 1,4-oxazinyl, 1,4-oxazinyl subsitituted by methyl, tetrahydropyranyl or 1,4-dioxanyl.
For compounds of Formula (XII), suitably R^{21′} and R^{23′} are independently selected from: hydrogen, C₁₋₆alkyl, and C₁₋₆alkyl substituted with from 1 to 3 substituents independently selected from: —OH, —NH₂, —NHC₁₋₄alkyl, —OC₁₋₄alkyl and —OC₁₋₄alkyl substituted with —OC₁₋₃ alkyl.
For compounds of Formula (XII), suitably R^{22′} is 0.
For compounds of Formula (XII), suitably each R^{25′} is fluoro or chloro.
For compounds of Formula (XII), suitably R^{25′} is selected from: fluoro, chloro, bromo, —CF₃ and —CH₃.
   For compounds of Formula (XII), suitably R^{26′} is selected from: fluoro, chloro, bromo, —CF₃ and —CH₃.
   For compounds of Formula (XII), suitably R^{25′} is selected from: fluoro, chloro, bromo, —CH₃, —CF₂H, —OCF₃ and —CF₃.
   For compounds of Formula (XII), suitably R^{26′} is selected from: fluoro, chloro, bromo, —CH₃, —OCF₃, —CF₂H and —CF₃,
      or,
      two adjacent R^{26′} substituents can combine to form a dioxole ring fused to D^{2′}′, wherein said ring fused to D^{1′} is optionally subsitituted 2 times by F,
      or,
      an R^{26′} substituent, adjacent to the point of attachment of L^{23′} to D^{2′}′, is taken together with L^{23′} to form 1,4-oxazinyl, 1,4-oxazinyl subsitituted by methyl, tetrahydropyranyl or 1,4-dioxanyl.
   For compounds of Formula (XII), suitably R^{26′} is selected from: fluoro, chloro, bromo, —CH₃, —OCF₃, —CF₂H and —CF₃,
      or,
      an R^{26′} substituent, adjacent to the point of attachment of L^{23′} to D^{2′}′, is taken together with L^{23′} to form 1,4-oxazinyl, 1,4-oxazinyl subsitituted by methyl, tetrahydropyranyl or 1,4-dioxanyl.
For compounds of Formula (XII), suitably C^{2′} and D^{2′} are phenyl.
For compounds of Formula (XII), suitably C^{2′} is phenyl.
For compounds of Formula (XII), suitably D^{2′} is phenyl or pyridyl.
For compounds of Formula (XI), suitably X^{12′} is selected from: —O—, —NH— and —N(CH₃)—.
   For compounds of Formula (XII), suitably X^{22′} is —O—.
   For compounds of Formula (XII), suitably X^{22′} is selected from: —O— and —NH—.
   For compounds of Formula (XII), suitably z^{25′} and z^{26′} are independently an integer from 1 to 3.
   For compounds of Formula (XII), suitably z^{25′} and z^{26′} are independently 1 or 2.

Included in the compounds of the invention and used in the methods of the invention are compounds of Formula (I):

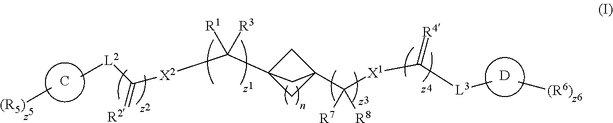

wherein:
L² is selected from: a bond, —NH—, —N(C₁₋₄alkyl)-, —N(substituted C₁₋₄alkyl)-, —O—, —S—, —S(O)—, —S(O)₂—, cycloalkyl, —O-cycloalkyl, cycloalkyl-O—, —NH-cycloalkyl, cycloalkyl-NH—, —CH₂-cycloalkyl, cycloalkyl-CH₂—, azetidinyl, —O-azetidinyl, azetidinyl-O—, —N-azetidinyl, azetidinyl-N—, substituted or unsubstituted C₁₋₆alkylene and substituted or unsubstituted C₁₋₆heteroalkylene,
   or,
   L² is taken together with R^{c} to form:
   heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—,
   or,
   L² is taken together with an R⁵ substituent adjacent to the point of attachment of L² to C to form a cycloalkyl ring, a heterocycloalkyl ring, or heteroaryl ring fused to C;
L³ is selected from: a bond, —NH—, —N(C₁₋₄alkyl)-, —N(substituted C₁₋₄alkyl)-, —O—, —S—, —S(O)—, —S(O)₂—, cycloalkyl, —O-cycloalkyl, cycloalkyl-O—, —NH— cycloalkyl, cycloalkyl-NH—, —CH₂-cycloalkyl, cycloalkyl-CH₂—, azetidinyl, —O-azetidinyl, azetidinyl-O—, —N-azetidinyl, azetidinyl-N—, substituted or unsubstituted C₁₋₆alkylene and substituted or unsubstituted C₁₋₆heteroalkylene,
   or,
   L³ is taken together with R^{b} to form:
   heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—,
   or,
   L³ is taken together with an R⁶ substituent adjacent to the point of attachment of L³ to D to form a cycloalkyl ring, a heterocycloalkyl ring, or heteroaryl ring fused to D;
R¹ and R³ are independently selected from: hydrogen, substituted or unsubstituted C₁₋₆alkyl, or R¹ and R³ are taken together with the carbon to which they are attached to form a substituted or unsubstituted cycloalkyl, substituted or unsubstituted heterocycloalkyl, substituted or unsubstituted aryl, or substituted or unsubstituted heteroaryl;
R² and R⁴ are independently NR^{a}, O, or S;
   R^{a} is selected from: hydrogen, C₁₋₆alkyl and C₁₋₆alkyl substituted 1 to 6 times by fluoro;
R⁵ is selected from: fluoro, chloro, bromo, iodo, —C(O)OC₁₋₄alkyl, —OH, —NH₂, —C(O)NHC₁₋₄alkyl, —OC₁₋₄alkyl, —OCH₂Ph, —C(O)Ph, —CF₃, —CN, —S(O)CH₃, —C(O)OH, —CONH₂, —NO₂, —C(O)CH₃, —C≡CH, —CH₂C≡CH, —SCH₃, —SO₃H, —SO₂NH₂, —NHC(O)NH₂, —NHC(O)H, —NHOH, —OCF₃, —OCHF₂, —C(OH)R^{x}R^{y} (where R^{x} is selected from hydrogen, C₁₋₄alkyl, and cycloalkyl, and R^{y} is selected from C₁₋₄alkyl, and cycloalkyl), substituted or unsubstituted C₁₋₆alkyl, substituted or unsubstituted C₁₋₆heteroalkyl, substituted or unsubstituted cycloalkyl, substituted or unsubstituted heterocycloalkyl, substituted or unsubstituted aryl, or substituted or unsubstituted heteroaryl,
   or,
   two adjacent R⁵ substituents can combine to form a cycloalkyl ring, a heterocycloalkyl ring, or a heteroaryl ring fused to C,
   or,
   an R⁵ substituent adjacent to the point of attachment of L² to C combines with L² to form a cycloalkyl ring, a heterocycloalkyl ring, or a heteroaryl ring fused to C;
R⁶ is selected from: fluoro, chloro, bromo, iodo, —C(O)OC₁₋₄alkyl, —OH, —NH₂, —C(O)NHC₁₋₄alkyl, —OC₁₋₄alkyl, —OCH₂Ph, —C(O)Ph, —CF₃, —CN, —S(O)CH₃, —C(O)OH, —CONH₂, —NO₂, —C(O)CH₃, —CH₂C≡CH, —SCH₃, —SO₃H, —SO₂NH₂, —NHC(O)NH₂, —NHC(O)H, —NHOH, —OCF₃, —OCHF₂, —C(OH)R^{x}R^{y} (where R^{x} is selected from hydrogen, C₁₋₄alkyl, and cycloalkyl, and R^{y} is selected from C₁₋₄alkyl, and cycloalkyl), substituted or unsubstituted C₁₋₆alkyl, substituted or unsubstituted C₁₋₆heteroalkyl, substituted or unsubstituted cycloalkyl, substituted or unsubstituted heterocycloalkyl, substituted or unsubstituted aryl, or substituted or unsubstituted heteroaryl,
   or,
   two adjacent R⁶ substituents combine to form a cycloalkyl ring, a heterocycloalkyl ring, or heteroaryl ring fused to D,
   or,
   an R⁶ substituent adjacent to the point of attachment of L³ to D combines with L³ to form a cycloalkyl ring, a heterocycloalkyl ring, or a heteroaryl ring fused to D;
R⁷ and R⁸ are independently selected from: hydrogen, substituted or unsubstituted C₁₋₆alkyl, or R⁷ and R⁸ are taken together with the carbon to which they are attached to form a substituted or unsubstituted cycloalkyl, substituted or unsubstituted heterocycloalkyl, substituted or unsubstituted aryl, or substituted or unsubstituted heteroaryl;
C and D are independently phenyl or pyridyl;
X¹ is selected from: —O—, —NH—, and —NR^{b}—;
   R^{b} is selected from: C₁₋₆alkyl, substituted C₁₋₆alkyl, cycloalkyl, and heterocycloalkyl, or R^{b} is taken together with L³ to form:
heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
X² is selected from: —O—, —NH—, and —NR^{c}—;
   R^{c} is selected from: C₁₋₆alkyl, substituted C₁₋₆alkyl, cycloalkyl, and heterocycloalkyl, or R^{c} is taken together with L² to form:
heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
n is 1 or 2;
z¹, z², z³ and z⁴ are independently 0 or 1; and
z⁵ and z⁶ are independently an integer from 0 to 5;
   provided at least one of z¹ and z³ is 1;
   or a salt thereof including a pharmaceutically acceptable salt thereof.

This invention also relates to pharmaceutically acceptable salts of the compounds of Formula (I).

Included in the compounds of the invention and used in the methods of the invention are compounds of Formula (II):

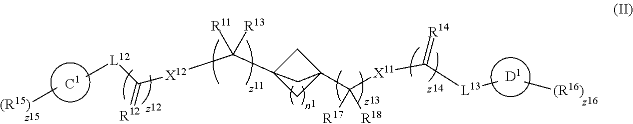

wherein:
L¹² is selected from: a bond, —NH—, —N(C₁₋₄alkyl)-, —N(substituted C₁₋₄alkyl)-, —O—, —S—, —S(O)—, —S(O)₂—, cycloalkyl, —O-cycloalkyl, cycloalkyl-O—, —NH-cycloalkyl, cycloalkyl-NH—, —CH₂-cycloalkyl, cycloalkyl-CH₂—, azetidinyl, —O-azetidinyl, azetidinyl-O—, —N-azetidinyl, azetidinyl-N—, substituted or unsubstituted C₁₋₆alkylene and substituted or unsubstituted C₁₋₆heteroalkylene,
   or,
   L¹² is taken together with R^{c1} to form:
   heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
L¹³ is selected from: a bond, —NH—, —N(C₁₋₄alkyl)-, —N(substituted C₁₋₄alkyl)-, —O—, —S—, —S(O)—, —S(O)₂—, cycloalkyl, —O-cycloalkyl, cycloalkyl-O—, —NH-cycloalkyl, cycloalkyl-NH—, —CH₂-cycloalkyl, cycloalkyl-CH₂—, azetidinyl, —O-azetidinyl, azetidinyl-O—, —N-azetidinyl, azetidinyl-N—, substituted or unsubstituted C₁₋₆alkylene and substituted or unsubstituted C₁₋₆heteroalkylene,
   or,
   L¹³ is taken together with R^{b1} to form:
   heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
R¹¹ and R¹³ are independently selected from: hydrogen, C₁₋₆alkyl, C₁₋₆alkyl substituted from 1 to 3 times by fluoro, or R¹¹ and R¹³ are taken together with the carbon to which they are attached to form a cycloalkyl, or heterocycloalkyl;
R¹² and R¹⁴ are independently O, or S;
R¹⁵ is selected from: fluoro, chloro, bromo, iodo, C₁₋₄alkyl, —C(O)OC₁₋₄alkyl, —OH, —NH₂, —C(O)NHC₁₋₄alkyl, —OC₁₋₄alkyl, —OCH₂Ph, —C(O)Ph, —CF₃, —CN, —S(O)CH₃, —C(O)OH, —CONH₂, —NO₂, —C(O)CH₃, —CH₂C≡CH, —SCH₃, —SO₃H, —SO₂NH₂, —NHC(O)NH₂, —NHC(O)H, —NHOH, —OCF₃, —OCHF₂, —C(OH)R^{x1}R^{y1} (where R^{x1} is selected from hydrogen, C₁₋₄alkyl, and cycloalkyl, and R^{y1} is selected from C₁₋₄alkyl, and cycloalkyl);
R¹⁶ is selected from: fluoro, chloro, bromo, iodo, C₁₋₄alkyl, —C(O)OC₁₋₄alkyl, —OH, —NH₂, —C(O)NHC₁₋₄alkyl, —OC₁₋₄alkyl, —OCH₂Ph, —C(O)Ph, —CF₃, —CN, —S(O)CH₃, —C(O)OH, —CONH₂, —NO₂, —C(O)CH₃, —CH₂Ca′CH, —SCH₃, —SO₃H, —SO₂NH₂, —NHC(O)NH₂, —NHC(O)H, —NHOH, —OCF₃, —OCHF₂, —C(OH)R^{x1}R^{y1} (where R^{x1} is selected from hydrogen, C₁₋₄alkyl, and cycloalkyl, and R^{y1} is selected from C₁₋₄alkyl, and cycloalkyl);
R¹⁷ and R¹⁸ are independently selected from: hydrogen, C₁₋₆alkyl, C₁₋₆alkyl substituted from 1 to 3 times by fluoro, or R¹⁷ and R¹⁸ are taken together with the carbon to which they are attached to form a cycloalkyl, or heterocycloalkyl;
C1 and D1 are independently phenyl or pyridyl;
X¹¹ is selected from: —O—, —NH—, and —NR^{b1}—;
   R^{b1} is selected from: C₁₋₆alkyl, C₁₋₄alkyl substituted from 1 to 3 times by fluoro, and cycloalkyl, or R^{b1} is taken together with L¹³ to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
X¹² is selected from: —O—, —NH—, and —NR^{c1}—;
   R^{c1} is selected from: C₁₋₆alkyl, C₁₋₄alkyl substituted from 1 to 3 times by fluoro, and cycloalkyl, or R^{c1} is taken together with L¹² to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
n1 is 1 or 2;
z¹¹, z¹², z¹³ and z¹⁴ are independently 0 or 1; and
z¹⁵ and z¹⁶ are independently an integer from 0 to 4;
   provided at least one of z¹¹ and z¹³ is 1;
   or a salt thereof including a pharmaceutically acceptable salt thereof.

This invention also relates to pharmaceutically acceptable salts of the compounds of Formula (II).

Included in the compounds of the invention and used in the methods of the invention are compounds of Formula (III):

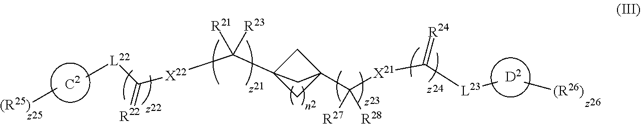

wherein:
L²² is selected from: a bond, —CH₂—, —NH—, —N(C₁₋₄alkyl)-, —N(C₁₋₄alkyl substituted from 1 to 3 times by fluoro)-, —O—, —CH₂—O—, —O—CH₂—, —O—CH₂—CH₂—, —CH₂—CH₂—O—, cyclopropyl, —O-cyclopropyl, cyclopropyl-O—, —CH₂-cyclopropyl, and cyclopropyl-CH₂—,
   or,
   L²² is taken together with R^{c2} to form:
   imidazolidinyl, imidazolidinyl-CH₂—, pyrrolidinyl, pyrrolidinyl-O—, pyrrolidinyl-NH—, pyrrolidinyl-CH₂—, oxopyrrolidinyl, oxopyrrolidinyl-O—, oxopyrrolidinyl-NH—, or oxopyrrolidinyl-CH₂—;
L²³ is selected from: a bond, —CH₂—, —NH—, —N(C₁₋₄alkyl)-, —N(C₁₋₄alkyl substituted from 1 to 3 times by fluoro)-, —O—, —CH₂—O—, —O—CH₂—, —O—CH₂—CH₂—, —CH₂—CH₂—O—, cyclopropyl, —O-cyclopropyl, cyclopropyl-O—, —CH₂-cyclopropyl, and cyclopropyl-CH₂—,
   or,
   L²³ is taken together with R^{b2} to form:
   imidazolidinyl, imidazolidinyl-CH₂—, pyrrolidinyl, pyrrolidinyl-O—, pyrrolidinyl-NH—, pyrrolidinyl-CH₂—, oxopyrrolidinyl, oxopyrrolidinyl-O—, oxopyrrolidinyl-NH—, or oxopyrrolidinyl-CH₂—;
R²¹ and R²³ are independently selected from: hydrogen, C₁₋₄alkyl, C₁₋₄alkyl substituted from 1 to 3 times by fluoro, or R²¹ and R²³ are taken together with the carbon to which they are attached to form cyclopropyl;
R²² and R²⁴ are independently O, or S;
R²⁵ is selected from: fluoro, chloro, bromo, iodo, C₁₋₄alkyl, —C(O)OC₁₋₄alkyl, —OH, —NH₂, —C(O)NHC₁₋₄alkyl, —OC₁₋₄alkyl, —OCH₂Ph, —C(O)Ph, —CF₃, —CN, —S(O)CH₃, —C(O)OH, —CONH₂, —NO₂, —C(O)CH₃, —CH₂C≡CH, —SCH₃, —SO₃H, —SO₂NH₂, —NHC(O)NH₂, —NHC(O)H, —NHOH, —OCF₃, —OCHF₂, —C(OH)R^{x1}R^{y1} (where R^{x1} is selected from hydrogen, C₁₋₄alkyl, and cycloalkyl, and R^{y1} is selected from C₁₋₄alkyl, and cycloalkyl);
R²⁶ is selected from: fluoro, chloro, bromo, iodo, C₁₋₄alkyl, —C(O)OC₁₋₄alkyl, —OH, —NH₂, —C(O)NHC₁₋₄alkyl, —OC₁₋₄alkyl, —OCH₂Ph, —C(O)Ph, —CF₃, —CN, —S(O)CH₃, —C(O)OH, —CONH₂, —NO₂, —C(O)CH₃, —CH₂Ca′CH, —SCH₃, —SO₃H, —SO₂NH₂, —NHC(O)NH₂, —NHC(O)H, —NHOH, —OCF₃, —OCHF₂, —C(OH)R^{x1}R^{y1} (where R^{x1} is selected from hydrogen, C₁₋₄alkyl, and cycloalkyl, and R^{y1} is selected from C₁₋₄alkyl, and cycloalkyl);
R²⁷ and R²⁸ are independently selected from: hydrogen, C₁₋₄alkyl, C₁₋₄alkyl substituted from 1 to 3 times by fluoro, or R²⁷ and R²⁸ are taken together with the carbon to which they are attached to form cyclopropyl;
C^{2′} and D^{2′} are each independently phenyl or pyridyl;
X²¹ is selected from: —O—, —NH—, and —NR^{b2}—;
   R^{b2} is selected from: C₁₋₄alkyl, C₁₋₄alkyl substituted from 1 to 3 times by fluoro, and cycloalkyl;
X²² is selected from: —O—, —NH—, and —NR^{c2}—;
   R^{c2} is selected from: C₁₋₄alkyl, C₁₋₄alkyl substituted from 1 to 3 times by fluoro, and cycloalkyl;
n2 is 1 or 2;
z²¹, z²², z²³ and z²⁴ are independently 0 or 1; and
z²⁵ and z²⁶ are independently an integer from 0 to 3;
   provided at least one of z²¹ and z²³ is 1;
   or a salt thereof including a pharmaceutically acceptable salt thereof.

This invention also relates to pharmaceutically acceptable salts of the compounds of Formula (III).

Included in the compounds of the invention are:
(3-(2-(4-Chlorophenoxy)acetamido)bicyclo[1.1.1]pentan-1-yl)methyl (4-chlorophenyl)carbamate;
4-Chlorophenyl ((3-(2-(4-chlorophenoxy)acetamido)bicyclo[1.1.1]pentan-1-yl)methyl)carbamate;
(3-(2-(4-chloro-3-fluorophenoxy)acetamido)bicyclo[1.1.1]pentan-1-yl)methyl (4-chlorophenyl)carbamate;
2-(4-Chlorophenoxy)-N-(3-((3-(4-chlorophenyl)ureido)methyl)bicyclo[1.1.1]pentan-1-yl)acetamide;
N,N′-(bicyclo[2.1.1]hexane-1,4-diylbis(methylene))bis(2-(4-chlorophenoxy)acetamide);
N,N′-(bicyclo[1.1.1]pentane-1,3-diylbis(methylene))bis(2-(4-chlorophenoxy)acetamide);
2-(4-Chlorophenoxy)-N-((3-(2-(4-chlorophenoxy)acetamido)bicyclo[1.1.1]pentan-1-yl)methyl)acetamide;
(R)-2-(4-chlorophenoxy)-N-(3-((4-(4-chlorophenoxy)-2-oxopyrrolidin-1-yl)methyl)bicyclo[1.1.1]pentan-1-yl)acetamide;
(3-(2-(4-chlorophenoxy)acetamido)bicyclo[1.1.1]pentan-1-yl)methyl (4-chloro-3-fluorophenyl)carbamate;
2-(4-chlorophenoxy)-N-(3-((2-(4-chlorophenyl)acetamido)methyl)bicyclo[1.1.1]pentan-1-yl)acetamide;
(3-(2-(4-chloro-3-fluorophenoxy)acetamido)bicyclo[1.1.1]pentan-1-yl)methyl (4-chloro-3-fluorophenyl)carbamate;
2-(4-chlorophenoxy)-N-(3-((3-(4-chlorophenyl)thioureido)methyObicyclo[1.1.1]pentan-1-yl)acetamide;
(3-(2-((5-chloropyridin-2-yl)oxy)acetamido)bicyclo[1.1.1]pentan-1-yOmethyl (4-chloro-3-fluorophenyl)carbamate;
(3-(2-(4-chloro-3-(trifluoromethyl)phenwry)acetamido)bicyclo[1.1.1]pentan-1-yl)methyl (4-chloro-3-fluorophenyl)carbamate;
(3-(2-(3,4-dichlorophenoxy)acetamido)bicyclo[1.1.1]pentan-1-yl)methyl (3,4-dichlorophenyl)carbamate;
(3-(2-(3,4-dichlorophenoxy)acetamido)bicyclo[1.1.1]pentan-1-yl)methyl (4-chloro-3-fluorophenyl)carbamate;
(3-(2-(4-chloro-3-fluorophenoxy)acetamido)bicyclo[1.1.1]pentan-1-yl)methyl (3,4-dichlorophenyl)carbamate;
(3-(2-(4-chloro-3-fluorophenoxy)acetamido)bicyclo[1.1.1]pentan-1-yl)methyl (4-bromophenyl)carbamate;
(3-(2-(3,4-dichlorophenoxy)acetamido)bicyclo[1.1.1]pentan-1-yOmethyl(4-chlorophenyl)carbamate;
(3-(2-(4-chloro-3-fluorophenoxy)acetamido)bicyclo[1.1.1]pentan-1-yl)methyl (4-chloro-3-methylphenyl)carbamate;
(3-(2-(4-fluorophenoxy)acetamido)bicyclo[1.1.1]pentan-1-yl)methyl (4-chloro-3-fluorophenyl)carbamate;
(3-(2-(3,4-difluorophenoxy)acetamido)bicyclo[1.1.1]pentan-1-yl)methyl (4-chloro-3-fluorophenyl)carbamate;
(3-(2-(3,4-difluorophenoxy)acetamido)bicyclo[1.1.1]pentan-1-yl)methyl (3,4-difluorophenyl)carbamate;
(3-(2-(4-fluorophenoxy)acetamido)bicyclo[1.1.1]pentan-1-yl)methyl (3,4-difluorophenyl)carbamate;
(3-(2-((4-chlorophenyl)amino)acetamido)bicyclo[1.1.1]pentan-1-yl)methyl (4-chloro-3-fluorophenyl)carbamate;
(3-(2,2-difluorobenzo[d][1,3]dioxole-5-carboxamido)bicyclo[1.1.1]pentan-1-yl)methyl (4-chloro-3-fluorophenyl)carbamate; now 19
(3-(6-chloro-4-methyl-3,4-dihydro-2H-benzo[b][1,4]oxazine-2-carboxamido)bicyclo[1.1.1]pentan-1-yl)methyl (4-chloro-3-fluorophenyl)carbamate;
(3-(6-chloro-3,4-dihydro-2H-benzo[b][1,4]oxazine-2-carboxamido)bicyclo[1.1.1]pentan-1-yl)methyl (4-chloro-3-fluorophenyl)carbamate;
2-(4-chlorophenoxy)-N-(3-((3-(3,4-dichlorophenyl)ureido)methyl)bicyclo[1.1.1]pentan-1-yl)acetamide;
2-(4-chloro-3-fluorophenoxy)-N-(3-((3-(3,4-dichlorophenyl)ureido)methyl)bicyclo[1.1.1]pentan-1-yl)acetamide;
2-(4-chlorophenoxy)-N-(3-((3-(4-chlorophenyl)-1-methylureido)methyl)bicyclo[1.1.1]pentan-1-yDacetamide;
(4-(2-(4-chlorophenoxy)acetamido)bicyclo[2.1.1]hexan-1-yl)methyl (4-chloro-3-fluorophenyl)carbamate; and (4-(2-(4-chloro-3-fluorophenoxy)acetamido)bicyclo[2.1.1]hexan-1-yl)methyl (4-chloro-3-fluorophenyl)carbamate;
   and salts thereof including pharmaceutically acceptable salts thereof.

Also included in the compounds of the invention are:
(3-(2-(4-Chlorophenoxy)acetamido)bicyclo[1.1.1]pentan-1-yl)methyl (4-chlorophenyl)carbamate;
4-Chlorophenyl ((3-(2-(4-chlorophenoxy)acetamido)bicyclo[1.1.1]pentan-1-yl)methyl)carbamate;
(3-(2-(4-chloro-3-fluorophenoxy)acetamido)bicyclo[1.1.1]pentan-1-yl)methyl (4-chlorophenyl)carbamate;
2-(4-Chlorophenoxy)-N-(3-((3-(4-chlorophenyl)ureido)methyl)bicyclo[1.1.1]pentan-1-yl)acetamide;
2-(4-Chlorophenoxy)-N-((3-(2-(4-chlorophenwry)acetamido)bicyclo[1.1.1]pentan-1-yl)methyl)acetamide;
(3-(2-(4-chlorophenoxy)acetamido)bicyclo[1.1.1]pentan-1-yl)methyl (4-chloro-3-fluorophenyl)carbamate;
(3-(2-(4-chloro-3-fluorophenoxy)acetamido)bicyclo[1.1.1]pentan-1-yl)methyl (4-chloro-3-fluorophenyl)carbamate;
2-(4-chlorophenoxy)-N-(3-((3-(4-chlorophenyl)thioureido)methyl)bicyclo[1.1.1]pentan-1-yl)acetamide;
(3-(2-((5-chloropyridin-2-yl)oxy)acetamido)bicyclo[1.1.1]pentan-1-yOmethyl (4-chloro-3-fluorophenyl)carbamate;
(3-(2-(4-chloro-3-(trifluoromethyl)phenwry)acetamido)bicyclo[1.1.1]pentan-1-yl)methyl (4-chloro-3-fluorophenyl)carbamate;
(3-(2-(3,4-dichlorophenoxy)acetamido)bicyclo[1.1.1]pentan-1-yl)methyl (3,4-dichlorophenyl)carbamate;
(3-(2-(3,4-dichlorophenoxy)acetamido)bicyclo[1.1.1]pentan-1-yl)methyl (4-chloro-3-fluorophenyl)carbamate;
(3-(2-(4-chloro-3-fluorophenoxy)acetamido)bicyclo[1.1.1]pentan-1-yl)methyl (3,4-dichlorophenyl)carbamate;
(3-(2-(4-chloro-3-fluorophenoxy)acetamido)bicyclo[1.1.1]pentan-1-yl)methyl (4-bromophenyl)carbamate;
(3-(2-(3,4-dichlorophenoxy)acetamido)bicyclo[1.1.1]pentan-1-yOmethyl(4-chlorophenyl)carbamate;
(3-(2-(4-chloro-3-fluorophenoxy)acetamido)bicyclo[1.1.1]pentan-1-yl)methyl (4-chloro-3-methylphenyl)carbamate;
(3-(2-(4-fluorophenoxy)acetamido)bicyclo[1.1.1]pentan-1-yl)methyl (4-chloro-3-fluorophenyl)carbamate;
(3-(2-(3,4-difluorophenoxy)acetamido)bicyclo[1.1.1]pentan-1-yl)methyl (4-chloro-3-fluorophenyl)carbamate;
(3-(2-(3,4-difluorophenoxy)acetamido)bicyclo[1.1.1]pentan-1-yl)methyl (3,4-difluorophenyl)carbamate;
(3-(2-(4-fluorophenoxy)acetamido)bicyclo[1.1.1]pentan-1-yl)methyl (3,4-difluorophenyl)carbamate;
(3-(2-((4-chlorophenyl)amino)acetamido)bicyclo[1.1.1]pentan-1-yl)methyl (4-chloro-3-fluorophenyl)carbamate;
(3-(2,2-difluorobenzo[d][1,3]dioxole-5-carboxamido)bicyclo[1.1.1]pentan-1-yl)methyl (4-chloro-3-fluorophenyl)carbamate;
(3-(6-chloro-4-methyl-3,4-dihydro-2H-benzo[b][1,4]oxazine-2-carboxamido)bicyclo[1.1.1]pentan-1-yl)methyl (4-chloro-3-fluorophenyl)carbamate;
(3-(6-chloro-3,4-dihydro-2H-benzo[b][1,4]oxazine-2-carboxamido)bicyclo[1.1.1]pentan-1-yl)methyl (4-chloro-3-fluorophenyl)carbamate;
2-(4-chlorophenoxy)-N-(3-((3-(3,4-dichlorophenyl)ureido)methyl)bicyclo[1.1.1]pentan-1-yl)acetamide;
2-(4-chloro-3-fluorophenoxy)-N-(3-((3-(3,4-dichlorophenyl)ureido)methyl)bicyclo[1.1.1]pentan-1-yl)acetamide;
2-(4-chlorophenoxy)-N-(3-((3-(4-chlorophenyl)-1-methylureido)methyl)bicyclo[1.1.1]pentan-1-yDacetamide;
(4-(2-(4-chlorophenoxy)acetamido)bicyclo[2.1.1]hexan-1-yl)methyl (4-chloro-3-fluorophenyl)carbamate; and (4-(2-(4-chloro-3-fluorophenoxy)acetamido)bicyclo[2.1.1]hexan-1-yl)methyl (4-chloro-3-fluorophenyl)carbamate;
   and salts thereof including pharmaceutically acceptable salts thereof.

Included in the compounds of the invention are:
(3-(2-(4-chlorophenoxy)acetamido)bicyclo[1.1.1]pentan-1-yl)methyl (4-chlorophenyl)carbamate;
4-chlorophenyl ((3-(2-(4-chlorophenoxy)acetamido)bicyclo[1.1.1]pentan-1-yl)methyl)carbamate;
(3-(2-(4-chloro-3-fluorophenoxy)acetamido)bicyclo[1.1.1]pentan-1-yl)methyl (4-chlorophenyl)carbamate;
2-(4-chlorophenoxy)-N-(3-((3-(4-chlorophenyl)ureido)methyl)bicyclo[1.1.1]pentan-1-yl)acetamide;
N,N′-(bicyclo[2.1.1]hexane-1,4-diylbis(methylene))bis(2-(4-chlorophenoxy)acetamide);
N,N′-(bicyclo[1.1.1]pentane-1,3-diylbis(methylene))bis(2-(4-chlorophenoxy)acetamide);
2-(4-chlorophenoxy)-N-((3-(2-(4-chlorophenoxy)acetamido)bicyclo[1.1.1]pentan-1-yl)methyl)acetamide;
(R)-2-(4-chlorophenoxy)-N-(3-((4-(4-chlorophenoxy)-2-oxopyrrolidin-1-yl)methyl)bicyclo[1.1.1]pentan-1-yl)acetamide;
(3-(2-(4-chlorophenoxy)acetamido)bicyclo[1.1.1]pentan-1-yl)methyl (4-chloro-3-fluorophenyl)carbamate;
2-(4-chlorophenoxy)-N-(3-((2-(4-chlorophenyl)acetamido)methyl)bicyclo[1.1.1]pentan-1-yl)acetamide;
(3-(2-(4-chloro-3-fluorophenoxy)acetamido)bicyclo[1.1.1]pentan-1-yl)methyl (4-chloro-3-fluorophenyl)carbamate;
2-(4-chlorophenoxy)-N-(3-((3-(4-chlorophenyl)thioureido)methyl)bicyclo[1.1.1]pentan-1-yl)acetamide;
(3-(2-((5-chloropyridin-2-yl)oxy)acetamido)bicyclo[1.1.1]pentan-1-yOmethyl (4-chloro-3-fluorophenyl)carbamate; and
(3-(2-(4-chloro-3-(trifluoromethyl)phenwry)acetamido)bicyclo[1.1.1]pentan-1-yl)methyl (4-chloro-3-fluorophenyl)carbamate;
   and salts thereof including pharmaceutically acceptable salts thereof.

Also included in the compounds of the invention are:
(3-(2-(4-chlorophenoxy)acetamido)bicyclo[1.1.1]pentan-1-yl)methyl (4-chlorophenyl)carbamate;
4-chlorophenyl ((3-(2-(4-chlorophenoxy)acetamido)bicyclo[1.1.1]pentan-1-yl)methyl)carbamate;
(3-(2-(4-chloro-3-fluorophenoxy)acetamido)bicyclo[1.1.1]pentan-1-yl)methyl (4-chlorophenyl)carbamate;
2-(4-chlorophenoxy)-N-(3-((3-(4-chlorophenyl)ureido)methyl)bicyclo[1.1.1]pentan-1-yl)acetamide;
N,N′-(bicyclo[2.1.1]hexane-1,4-diylbis(methylene))bis(2-(4-chlorophenoxy)acetamide);
N,N′-(bicyclo[1.1.1]pentane-1,3-diylbis(methylene))bis(2-(4-chlorophenoxy)acetamide); and
2-(4-chlorophenoxy)-N-((3-(2-(4-chlorophenoxy)acetamido)bicyclo[1.1.1]pentan-1-yl)methyl)acetamide;
   and salts thereof including pharmaceutically acceptable salts thereof.

Also included in the compounds of the invention are:
(R)-2-(4-chlorophenoxy)-N-(3-((4-(4-chlorophenoxy)-2-oxopyrrolidin-1-yl)methyl)bicyclo[1.1.1]pentan-1-yl)acetamide;
(3-(2-(4-chlorophenoxy)acetamido)bicyclo[1.1.1]pentan-1-yl)methyl (4-chloro-3-fluorophenyl)carbamate;
2-(4-chlorophenoxy)-N-(3-((2-(4-chlorophenyl)acetamido)methyl)bicyclo[1.1.1]pentan-1-yl)acetamide;
(3-(2-(4-chloro-3-fluorophenoxy)acetamido)bicyclo[1.1.1]pentan-1-yl)methyl (4-chloro-3-fluorophenyl)carbamate;
2-(4-chlorophenoxy)-N-(3-((3-(4-chlorophenyl)thioureido)methyl)bicyclo[1.1.1]pentan-1-yl)acetamide;
(3-(2-((5-chloropyridin-2-yl)oxy)acetamido)bicyclo[1.1.1]pentan-1-yOmethyl (4-chloro-3-fluorophenyl)carbamate; and
(3-(2-(4-chloro-3-(trifluoromethyl)phenoxy)acetamido)bicyclo[1.1.1]pentan-1-yl)methyl (4-chloro-3-fluorophenyl)carbamate;
   and salts thereof including pharmaceutically acceptable salts thereof.

To clarify the obvious intent, in any of the above Formulas, when “z” in a

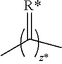

moiety is 0, and the adjacent “R*” and “L*” moieties form a ring, such as a heterocycloalkyl, for example a pyrrolidinyl, the “R*” and “L*” moieties do not have to be adjacent in the ring.
Further, in any of the above Formulas, in any

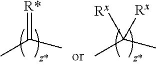

moiety, it is understood that the “R*” or “R^{x}”s will be absent whenever “Z*” is 0.
Further, in any of the above Formulas, in a

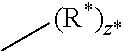

moiety, it is understood that whenever “z*” is 0, any substituent that could be an “R*” group, will be hydrogen.
Further, in the above Formulas, R⁵, R⁶, R¹⁵, R¹⁶, R²⁵, R²⁶, R^{5′}, R^{6′}, R^{15′}, R^{16′}, R^{25′}, R^{26′}, R^{35′}, and R^{26′}, are indicated by: is or are “selected from . . . ”. To clarify the obvious intent, for these “R” structures, when two of the same groups are on the same compound, (for example when two R⁵ groups are on the same compound), each R⁵ can be a different substituent. For Example, one R⁵ can be F and the other R⁵ can be Cl.
In embodiments, R^{5′} is selected from: fluoro, chloro, bromo, iodo, —OCH₃, —OCH₂Ph, —C(O)Ph, —CF₃, —CN, —S(O)CH₃, —OH, —NH₂, —COOH, —CONH₂, —NO₂, —C(O)CH₃, —CH₂C≡CH, —SO₃H, —SO₂NH₂, —NHC(O)NH₂, —SCH₃, —NHC(O)H, —NHOH, —OCH₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁵ is independently fluoro, chloro, bromo, iodo, —OCH₃, —OCH₂Ph, —CH₃, —OH, —CF₃, —CN, —S(O)CH₃, —NO₂, —C(O)CH₃, —C(O)Ph, —CH(CH₃)₂, or —CH. CH. In embodiments, R⁵ is —F. In embodiments, R⁵ is —Cl. In embodiments, R^{5′} is —Br. In embodiments, R^{5′} is —I. In embodiments, R^{5′} is substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁵ is unsubstituted C₁₋₆alkyl, unsubstituted heteroalkyl, unsubstituted cycloalkyl, unsubstituted heterocycloalkyl, unsubstituted aryl, or unsubstituted heteroaryl. In embodiments, R^{5′} is —OCH₃. In embodiments, R^{5′} is —OCH₂Ph. In embodiments, R^{5′} is —CH₃. In embodiments, R^{5′} is —OH. In embodiments, R^{5′} is —CF₃. In embodiments, R^{5′} is —CN. In embodiments, R^{5′} is —S(O)CH₃. In embodiments, R^{5′} is —NO₂. In embodiments, R^{5′} is —C(O)CH₃. In embodiments, R^{5′} is —C(O)Ph. In embodiments, R^{5′} is —CH(CH₃)₂. In embodiments, R^{5′} is —C≡CH. In embodiments, R^{5′} is —CH₂C≡CH. In embodiments, R^{5′} is —SO₃H. In embodiments, R^{5′} is —SO₂NH₂. In embodiments, R^{5′} is —NHC(O)NH₂. In embodiments, R^{5′} is —NHC(O)H. In embodiments, R^{5′} is —NHOH. In embodiments, R^{5′} is —OCF₃. In embodiments, R^{5′} is —OCHF₂.

In embodiments, R^{6′} is selected from: fluoro, chloro, bromo, iodo, —OCH₃, —OCH₂Ph, —C(O)Ph, —CF₃, —CN, —S(O)CH₃, —OH, —NH₂, —COOH, —CONH₂, —NO₂, —SCH₃, —C(O)CH₃, —C≡CH, —CH₂C≡CH, —SO₃H, —SO₂NH₂, —NHC(O)NH₂, —NHC(O)H, —NHOH, —OCF₃, —OCHF₂, substituted or unsubstituted C₁₋₆alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁶ is independently fluoro, chloro, bromo, iodo, —OCH₃, —OCH₂Ph, —CH₃, —OH, —CF₃, —CN, —S(O)CH₃, —NO₂, —C(O)CH₃, —C(O)Ph, —CH(CH₃)₂, or —C≡CH. In embodiments, R^{6′} is —F. In embodiments, R^{6′} is —Cl. In embodiments, R^{6′} is —Br. In embodiments, R^{6′} is —I. In embodiments, R^{6′} is substituted or unsubstituted C₁₋₆alkyl, substituted or unsubstituted C₁₋₆heteroalkyl, substituted or unsubstituted cycloalkyl, substituted or unsubstituted heterocycloalkyl, substituted or unsubstituted aryl, or substituted or unsubstituted heteroaryl. In embodiments, R^{6′} is unsubstituted C₁₋₆alkyl, unsubstituted C₁₋₆heteroalkyl, unsubstituted cycloalkyl, unsubstituted heterocycloalkyl, unsubstituted aryl, or unsubstituted heteroaryl. In embodiments, R^{6′} is —OCH₃. In embodiments, R^{6′} is —OCH₂Ph. In embodiments, R^{6′} is —CH₃. In embodiments, R^{6′} is —OH. In embodiments, R^{6′} is —CF₃. In embodiments, R^{6′} is —CN. In embodiments, R^{6′} is —S(O)CH₃. In embodiments, R^{6′} is —NO₂. In embodiments, R^{6′} is —C(O)CH₃. In embodiments, R^{6′} is —C(O)Ph. In embodiments, R^{6′} is —CH(CH₃)₂. In embodiments, R^{6′} is —C≡CH. In embodiments, R^{6′} is —CH₂C≡CH. In embodiments, R^{6′} is —SO₃H. In embodiments, R^{6′} is —SO₂NH₂. In embodiments, R^{6′} is —NHC(O)NH₂. In embodiments, R^{6′} is —NHC(O)H. In embodiments, R^{6′} is —NHOH. In embodiments, R^{6′} is —OCF₃. In embodiments, R^{6′} is —OCHF₂.

In embodiments, R^{2′} is NR^{a}. In embodiments, R^{2′} is NH. In embodiments, R^{2′} is O. In embodiments, R^{2′} is S. In embodiments, R^{2′} is CH₂. In embodiments, R^{4′} is NR^{a}. In embodiments, R^{4′} is NH. In embodiments, R^{4′} is 0. In embodiments, R^{4′} is S. In embodiments, R^{4′} is CH₂. In embodiments, R^{2′} and R^{4′} are NH. In embodiments, R^{2′} and R^{4′} are O. In embodiments, R^{2′} and R^{4′} are S. In embodiments, R^{2′} and R^{4′} are NR^{a}.

In embodiments, R^{7′} is selected from: C₁₋₄alkyl and hydrogen. In embodiments, R^{7′} is C₁₋₄alkyl. In embodiments, R^{7′} is hydrogen. In embodiments, R^{17′} is selected from: C₁₋₄alkyl and hydrogen. In embodiments, R^{17′} is C₁₋₄alkyl. In embodiments, R^{17′} is hydrogen. In embodiments, R^{27′} is selected from: C₁₋₄alkyl and hydrogen. In embodiments, R^{27′} is C₁₋₄alkyl. In embodiments, R^{27′} is hydrogen.

In embodiments, R^{8′} is selected from: C₁₋₄alkyl and hydrogen. In embodiments, R^{8′} is C₁₋₄alkyl. In embodiments, R^{8′} is hydrogen. In embodiments, R^{18′} is selected from: C₁₋₄alkyl and hydrogen. In embodiments, R^{18′} is C₁₋₄alkyl. In embodiments, R^{18′} is hydrogen. In embodiments, R^{28′} is selected from: C₁₋₄alkyl and hydrogen. In embodiments, R^{28′} is Cl-4alkyl. In embodiments, R^{28′} is hydrogen.

In embodiments, L^{2′} is a bond. In embodiments, L^{2′} is a substituted or unsubstituted C₁₋₆alkylene. In embodiments, L^{2′} is a substituted or unsubstituted C₁₋₆heteroalkylene. In embodiments, L^{2′} is a bond, —O—, —S—, —NH—, —S(O)—, or —S(O)₂—. In embodiments, L^{2′} is a bond or substituted or unsubstituted C₁₋₆alkylene. In embodiments, L^{2′} is a bond, —O—, or —NH—. In embodiments, L^{2′} is a bond. In embodiments, L^{2′} is —O—. In embodiments, L^{2′} is —S—. In embodiments, L^{2′} is —NH—. In embodiments, L^{2′} is —S(O)—. In embodiments, L^{2′} is —S(O)₂—. In embodiments, L^{2′} is a substituted or unsubstituted C₁-C₅heteroalkylene. In embodiments, L^{2′} is an unsubstituted C₁-C₅heteroalkylene. In embodiments, L^{2′} is a substituted or unsubstituted C₁-C₄heteroalkylene. In embodiments, L^{2′} is an unsubstituted C₁-C₄heteroalkylene. In embodiments, L^{2′} is a substituted or unsubstituted C₁-C₃heteroalkylene. In embodiments, L^{2′} is an unsubstituted C₁-C₃ heteroalkylene. In embodiments, L^{2′} is a substituted C₁-C₅heteroalkylene. In embodiments, L^{2′} is a substituted C₁-C₆heteroalkylene. In embodiments, L^{2′} is a substituted C₁-C₄heteroalkylene. In embodiments, L^{2′} is a C₁-C₆heteroalkylene substituted with —CF₃. In embodiments, L^{2′} is cyclopropyl. In embodiments, L^{2′} is —CH₂-cycloalkyl. In embodiments, L^{2′} is cycloalkyl-CH₂—.

In embodiments, L^{3′} is a bond. In embodiments, L^{3′} is a substituted or unsubstituted C₁₋₆alkylene. In embodiments, L^{3′} is a substituted or unsubstituted C₁₋₆heteroalkylene. In embodiments, L^{3′} is a bond, —O—, —S—, —NH—, —S(O)—, or —S(O)₂—. In embodiments, L^{3′} is a bond or substituted or unsubstituted C₁₋₆alkylene. In embodiments, L^{3′} is a bond, —O—, or —NH—. In embodiments, L^{3′} is a bond. In embodiments, L^{3′} is —O—. In embodiments, L^{3′} is —S—. In embodiments, L^{3′} is —NH—. In embodiments, L^{3′} is —S(O)—. In embodiments, L^{3′} is —S(O)₂—. In embodiments, L^{3′} is a substituted or unsubstituted C₁-C₅heteroalkylene. In embodiments, L^{3′} is an unsubstituted C₁-C₅heteroalkylene. In embodiments, L^{3′} is a substituted or unsubstituted C₁-C₄heteroalkylene. In embodiments, L^{3′} is an unsubstituted C₁-C₄heteroalkylene. In embodiments, L^{3′} is a substituted or unsubstituted C₁-C₃heteroalkylene. In embodiments, L^{3′} is an unsubstituted C₁-C₃ heteroalkylene. In embodiments, L^{3′} is a substituted C₁-C₅heteroalkylene. In embodiments, L^{3′} is a substituted C₁-C₆heteroalkylene. In embodiments, L^{3′} is a substituted C1-C4heteroalkylene. In embodiments, L^{3′} is a C₁-C₆heteroalkylene substituted with —CF₃. In embodiments, L^{3′} is cyclopropyl. In embodiments, L^{3′} is —CH₂-cycloalkyl. In embodiments, L^{3′} is cycloalkyl-CH₂—.
In embodiments, L^{3′} is taken together with R^{b} to form heterocycloalkyl. Suitably the heterocycloalkyl is imidazolidinyl or pyrrolidinyl. Suitably the heterocycloalkyl is imidazolidinyl. Suitably the heterocycloalkyl is pyrrolidinyl.
In embodiments, L^{2′} is taken together with R^{c} to form heterocycloalkyl. Suitably the heterocycloalkyl is imidazolidinyl or pyrrolidinyl. Suitably the heterocycloalkyl is imidazolidinyl. Suitably the heterocycloalkyl is pyrrolidinyl.
In embodiments, L^{12′} is taken together with R^{c1} to form heterocycloalkyl. Suitably the heterocycloalkyl is imidazolidinyl or pyrrolidinyl. Suitably the heterocycloalkyl is imidazolidinyl. Suitably the heterocycloalkyl is pyrrolidinyl.
In embodiments, L^{13′} is taken together with R^{b1} to form heterocycloalkyl. Suitably the heterocycloalkyl is imidazolidinyl or pyrrolidinyl. Suitably the heterocycloalkyl is imidazolidinyl. Suitably the heterocycloalkyl is pyrrolidinyl.
In embodiments, the symbol z^{2′} is 0. In embodiments, the symbol z^{2′} is 1. In embodiments, the symbol z^{4′} is 0. In embodiments, the symbol z^{4′} is 1. In embodiments, the symbols z² and z^{4′} are 0. In embodiments, the symbols z^{2′} and z^{4′} are 1. In embodiments, the symbol z^{5′} is 0. In embodiments, the symbol z^{5′} is 1. In embodiments, the symbol z^{5′} is 2. In embodiments, the symbol z^{5′} is 3. In embodiments, the symbol z^{5′} is 4. In embodiments, the symbol z^{6′} is 0. In embodiments, the symbol z^{6′} is 1. In embodiments, the symbol z^{6′} is 2. In embodiments, the symbol z^{6′} is 3. In embodiments, the symbol z^{6′} is 4. In embodiments, the symbol z^{6′} is 0. In embodiments, the symbol z^{6′} is 1.

The skilled artisan will appreciate that salts, including pharmaceutically acceptable salts, of the compounds according to Formula (X) may be prepared. Indeed, in certain embodiments of the invention, salts including pharmaceutically-acceptable salts of the compounds according to Formula (X) may be preferred over the respective free or unsalted compound. Accordingly, the invention is further directed to salts, including pharmaceutically-acceptable salts, of the compounds according to Formula (X).

The salts, including pharmaceutically acceptable salts, of the compounds of the invention are readily prepared by those of skill in the art.

Typically, the salts of the present invention are pharmaceutically acceptable salts. Salts encompassed within the term “pharmaceutically acceptable salts” refer to non-toxic salts of the compounds of this invention.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

The compounds according to Formula (X) may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may be present in a substituent such as an alkyl group. Where the stereochemistry of a chiral center present in a compound of Formula (X), or in any chemical structure illustrated herein, if not specified the structure is intended to encompass all individual stereoisomers and all mixtures thereof. Thus, compounds according to Formula (X) containing one or more chiral centers may be used as racemic mixtures, enantiomerically or diastereomerically enriched mixtures, or as enantiomerically or diastereomerically pure individual stereoisomers.

The compounds according to Formula (X) and pharmaceutically acceptable salts thereof may contain isotopically-labelled compounds, which are identical to those recited in Formula (X) and following, but for the fact that one or more atoms are replaced by an atom having an atomic mass or mass number different from the atomic mass or mass number usually found in nature. Examples of such isotopes include isotopes of hydrogen, carbon, nitrogen, oxygen, phosphorous, sulphur, fluorine, iodine, and chlorine, such as 2H, 3H, 11C, 13C, 14C, 15N, 17O, 18O, 31P, 32P, 35S, 18F, 36Cl, 123I and 125I.

Isotopically-labelled compounds, for example those into which radioactive isotopes such as 3H or 14C are incorporated, are useful in drug and/or substrate tissue distribution assays. Tritiated, i.e., 3H, and carbon-14, i.e., 14C, isotopes are particularly preferred for their ease of preparation and detectability. 11C and 18F isotopes are particularly useful in PET (positron emission tomography), and 125I isotopes are particularly useful in SPECT (single photon emission computerized tomography), both are useful in brain imaging. Further, substitution with heavier isotopes such as deuterium, i.e., 2H, can afford certain therapeutic advantages resulting from greater metabolic stability, for example increased in vivo half-life or reduced dosage requirements and, hence, may be preferred in some circumstances. Isotopically labelled compounds can generally be prepared by substituting a readily available isotopically labelled reagent for a non-isotopically labelled reagent.

The compounds according to Formula (X) may also contain double bonds or other centers of geometric asymmetry. Where the stereochemistry of a center of geometric asymmetry present in Formula (X), or in any chemical structure illustrated herein, is not specified, the structure is intended to encompass the trans (E) geometric isomer, the cis (Z) geometric isomer, and all mixtures thereof. Likewise, all tautomeric forms are also included in Formula (X) whether such tautomers exist in equilibrium or predominately in one form.

The compounds of Formula (X) or salts, including pharmaceutically acceptable salts, thereof may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically acceptable solvates may be formed wherein solvent molecules are incorporated into the crystalline lattice during crystallization. Solvates wherein water is the solvent that is incorporated into the crystalline lattice are typically referred to as “hydrates.” Hydrates include stoichiometric hydrates as well as compositions containing vaiable amounts of water.

The skilled artisan will further appreciate that certain compounds of Formula (X) or salts, including pharmaceutically acceptable salts thereof that exist in crystalline form, including the various solvates thereof, may exhibit polymorphism (i.e. the capacity to occur in different crystalline structures). These different crystalline forms are typically known as “polymorphs.” Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

While aspects for each variable have generally been listed above separately for each variable this invention includes those compounds in which several or each aspect in Formula (I) is selected from each of the aspects listed above. Therefore, this invention is intended to include all combinations of aspects for each variable.

### Definitions

“Alkyl” and “alkylene”, and derivatives thereof, refer to a hydrocarbon chain having the specified number of “member atoms”. Alkyl being monovalent and alkylene being bivalent. For example, C₁-C₆ alkyl refers to an alkyl group having from 1 to 6 member atoms. Alkyl and alkylene groups may be saturated, unsaturated, straight or branched. Representative branched alkyl groups have one, two, or three branches. Alkyl and alkylene include: methyl, ethyl, ethylene, propyl (n-propyl and isopropyl), butene, butyl (n-butyl, isobutyl, and t-butyl), pentyl and hexyl.
“Aryl” refers to an aromatic hydrocarbon ring. Aryl groups are monocyclic, bicyclic, and tricyclic ring systems having a total of five to fourteen ring member atoms, wherein at least one ring system is aromatic and wherein each ring in the system contains 3 to 7 member atoms, such as phenyl, naphthalene, tetrahydronaphthalene and biphenyl. Suitably aryl is phenyl.
“Cycloalkyl”, unless otherwise defined, refers to a saturated or unsaturated non aromatic hydrocarbon ring having from three to seven carbon atoms. Cycloalkyl groups are monocyclic ring systems. For example, C₃-C₇ cycloalkyl refers to a cycloalkyl group having from 3 to 7 member atoms. Examples of cycloalkyl as used herein include: cyclopropyl, cyclobutyl, cyclopentyl, cyclohexyl, cyclobutenyl, cyclopentenyl, cyclohexenyl and cycloheptyl. Suitably cycolalkyl is selected from: cyclopropyl, cyclobutyl and cyclohexyl. Suitably “cycloalkyl” is cyclopropyl. Suitably “cyloalkyl” is cyclobutyl.
“Halo” refers to fluoro, chloro, bromo, and iodo.
“Heteroaryl” refers to a monocyclic aromatic 4 to 8 member ring containing 1 to 7 carbon atoms and containing 1 to 4 heteroatoms, provided that when the number of carbon atoms is 3, the aromatic ring contains at least two heteroatoms, or to such aromatic ring is fused one or more rings, such as heteroaryl rings, aryl rings, heterocyclic rings, or cycloalkyl rings. Heteroaryl groups containing more than one heteroatom may contain different heteroatoms. Heteroaryl includes but is not limited to: benzoimidazolyl, benzothiazolyl, benzothiophenyl, benzopyrazinyl, benzotriazolyl, benzotriazinyl, benzo[1,4]dioxanyl, benzofuranyl, 9H-a-carbolinyl, cinnolinyl, furanyl, pyrazolyl, imidazolyl, indolizinyl, naphthyridinyl, oxazolyl, oxothiadiazolyl, oxadiazolyl, phthalazinyl, pyridyl, pyrrolyl, purinyl, pteridinyl, phenazinyl, pyrazinyl, pyrazolopyrimidinyl, pyrazolopyridinyl, pyrrolizinyl, pyrimidyl, isothiazolyl, furazanyl, pyrimidinyl, tetrazinyl, isoxazolyl, quinoxalinyl, quinazolinyl, quinolinyl, quinolizinyl, thienyl, thiophenyl, triazolyl, triazinyl, tetrazolopyrimidinyl, triazolopyrimidinyl, tetrazolyl, thiazolyl and thiazolidinyl. Suitably heteroaryl is selected from: pyrazolyl, imidazolyl, oxazolyl and thienyl. Suitably heteroaryl is a pyridyl group or an imidazolyl group. Suitably heteroaryl is pyridyl or pyrazinyl. Suitably heteroaryl is pyridyl.
“Heterocycloalkyl” refers to a saturated or unsaturated non-aromatic ring containing 4 to 12 member atoms, of which 1 to 11 are carbon atoms and from 1 to 6 are heteroatoms. Heterocycloalkyl groups containing more than one heteroatom may contain different heteroatoms. Heterocycloalkyl groups are monocyclic ring systems or a monocyclic ring fused with an aryl ring or to a heteroaryl ring having from 3 to 6 member atoms.
Heterocycloalkyl includes: pyrrolidinyl, tetrahydrofuranyl, dihydrofuranyl, pyranyl, tetrahydropyranyl, dihydropyranyl, tetrahydrothienyl, pyrazolidinyl, oxazolidinyl, imidazolidinyl, oxetanyl, thiazolidinyl, piperidinyl, homopiperidinyl, piperazinyl, morpholinyl, thiamorpholinyl, 1,3-dioxolanyl, 1,3-dioxanyl, 1,4-dioxanyl, 1,3-oxathiolanyl, 1,3-oxathianyl, 1,3-dithianyl, 1,3oxazolidin-2-one, hexahydro-1H-azepin, 4,5,6,7,tetrahydro-1H-benzimidazol, piperidinyl, 1,2,3,6-tetrahydro-pyridinyl and azetidinyl. Suitably, “heterocycloalkyl” includes: piperidinyl, tetrahydrofuranyl, tetrahydropyranyl, imidazolidinyl, oxetanyl, and pyrrolidinyl. Suitably, “heterocycloalkyl” is selected from: imidazolidinyl, tetrahydropyranyl and pyrrolidinyl.
Suitably, “heterocycloalkyl” is selected from: imidazolidinyl, tetrahydropyranyl, pyrrolidinyl, 1,4-dioxanyl, tetrahydropyranyl, or 1,4-oxazinyl.
“Heteroatom” refers to a nitrogen, sulfur or oxygen atom.
“Heteroalkyl” and “heteroalkylene” by itself or in combination with another term, means, unless otherwise stated, a non-cyclic stable straight or branched chain, or combinations thereof, including at least one carbon atom (and up to the number specified) and at least one heteroatom selected from the group consisting of O, N, P, Si, and S, and wherein the nitrogen and sulfur atoms may optionally be oxidized, and the nitrogen heteroatom may optionally be quaternized. For example, C₁₋₆heteroalkyl(ene) contains at least one and up to 6 carbon atoms, in addition to at least one heteroatom. Heteroalkyl being monovalent and heteroalkylene being bivalent. The heteroalkyl and heteroalkylene groups may be taken together with another substituent to form a heterocycloalkyl group. The heteroatom(s) 0, N, P, S, and Si may be placed at any interior position of the heteroalkyl or heteroalkylene group or at the position at which the alkyl group is attached to the remainder of the molecule. Heteroalkyl examples include, but are not limited to: —CH₂—CH₂—O—CH₃, —CH₂—CH₂—NH—CH₃, —CH₂—CH₂—N(CH₃)₂, —CH₂—S—CH₂—CH₃, —S(O)—CH₃, —CH₂—CH₂—S(O)₂—CH₃, —CH═CH—O—CH₃, —Si(CH₃)₃, —CH₂—CH═N—OCH₃, —CH═CHN(CH₃)₂, —O—CH₃, —O—CH₂—CH₃, —CN. Heteroalkylene examples include, but are not limited to: —CH₂—CH₂—O—CH₂—, —CH₂—CH₂—NH—CH₂—, —CH₂—CH₂—N(CH₃)CH₂—, —CH₂—S—CH₂—CH₂—, —S(O)—CH₂—, —CH₂—CH₂—S(O)₂—CH₂—, —CH═CH—O—CH₂—, —Si(CH₃)₂CH₂—, —N(CH₃)CH₂—, —O—CH₂—CH₂—CH₂—, —CH₂—CH═N—OCH₂—, —CH═CHN(CH₃)CH₂—, —O—CH₂—, and —O—CH₂—CH₂—. Up to two or three heteroatoms may be consecutive, such as, for example, —CH₂—NH—OCH₃ and —CH₂—O—Si(CH₃)₃.

For the avoidance of doubt and in order to clarify the obvious chemical intent, where substituent groups are specified by their conventional chemical formulae, written from left to right, they equally encompass the chemically identical substituents that would result from writing the structure in reverse or to put it another way, from right to left (for example, —CHzO— is equivalent to —OCHz-), unless the chemically identical substituent written in the reverse is also specified.

For the avoidance of doubt, the structure

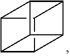

as used herein, refers to cubane.

The term “imidazolidinyl” as used herein, unless otherwise indicated, is meant a compound of the structure,

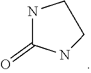

“Substituted” as used herein, unless otherwise defined, is meant that the subject chemical moiety has from one to nine substituents, suitably from one to five substituents, selected from the group consisting of:
fluoro,
chloro,
bromo,
iodo,
C₁₋₆alkyl,
C₁₋₆alkyl substituted with from 1 to 6 substituents independently selected from: fluoro, oxo, —OH, —COOH, —NH₂, and —CN,
—OC₁₋₆alkyl,
—OC₁₋₆alkyl substituted with from 1 to 6 substituents independently selected from: fluoro, oxo, —OH, —COOH, —NH₂, and —CN,
mercapto,
—SR^{x},
   where R^{x} is selected from C₁₋₆alkyl, and C₁₋₆alkyl substituted with from 1 to 6 substituents independently selected from: fluoro, oxo, —OH, —COOH, —NH₂, and —CN,
—S(O)R^{x},
   where R^{x} is selected from C₁₋₆alkyl, and C₁₋₆alkyl substituted with from 1 to 6 substituents independently selected from: fluoro, oxo, —OH, —COOH, —NH₂, and —CN,
—S(O)₂H,
—S(O)₂R^{x},
   where R^{x} is selected from C₁₋₆alkyl, and C₁₋₆alkyl substituted with from 1 to 6 substituents independently selected from: fluoro, oxo, —OH, —COOH, —NH₂, and —CN,
oxo,
hydroxy,
amino,
—NHR^{x},
   where R^{x} is selected from C₁₋₆alkyl, and C₁₋₆alkyl substituted with from 1 to 6 substituents independently selected from: fluoro, oxo, —OH, —COOH, —NH₂, and —CN,
—NR^{x1}R^{x2},
   where R^{x1} and R^{x2} are each independently selected from C₁₋₆alkyl, and C₁₋₆alkyl substituted with from 1 to 6 substituents independently selected from: fluoro, oxo, —OH, —COOH, —NH₂, and —CN,
guanidino,
—C(O)OH,
—C(O)OR^{x},
   where R^{x} is selected from C₁₋₆alkyl, and C₁₋₆alkyl substituted with from 1 to 6 substituents independently selected from: fluoro, oxo, —OH, —COOH, —NH₂, and —CN,
—C(O)NH₂,
—C(O)NHR^{x},
   where R^{x} is selected from C₁₋₆alkyl, and C₁₋₆alkyl substituted with from 1 to 6 substituents independently selected from: fluoro, oxo, —OH, —COOH, —NH₂, and —CN,
—C(O)NR^{x1}R^{x2},
   where R^{x1} and R^{x2} are each independently selected from C₁₋₆alkyl, and C₁₋₆alkyl substituted with from 1 to 6 substituents independently selected from: fluoro, oxo, —OH, —COOH, —NH₂, and —CN,
—S(O)₂NH₂,
—S(O)₂NHR^{x},
   where R^{x} is selected from C₁₋₆alkyl, and C₁₋₆alkyl substituted with from 1 to 6 substituents independently selected from: fluoro, oxo, —OH, —COOH, —NH₂, and —CN,
—S(O)₂NR^{x1}R^{x2},
   where R^{x1} and R^{x2} are each independently selected from C₁₋₆alkyl, and C₁₋₆alkyl substituted with from 1 to 6 substituents independently selected from: fluoro, oxo, —OH, —COOH, —NH₂, and —CN, —NHS(O)₂H,
—NHS(O)₂R^{x},
   where R^{x} is selected from C₁₋₆alkyl, and C₁₋₆alkyl substituted with from 1 to 6 substituents independently selected from: fluoro, oxo, —OH, —COOH, —NH₂, and —CN,
—NHC(O)H,
—NHC(O)R^{x},
   where R^{x} is selected from C₁₋₆alkyl, and C₁₋₆alkyl substituted with from 1 to 6 substituents independently selected from: fluoro, oxo, —OH, —COOH, —NH₂, and —CN,
—NHC(O)NH₂,
—NHC(O)NHR^{x},
   where R^{x} is selected from C₁₋₆alkyl, and C₁₋₆alkyl substituted with from 1 to 6 substituents independently selected from: fluoro, oxo, —OH, —COOH, —NH₂, and —CN,
—NHC(O)NR^{x1}R^{x2},
   where R^{x1} and R^{x2} are each independently selected from C₁₋₆alkyl, and C₁₋₆alkyl substituted with from 1 to 6

Substituents independently selected from: fluoro, oxo, —OH, —COOH, —NH₂, and —CN,
nitro, and
cyano.
   Suitably “substituted” means the subject chemical moiety has from one to four substituents selected from the group consisting of:
fluoro,
chloro,
bromo,
iodo,
C₁₋₄alkyl,
C₁₋₄alkyl substituted with from 1 to 4 substituents independently selected from: fluoro, oxo, —OH, —COOH, —NH₂, and —CN,
—OC₁₋₄alkyl,
—OC₁₋₄alkyl substituted with from 1 to 4 substituents independently selected from: fluoro, oxo, —OH, —COOH, —NH₂, and —CN,
—SH,
—S(O)₂H,
oxo,
hydroxy,
amino,
—NHR^{x},
   where R^{x} is selected from C₁₋₄alkyl, and C₁₋₆alkyl substituted one to 4 times by fluoro,
—NR^{x1}R^{x2},
   where R^{x1} and R^{x2} are each independently selected from C₁₋₄alkyl, and C₁₋₄alkyl substituted one to four times by fluoro,
guanidino,
—C(O)OH,
—C(O)OR^{x},
   where R^{x} is selected from C₁₋₄alkyl, and C₁₋₄alkyl substituted one to four times by fluoro,
—C(O)NH₂,
—C(O)NHR^{x},
   where R^{x} is selected from C₁₋₄alkyl, and C₁₋₄alkyl substituted one to four times by fluoro,
—C(O)NR^{x1}R^{x2},
   where R^{x1} and R^{x2} are each independently selected from C₁₋₄alkyl, and C₁₋₄alkyl substituted one to four times by fluoro,
—S(O)₂NH₂,
—NHS(O)₂H,
—NHC(O)H,
—NHC(O)NH₂,
nitro, and
cyano.
   Suitably “substituted” means the subject chemical moiety has from one to four substituents selected from the group consisting of:
fluoro,
chloro,
bromo,
iodo,
C₁₋₄alkyl,
C₁₋₄alkyl substituted with from 1 to 4 substituents independently selected from: fluoro, oxo, —OH, —COOH, —NH₂, —NHC₁₋₃alkyl, —N(C₁₋₃alkyl)₂, —OC₁₋₄alkyl and —CN,
—OC₁₋₄alkyl,
—OC₁₋₄alkyl substituted with from 1 to 4 substituents independently selected from: fluoro, oxo, —OH, —COOH, —NH₂, —NHC₁₋₃alkyl, —N(C₁₋₃alkyl)₂, and —CN,
—SH,
—S(O)₂H,
oxo,
hydroxy,
amino,
—NHR^{x},
   where R^{x} is selected from C₁₋₄alkyl, and C₁₋₄alkyl substituted one to 4 times by fluoro,
—NR^{x1}R^{x2},
   where R^{x1} and R^{x2} are each independently selected from C₁₋₄alkyl, and C₁₋₄alkyl substituted one to four times by fluoro,
guanidino,
—C(O)OH,
—C(O)OR^{x},
   where R^{x} is selected from C₁₋₄alkyl, and C₁₋₄alkyl substituted one to four times by fluoro,
—C(O)NH₂,
—C(O)NHR^{x},
   where R^{x} is selected from C₁₋₄alkyl, and C₁₋₄alkyl substituted one to four times by fluoro,
—C(O)NR^{x1}R^{x2},
   where R^{x1} and R^{x2} are each independently selected from C₁₋₄alkyl, and C₁₋₄alkyl substituted one to four times by fluoro,
—S(O)₂NH₂,
—NHS(O)₂H,
—NHC(O)H,
—NHC(O)NH₂,
nitro, and
cyano.
   Suitably “substituted” means the subject chemical moiety has from one to four substituents selected from the group consisting of:
fluoro,
chloro,
bromo,
iodo,
C₁₋₄alkyl,
C₁₋₄alkyl substituted with from 1 to 4 substituents independently selected from: fluoro, oxo, —OH, —COOH, —NH₂, —NHCH₃, —N(CH₃)₂, —OCH₃, —OCH₂CH₃, and —CN,
—OC₁₋₄alkyl,
—OC₁₋₄alkyl substituted with from 1 to 4 substituents independently selected from: fluoro, oxo, —OH, —COOH, —NH₂, —NHCH₃, —N(CH₃)₂, and —CN,
—SH,
—S(O)₂H,
oxo,
hydroxy,
amino,
—NHR^{x},
   where R^{x} is selected from C₁₋₄alkyl, and C₁₋₆alkyl substituted one to 4 times by fluoro,
—NR^{x1}R^{x2},
   where R^{x1} and R^{x2} are each independently selected from C₁₋₄alkyl, and C₁₋₄alkyl substituted one to four times by fluoro,
guanidino,
—C(O)OH,
—C(O)OR^{x},
   where R^{x} is selected from C₁₋₄alkyl, and C₁₋₄alkyl substituted one to four times by fluoro,
—C(O)NH₂,
—C(O)NHR^{x},
   where R^{x} is selected from C₁₋₄alkyl, and C₁₋₄alkyl substituted one to four times by fluoro,
—C(O)NR^{x1}R^{x2},
   where R^{x1} and R^{x2} are each independently selected from C₁₋₄alkyl, and C₁₋₄alkyl substituted one to four times by fluoro,
—S(O)₂NH₂,
—NHS(O)₂H,
—NHC(O)H,
—NHC(O)NH₂,
nitro, and
cyano.
   Suitably “substituted” means the subject chemical moiety has from one to three substituents selected from the group consisting of:
fluoro,
chloro,
bromo,
—OC₁₋₄alkyl,
oxo,
hydroxy,
amino,
—C(O)OH,
—C(O)NH₂,
nitro, and
cyano.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

Ac (acetyl);
ACN (acetonitrile);
BH₃.Me₂S (borane dimethylsulfide compex);
Bn (benzyl);
Boc (tert-Butoxycarbonyl);
CAN (cerric ammonium nitrate);
C18 (refers to 18-carbon alkyl groups on silicon in HPLC stationary phase);
CH₃CN (acetonitrile);
DCM (dichloromethane);
DIAD (diisopropyl azodicarboxylate);
Dioxane (1,4-dioxane);

### DMF (N,N-dimethylformamide);

DMSO (dimethylsulfoxide);
Et₃N (triethylamine);
EtOAc (ethyl acetate);
Et₂O (diethyl ether);
HCl (hydrochloric acid);
HEPES (4-(2-hydroxyethyl)-1-piperazine ethane sulfonic acid);
HPLC (high pressure liquid chromatography);
IPA (isopropyl alcohol);
K₂CO₃ (potassium carbonate);
LiOH.H₂O (lithium hydroxide monohydrate);
MeOH (methanol);
NaCNBH₃ (sodium cyanoborohydride);
NaHCO₃ (sodium bicarbonate);
NaOH (sodium hydroxide);
Na₂SO₄ (sodium sulfate);
NH₄Cl (ammonium chloride);
rt (room temperature);
TLC (thin layer chromatography);
TEA (triethylamine);
TFA (trifluoroacetic acid);
THF (tetrahydrofuran); and
T3P® (2,4,6-tripropyl-1,3,5,2,4,6-trioxatriphosphorinane-2,4,6-trioxide).
All references to ether are to diethyl ether and brine refers to a saturated aqueous solution of NaCl.

### Compound Preparation

The compounds according to Formula (X) are prepared using conventional organic synthetic methods. A suitable synthetic route is depicted below in the following general reaction schemes. All of the starting materials are commercially available or are readily prepared from commercially available starting materials by those of skill in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Organic Synthesis (4th ed.), John Wiley & Sons, NY (2006). In some instances, a substituent may be specifically selected to be reactive under the reaction conditions used. Under these circumstances, the reaction conditions convert the selected substituent into another substituent that is either useful as an intermediate compound or is a desired substituent in a target compound.

As used in the Schemes below, the specified groups, such as r and r′ represent all corresponding positional combinations on all of the Formulas disclosed herein. For example, r and r′ represent R⁵, and R⁶ of Formula (X).

### General Synthetic Schemes

The compounds of the examples decribed herein can be prepared by the synthetic route detailed in Scheme 1. Coupling of commercially-available amine i with acid ii under standard conditions (i.e. T3P, HATU) provides amide iii. Reduction of the ester functionality with either lithium aluminum hydride or lithium borohydride procures alcohol iv. Subsequent treatment with isocyanate v under basic conditions affords targeted compounds of generic structure vi.

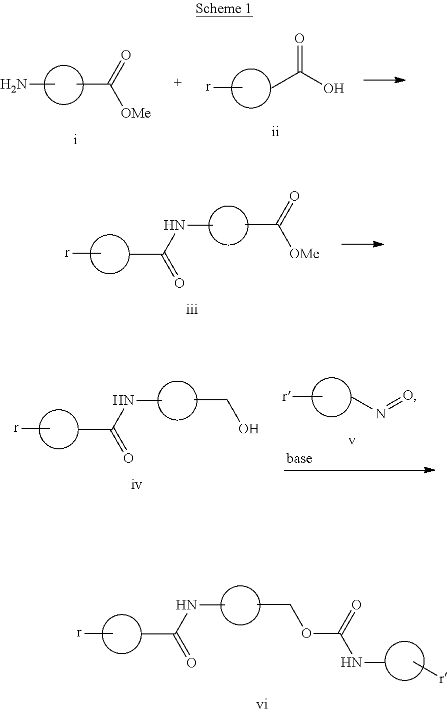

An alternative approach to synthesizing compounds of generic structure vi is described in Scheme 2. Reduction of commerically-available ester vii with lithium aluminum hydride or lithium borohydride provides alcohol viii. Subsequent treatment with isocyanate v under basic conditions affords carbamate ix. Cleavage of the Boc-carbamate under acidic conditions (i.e. HCl, TFA) procures amine x as its corresponding HCl-salt. Coupling with acid ii under standard conditions (T3P, HATU) affords targeted compounds of generic structure vi.

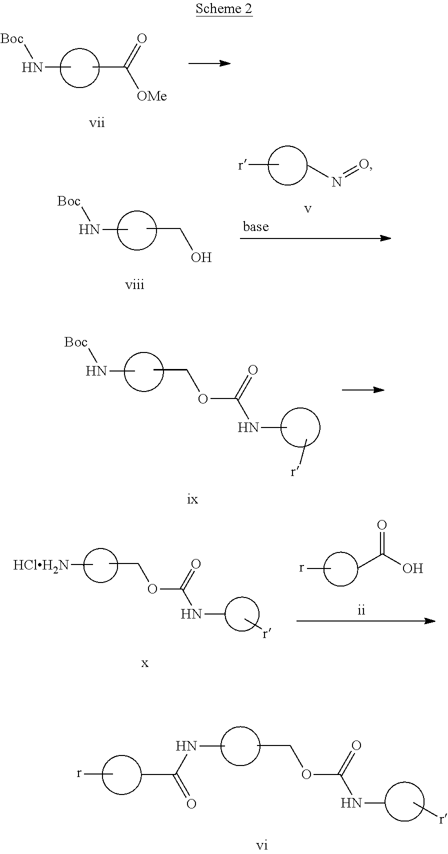

### Methods of Use

The compounds according to Formula (X) and pharmaceutically acceptable salts thereof are inhibitors of the ATF4 pathway. Compounds which are inhibitors of the ATF4 pathway are readily identified by exhibiting activity in the ATF4 Cell Based Assay below. These compounds are potentially useful in the treatment of conditions wherein the underlying pathology is attributable to (but not limited to) modulation of the eIF2α lpha pathway, for example, neurodegenerative disorders, cancer, cardiovascular and metabolic diseases. Accordingly, in another aspect the invention is directed to methods of treating such conditions.

The pharmaceutically active compounds within the scope of this invention are useful as ATF4 pathway inhibitors in mammals, particularly humans, in need thereof.

The Integrated Stress Response (ISR) is a collection of cellular stress response pathways that converge in phosphorylation of the translation initiation factor eIF2α resulting in a reduction in overall translation in cells. Mammalian cells have four eIF2α kinases that phosphorylate this initiation factor in the same residue (serine 51); PERK is activated by the accumulation of unfolded proteins in the endoplasmic reticulum (ER), GCN2 is activated by amino acid starvation, PKR by viral infection and HRI by heme deficiency. Activation of these kinases decreases bulk protein synthesis but it also culminates in increased expression of specific mRNAs that contain uORFs. Two examples of these mRNAs are the transcription factor ATF4 and the pro-apoptotic gene CHOP. Phosphorylation of eIF2α upon stress and the concomitant reduction in protein translation has been shown to both have cytoprotective and cytotoxic effects depending on the cellular context and duration and severity of the stress. An integrated stress response-associated disease is a disease characterized by increased activity in the integrated stress response (e.g. increased phosphorylation of eIF2α by an eIF2α kinase compared to a control such as a subject without the disease). A disease associated with phosphorylation of eIF2α is disease characterized by an increase in phosphorylation of eIF2α relative to a control, such as a subject without the disease.

Activation of PERK occurs upon ER stress and hypoxic conditions and its activation and effect on translation has been shown to be cytoprotective for tumor cells [17]. Adaptation to hypoxia in the tumor microenvironment is critical for survival and metastatic potential. PERK has also been shown to promote cancer proliferation by limiting oxidative DNA damage and death [18, 19]. Moreover, a newly identified PERK inhibitor has been shown to have antitumor activity in a human pancreatic tumor xenograft model [20]. Compounds disclosed herein decrease the viability of cells that are subjected to ER-stress. Thus, pharmacological and acute inhibition of the PERK branch with the compounds disclosed herein results in reduced cellular fitness. During tumor growth, compounds disclosed herein, that block the cytoprotective effects of eIF2α phosphorylation upon stress may prove to be potent anti-proliferative agents.

It is known that under certain stress conditions several eIF2α kinases can be simultaneously activated. For example, during tumor growth, the lack of nutrients and hypoxic conditions are known to both activate GCN2 and PERK. Like PERK, GCN2 and their common target, ATF4, have been proposed to play a cytoprotective role [21]. By blocking signaling by both kinases, compounds disclosed herein may bypass the ability of the ISR to protect cancer cells against the effects of low nutrients and oxygen levels encountered during the growth of the tumor.

Prolonged ER stress leads to the accumulation of CHOP, a pro-apoptotic molecule. In a prion mouse model, overexpression of the phosphatase of eIF2α increased survival of prion-infected mice whereas sustained eIF2α phosphorylation decreased survival [22]. The restoration of protein translation rates during prion disease was shown to rescue synaptic deficits and neuronal loss. The compounds disclosed herein that make cells insensitive to eIF2α phosphorylation sustain protein translation. Compounds disclosed herein could prove potent inhibitors of neuronal cell death in prion disease by blocking the deleterious effects of prolonged eIF2α phosphorylation. Given the prevalence of protein misfolding and activation on the UPR in several neurodegenerative diseases (e.g. Alzheimer's (AD) and Parkinson's (PD)), manipulation of the PERK-eIF2α branch could prevent synaptic failure and neuronal death across the spectrum of these disorders.

Another example of tissue-specific pathology that is linked to heightened eIF2α phosphorylation is the fatal brain disorder, vanishing white matter disease (VWM) or childhood ataxia with CNS hypomyelination (CACH). This disease has been linked to mutation in eIF2B, the GTP exchange factor that is necessary for eIF2 function in translation [23]. eIF2α phosphorylation inhibits the activity of eIF2B and mutations in this exchange factor that reduce its exchange activity exacerbate the effects of eIF2α phosphorylation. The severe consequences of the CACH mutations point to the dangers of UPR hyper-activation, especially as it pertains to the myelin-producing oligodendrocyte. Small molecules, such as compounds disclosed herein, that block signaling through eIF2α phosphorylation may reduce the deleterious effects of its hyper-activation in VWM.

In another aspect is provided a method of improving long-term memory in a patient, which comprises administering a therapeutically effective amount of a compound of Formula (X) to the patient. In embodiments, the patient is human. In embodiments, the patient is a mammal.

The compounds of this invention inhibit the integrated stress response which is implicated in the pathogenesis of neurological disorders. Suitably the present invention relates to a method for treating or lessening the severity of neurological disorders. Suitably, the disorders treatable with the compounds of the invention include: Alcoholism, Anxiety, Depression, Schizophrenia, Bipolar Disorder, Obsessive Compulsive Disorder, Panic Disorder, Chronic Pain, Obesity, Senile Dementia, Migraine, Bulimia, Anorexia, Social Phobia, Pre-Menstrual Syndrome (PMS), Adolescent Depression, Trichotillomania, Dysthymia and Substance Abuse.

In embodiments, the neurological disorder is treated in a human patient.

The compounds of this invention inhibit the integrated stress response which is implicated in the pathogenesis of pain. Visceral pain is pain associated with the viscera, which encompass the internal organs of the body. These organs include, e.g., the heart, lungs, reproductive organs, bladder, ureters, the digestive organs, liver, pancreas, spleen, and kidneys. There are a variety of conditions in which visceral pain may exist, such as, for example, pancreatitis, labor, abdominal surgery associated with ileus, cystitis, menstrual period, or dysmenorrhea. Likewise, kidney pain, epigastric pain, pleural pain, and painful biliary colic, appendicitis pain may all be considered to be visceral pain. Substernal pain or pressure from early myocardial infarction is also visceral. Diseases of the stomach, dudenurn or colon can cause visceral pain. Commonly encountered gastrointestinal (GI) disorders that cause visceral pain include functional bowel disorder (FBD) and inflammatory bowel disease (IBD). These GI disorders include a wide range of disease states that are currently only moderately controlled, including, with respect to FBD, gastro-esophageal reflux, dyspepsia, irritable bowel syndrome (IBS) and functional abdominal pain syndrome (FADS), and, with respect to IBD, Crohn's disease, ileitis and ulcerative colitis, all of which regularly produce visceral pain.

Suitably the present invention relates to a method for treating or lessening the severity of pain. The invention can alleviate pain from many causes, including but not limited to shock; limb amputation; severe chemical or thermal burn injury; sprains, ligament tears, fractures, wounds and other tissue injuries; dental surgery, procedures and maladies; labor and delivery; migraine; during physical therapy; post operative pain; radiation poisoning; cancer; acquired immunodeficiency syndrome (AIDS); epidural (or peridural) fibrosis; faded back surgery and faded laminectomy; sciatica; painful sickle cell crisis: arthritis; autoimmune disease; intractable bladder pain; and the like. The present invention is directed to the treatment of intractible pain, whatever its cause.

In embodiments, pain is treated in a human patient.

The compounds of this invention inhibit the unfolded protein response which is implicated in the pathogenesis of inter vertebral disc degeneration. Suitably the present invention relates to a method for treating or lessening the severity of vertebral disc degeneration.

In embodiments, the compounds set forth herein are provided as pharmaceutical compositions comprising the compound and a pharmaceutically acceptable excipient. In embodiments of the method, the compound, or a pharmaceutically acceptable salt thereof, is co-adminstered with a second agent (e.g. therapeutic agent). In embodiments of the method, the compound, or a pharmaceutically acceptable salt thereof, is co-adminstered with a second agent (e.g. therapeutic agent), which is administered in a therapeutically effective amount. In embodiments, the second agent is an agent for improving memory.

Induction of long-term memory (LTM) has been shown to be facilitated by decreased and impaired by increased eIF2α phosphorylation. The data strongly support the notion that under physiological conditions, a decrease in eIF2α phosphorylation constitutes a critical step for the long term synaptic changes required for memory formation and ATF4 has been shown to be an important regulator of these processes [24] [25] [26]. It is not known what the contributions of the different eIF2α kinases to learning is or whether each play a differential role in the different parts of the brain. Regardless of the eIF2α kinase/s responsible for phosphorylation of eIF2α in the brain, compounds disclosed herein that block translation and ATF4 production make them ideal molecules to block the effects of this phosphorylation event on memory. Pharmacological treatment with compounds disclosed herein increase spatial memory and enhance auditory and contextual fear conditioning.

Regulators of translation, such as the compounds of Formula (X), could serve as therapeutic agents that improve memory in human disorders associated with memory loss such as Alzheimer's disease and in other neurological disorders that activate the UPR in neurons and thus could have negative effects on memory consolidation such as Parkinson's disease, Amyotrophic lateral sclerosis and prion diseases. In addition, a mutation in eIF2γ, that disrupts complex integrity linked intellectual disability (intellectual disability syndrome or ID) to impaired translation initiation in humans [27]. Hence, two diseases with impaired eIF2 function, ID and VWM, display distinct phenotypes but both affect mainly the brain and impair learning.

The compounds of Formula (X) are also useful in applications where increasing protein production output is desirable, such as in vitro cell free systems for protein production. In vitro systems have basal levels of eIF2α phosphorylation that reduce translational output [28, 29]. Similarly, production of antibodies by hybridomas may also be improved by addition of compounds disclosed herein.

In another aspect is provided a method of increasing protein expression of a cell or in vitro expression system, which comprises administering an effective amount of a compound of Formula (X) to the cell or expression system. In embodiments, the method is a method of increasing protein expression by a cell and includes administering an effective amount of a compound of Formula (X) to the cell. In embodiments, the method is a method of increasing protein expression by an in vitro protein expression system and includes administering an effective amount of a compound of Formula (X) to the in vitro (e.g. cell free) protein expression system.

In embodiments, the compounds set forth herein are provided as pharmaceutical compositions comprising the compound and a pharmaceutically acceptable excipient. In embodiments of the method, the compound, or a pharmaceutically acceptable salt thereof, is co-adminstered with a second agent. In embodiments of the method, the compound, or a pharmaceutically acceptable salt thereof, is co-adminstered with a second agent, which is administered in a therapeutically effective amount. In embodiments, the second agent is an agent for improving protein expression.

Suitably, the present invention relates to a method for treating or lessening the severity of breast cancer, including inflammatory breast cancer, ductal carcinoma, and lobular carcinoma.

Suitably the present invention relates to a method for treating or lessening the severity of colon cancer.

Suitably the present invention relates to a method for treating or lessening the severity of pancreatic cancer, including insulinomas, adenocarcinoma, ductal adenocarcinoma, adenosquamous carcinoma, acinar cell carcinoma, and glucagonoma.

Suitably the present invention relates to a method for treating or lessening the severity of skin cancer, including melanoma, including metastatic melanoma.

Suitably the present invention relates to a method for treating or lessening the severity of lung cancer including small cell lung cancer, non-small cell lung cancer, squamous cell carcinoma, adenocarcinoma, and large cell carcinoma.

Suitably the present invention relates to a method for treating or lessening the severity of cancers selected from the group consisting of brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, head and neck, kidney, liver, melanoma, ovarian, pancreatic, adenocarcinoma, ductal adenocarcinoma, adenosquamous carcinoma, acinar cell carcinoma, glucagonoma, insulinoma, prostate, sarcoma, osteosarcoma, giant cell tumor of bone, thyroid, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), neuroendocrine cancers and testicular cancer.

Suitably the present invention relates to a method for treating or lessening the severity of pre-cancerous syndromes in a mammal, including a human, wherein the pre-cancerous syndrome is selected from: cervical intraepithelial neoplasia, monoclonal gammapathy of unknown significance (MGUS), myelodysplastic syndrome, aplastic anemia, cervical lesions, skin nevi (pre-melanoma), prostatic intraepithleial (intraductal) neoplasia (PIN), Ductal Carcinoma in situ (DCIS), colon polyps and severe hepatitis or cirrhosis.

Suitably the present invention relates to a method for treating or lessening the severity of neurodegenerative diseases/injury, such as Alzheimer's disease, spinal cord injury, traumatic brain injury, ischemic stroke, stroke, diabetes, Parkinson disease, Huntington's disease, Creutzfeldt-Jakob Disease, and related prion diseases, progressive supranuclear palsy, amyotrophic lateral sclerosis, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementia, cognitive impairment, atherosclerosis, ocular diseases, arrhythmias, in organ transplantation and in the transportation of organs for transplantation.

Suitably the present invention relates to a method for preventing organ damage during and after organ transplantation and in the transportation of organs for transplantation. The method of preventing organ damage during and after organ transplantation comprises the in vivo administration of a compound of Formula (X). The method of preventing organ damage during the transportation of organs for transplantation comprises adding a compound of Formula (X) to the solution housing the organ during transportation.

Suitably, the present invention relates to a method for treating or lessening the severity of neurodegernative ocular diseases, wherein the disease is retinitis pigmentosa.

Suitably, the present invention relates to a method for treating or lessening the severity of ocular diseases, wherein the disease is selected from retinal dystrophies and corneal dystrophies, such as Fuch's corneal dystrophy.

Suitably the present invention relates to a method for treating or lessening the severity of ocular diseases/angiogenesis. The method of treating or lessening the severity of ocular diseases/angiogenesis comprises the in vivo administration of a compound of Formula (X). In embodiments of methods according to the invention, the disorder of ocular diseases, including vascular leakage can be: edema or neovascularization for any occlusive or inflammatory retinal vascular disease, such as rubeosis irides, neovascular glaucoma, pterygium, vascularized glaucoma filtering blebs, conjunctival papilloma; choroidal neovascularization, such as neovascular age-related macular degeneration (AMD), myopia, prior uveitis, trauma, or idiopathic; macular edema, such as post surgical macular edema, macular edema secondary to uveitis including retinal and/or choroidal inflammation, macular edema secondary to diabetes, and macular edema secondary to retinovascular occlusive disease (i.e. branch and central retinal vein occlusion); retinal neovascularization due to diabetes, such as retinal vein occlusion, uveitis, ocular ischemic syndrome from carotid artery disease, ophthalmic or retinal artery occlusion, sickle cell retinopathy, other ischemic or occlusive neovascular retinopathies, retinopathy of prematurity, or Eale's Disease; and genetic disorders, such as VonHippel-Lindau syndrome.

In some embodiments, the neovascular age-related macular degeneration is wet age-related macular degeneration. In other embodiments, the neovascular age-related macular degeneration is dry age-related macular degeneration and the patient is characterized as being at increased risk of developing wet age-related macular degeneration.

In embodiments, the ocular disease is treated in a human patient.

The methods of treatment of the invention comprise administering an effective amount of a compound according to Formula (X) or a pharmaceutically acceptable salt, thereof to a patient in need thereof.

The invention also provides a compound according to Formula (X) or a pharmaceutically-acceptable salt thereof for use in medical therapy, and particularly in therapy for: cancer, pre-cancerous syndromes, Alzheimer's disease, spinal cord injury, traumatic brain injury, ischemic stroke, stroke, diabetes, Parkinson disease, Huntington's disease, Creutzfeldt-Jakob Disease, and related prion diseases, progressive supranuclear palsy, amyotrophic lateral sclerosis, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementia, cognitive impairment, atherosclerosis, ocular diseases, in organ transplantation and arrhythmias. The invention also provides a compound according to Formula (X) or a pharmaceutically-acceptable salt thereof for use in preventing organ damage during the transportation of organs for transplantation. Thus, in further aspect, the invention is directed to the use of a compound according to Formula (X) or a pharmaceutically acceptable salt thereof in the manfacture of a medicament for the treatment of a disorder characterized by activation of the UPR, such as cancer, pre-cancerous syndromes, Alzheimer's disease, spinal cord injury, traumatic brain injury, ischemic stroke, stroke, diabetes, Parkinson disease, Huntington's disease, Creutzfeldt-Jakob Disease, and related prion diseases, progressive supranuclear palsy, amyotrophic lateral sclerosis, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementia, cognitive impairment, atherosclerosis, ocular diseases, in organ transplantation and arrhythmias.

The methods of treatment of the invention comprise administering a safe and effective amount of a compound of Formula (X), or a pharmaceutically acceptable salt thereof to a mammal, suitably a human, in need thereof.

As used herein, “treating”, and derivatives thereof, in reference to a condition means: (1) to ameliorate the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, or (4) to slow the progression of the condition or one or more of the biological manifestations of the condition.

The term “treating” and derivatives thereof refers to therapeutic therapy. Therapeutic therapy is appropriate to alleviate symptions or to treat at early signs of disease or its progression.

Prophylactic therapy or prevention therapy is appropriate when a subject has, for example, a strong family history of neurodegenerative diseases. Prophylactic therapy is appropriate when a subject has, for example, a strong family history of cancer or is otherwise considered at high risk for developing cancer, or when a subject has been exposed to a carcinogen.

The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” in reference to a compound of Formula (X), or a pharmaceutically acceptable salt thereof, means an amount of the compound sufficient to treat the patient's condition but low enough to avoid serious side effects (at a reasonable benefit/risk ratio) within the scope of sound medical judgment. A safe and effective amount of the compound will vary with the particular route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient”, and derivatives thereof refers to a human or other mammal, suitably a human.

The compounds of Formula (X) or pharmaceutically acceptable salts thereof may be administered by any suitable route of administration, including systemic administration. Systemic administration includes oral administration, and parenteral administration. Parenteral administration refers to routes of administration other than enteral, transdermal, or by inhalation, and is typically by injection or infusion. Parenteral administration includes intravenous, intramuscular, and subcutaneous injection or infusion.

The compounds of Formula (X) or pharmaceutically acceptable salts thereof may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Additionally, the compounds of Formula (X) or pharmaceutically-acceptable salts thereof may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of the compound in vivo; (b) modify the duration of action of the compound in vivo; (c) modify the transportation or distribution of the compound in vivo; (d) modify the solubility of the compound in vivo; and (e) overcome a side effect or other difficulty encountered with the compound. Where a —COOH or —OH group is present, pharmaceutically acceptable esters can be employed, for example methyl, ethyl, and the like for —COOH, and acetate, maleate, and the like for —OH, and those esters known in the art for modifying solubility or hydrolysis characteristics.

The compounds of Formula (X) and pharmaceutically acceptable salts thereof may be co-administered with at least one other active agent known to be useful in the treatment of cancer or pre-cancerous syndromes.

By the term “co-administration” as used herein is meant either simultaneous administration or any manner of separate sequential administration of an ATF4 pathway inhibiting compound, as described herein, and a further active agent or agents, known to be useful in the treatment of cancer, including chemotherapy and radiation treatment. The term further active agent or agents, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered by injection and another compound may be administered orally.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita and S. Hellman (editors), 6th edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; cell cycle signaling inhibitors; proteasome inhibitors; and inhibitors of cancer metabolism.

Examples of a further active ingredient or ingredients (anti-neoplastic agent) for use in combination or co-administered with the presently invented ATF4 pathway inhibiting compounds are chemotherapeutic agents.

Suitably, the pharmaceutically active compounds of the invention are used in combination with a VEGFR inhibitor, suitably 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt thereof, which is disclosed and claimed in in International Application No. PCT/US01/49367, having an International filing date of Dec. 19, 2001, International Publication Number WO02/059110 and an International Publication date of Aug. 1, 2002, the entire disclosure of which is hereby incorporated by reference, and which is the compound of Example 69. 5-[[4-[(2,3-dimethyl-2H-indazol-6-yl)methylamino]-2-pyrimidinyl]amino]-2-methylbenzenesulfonamide can be prepared as described in International Application No. PCT/US01/49367.

In one embodiment, the cancer treatment method of the claimed invention includes the co-administration a compound of Formula (I) and/or a pharmaceutically acceptable salt thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, cell cycle signaling inhibitors; proteasome inhibitors; and inhibitors of cancer metabolism.

“Chemotherapeutic” or “chemotherapeutic agent” is used in accordance with its plain ordinary meaning and refers to a chemical composition or compound having antineoplastic properties or the ability to inhibit the growth or proliferation of cells.

Additionally, the compounds described herein can be co-administered with conventional immunotherapeutic agents including, but not limited to, immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to¹¹¹In, ⁹⁰Y, or¹³¹I, etc.).

In a further embodiment, the compounds described herein can be co-administered with conventional radiotherapeutic agents including, but not limited to, radionuclides such as ⁴⁷Sc, ⁶⁴C ⁶⁷C, ⁸⁹Sr, ⁸⁶Y, ⁸⁷Y, and ²¹²Bi, optionally conjugated to antibodies directed against tumor antigens.

Additional examples of a further active ingredient or ingredients (anti-neoplastic agent) for use in combination or co-administered with the presently invented ATF4 pathway inhibiting compounds are anti-PD-L1 agents.

Anti-PD-L1 antibodies and methods of making the same are known in the art.

Such antibodies to PD-L1 may be polyclonal or monoclonal, and/or recombinant, and/or humanized.

Exemplary PD-L1 antibodies are disclosed in:
U.S. Pat. No. 8,217,149; Ser. No. 12/633,339;
U.S. Pat. No. 8,383,796; Ser. No. 13/091,936;
U.S. Pat. No. 8,552,154; Ser. No. 13/120,406;
US patent publication No. 20110280877; Ser. No. 13/068,337;
US Patent Publication No. 20130309250; Ser. No. 13/892,671;
WO2013019906;
WO2013079174;
U.S. application Ser. No. 13/511,538 (filed Aug. 7, 2012), which is the US National Phase of International Application No. PCT/US10/58007 (filed 2010);
and
U.S. application Ser. No. 13/478,511 (filed May 23, 2012).

Additional exemplary antibodies to PD-L1 (also referred to as CD274 or B7-H1) and methods for use are disclosed in U.S. Pat. No. 7,943,743; US20130034559, WO2014055897, U.S. Pat. Nos. 8,168,179; and 7,595,048. PD-L1 antibodies are in development as immuno-modulatory agents for the treatment of cancer.

In one embodiment, the antibody to PD-L1 is an antibody disclosed in U.S. Pat. No. 8,217,149. In another embodiment, the anti-PD-L1 antibody comprises the CDRs of an antibody disclosed in U.S. Pat. No. 8,217,149.

In another embodiment, the antibody to PD-L1 is an antibody disclosed in U.S. application Ser. No. 13/511,538. In another embodiment, the anti-PD-L1 antibody comprises the CDRs of an antibody disclosed in U.S. application Ser. No. 13/511,538.

In another embodiment, the antibody to PD-L1 is an antibody disclosed in application Ser. No. 13/478,511. In another embodiment, the anti-PD-L1 antibody comprises the CDRs of an antibody disclosed in U.S. application Ser. No. 13/478,511.

In one embodiment, the anti-PD-L1 antibody is BMS-936559 (MDX-1105). In another embodiment, the anti-PD-L1 antibody is MPDL3280A (RG7446). In another embodiment, the anti-PD-L1 antibody is MED14736. In another embodiment, the anti-PD-L1 antibody is atezolizumab. In another embodiment, the anti-PD-L1 antibody is avelumab. In another embodiment, the anti-PD-L1 antibody is durvalumab.

Additional examples of a further active ingredient or ingredients (anti-neoplastic agent) for use in combination or co-administered with the presently invented ATF4 pathway inhibiting compounds are PD-1 antagonist.

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any embodiments of the aspects or embodiments of the present invention in which a human individual is to be treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists useful in the any of the aspects of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

Examples of mAbs that bind to human PD-1, and useful in the various aspects and embodiments of the present invention, are described in U.S. Pat. Nos. 7,488,802, 7,521,051, 8,008,449, 8,354,509, 8,168,757, WO2004/004771, WO2004/072286, WO2004/056875, and US2011/0271358.

Specific anti-human PD-1 mAbs useful as the PD-1 antagonist in any of the aspects and embodiments of the present invention include: MK-3475, a humanized IgG4 mAb with the structure described in WHO Drug Information, Vol. 27, No. 2, pages 161-162 (2013) and which comprises the heavy and light chain amino acid sequences shown in FIG. 6; nivolumab, a human IgG4 mAb with the structure described in WHO Drug Information, Vol. 27, No. 1, pages 68-69 (2013) and which comprises the heavy and light chain amino acid sequences shown in FIG. 7; the humanized antibodies h409A11, h409A16 and h409A17, which are described in WO2008/156712, and AMP-514, which is being developed by Medimmune.

Other PD-1 antagonists useful in the any of the aspects and embodiments of the present invention include an immunoadhesin that specifically binds to PD-1, and preferably specifically binds to human PD-1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

Other examples of mAbs that bind to human PD-L1, and useful in the treatment method, medicaments and uses of the present invention, are described in WO2013/019906, WO2010/077634 A1 and U.S. Pat. No. 8,383,796. Specific anti-human PD-L1 mAbs useful as the PD-1 antagonist in the treatment method, medicaments and uses of the present invention include MPDL3280A, BMS-936559, MED14736, MSB0010718C.

KEYTRUDA®/pembrolizumab is an anti-PD-1 antibody marketed for the treatment of lung cancer by Merck. The amino acid sequence of pembrolizumab and methods of using are disclosed in U.S. Pat. No. 8,168,757.

Opdive/nivolumab is a fully human monoclonal antibody marketed by Bristol Myers Squibb directed against the negative immunoregulatory human cell surface receptor PD-1 (programmed death-1 or programmed cell death-1/PCD-1) with immunopotentiation activity. Nivolumab binds to and blocks the activation of PD-1, an Ig superfamily transmembrane protein, by its ligands PD-L1 and PD-L2, resulting in the activation of T-cells and cell-mediated immune responses against tumor cells or pathogens. Activated PD-1 negatively regulates T-cell activation and effector function through the suppression of P13k/Akt pathway activation. Other names for nivolumab include: BMS-936558, MDX-1106, and ONO-4538. The amino acid sequence for nivolumab and methods of using and making are disclosed in U.S. Pat. No. 8,008,449.

Additional examples of a further active ingredient or ingredients (anti-neoplastic agent) for use in combination or co-administered with the presently invented ATF4 pathway inhibiting compounds are immuno-modulators.

As used herein “immuno-modulators” refer to any substance including monoclonal antibodies that affects the immune system. The ICOS binding proteins of the present invention can be considered immune-modulators. Immuno-modulators can be used as anti-neoplastic agents for the treatment of cancer. For example, immune-modulators include, but are not limited to, anti-CTLA-4 antibodies such as ipilimumab (YERVOY®) and anti-PD-1 antibodies (Opdive/nivolumab and Keytrude/pembrolizumab). Other immuno-modulators include, but are not limited to, OX-40 antibodies, PD-L1 antibodies, LAG3 antibodies, TIM-3 antibodies, 41 BB antibodies and GITR antibodies.

Yervoy® (ipilimumab) is a fully human CTLA-4 antibody marketed by Bristol Myers Squibb. The protein structure of ipilimumab and methods are using are described in U.S. Pat. Nos. 6,984,720 and 7,605,238.

Suitably, the compounds of the invention are combined with an inhibitor of the activity of the protein kinase R (PKR)-like ER kinase, PERK.

Suitably, the compounds of the invention are combined with an inhibitor of the activity of the eIF2α kinases protein kinase R, (PKR), Heme-regulated eIF2α kinase (HRI), or general control non-derepressible 2 (GCN2).

Suitably, the compounds of Formula (X) and pharmaceutically acceptable salts thereof may be co-administered with at least one other active agent known to be useful in the treatment of neurodegenerative diseases/injury.

Suitably, the compounds of Formula (X) and pharmaceutically acceptable salts thereof may be co-administered with at least one other active agent known to be useful in the treatment of diabetes.

Suitably, the compounds of Formula (X) and pharmaceutically acceptable salts thereof may be co-administered with at least one other active agent known to be useful in the treatment of cardiovascular disease.

Suitably, the compounds of Formula (X) and pharmaceutically acceptable salts thereof may be co-administered with at least one other active agent known to be useful in the treatment of ocular diseases.

The compounds described herein can be used in combination with one another, with other active agents known to be useful in treating cancer (e.g. pancreatic cancer, breast cancer, multiple myeloma, or cancers of secretory cells), neurodegenerative diseases, vanishing white matter disease, childhood ataxia with CNS hypomyelination, and/or intellectual disability syndromes (e.g. associated with impaired function of eIF2 or components in a signal transduction pathway including eIF2), or with adjunctive agents that may not be effective alone, but may contribute to the efficacy of the active agent.

In embodiments, the compounds set forth herein are provided as pharmaceutical compositions comprising the compound and a pharmaceutically acceptable excipient. In embodiments of the method, the compound, or a pharmaceutically acceptable salt thereof, is co-adminstered with a second agent (e.g. therapeutic agent). In embodiments of the method, the compound, or a pharmaceutically acceptable salt thereof, is co-adminstered with a second agent (e.g. therapeutic agent), which is administered in a therapeutically effective amount. In embodiments of the method, the second agent is an agent for treating cancer (e.g. pancreatic cancer, breast cancer, multiple myeloma, or cancers of secretory cells), neurodegenerative diseases, vanishing white matter disease, childhood ataxia with CNS hypomyelination, and/or intellectual disability syndromes (e.g. associated with impaired function of eIF2 or components in a signal transduction pathway including eIF2), or an inflammatory disease (e.g. POCD or TBI). In embodiments, the second agent is an anti-cancer agent. In embodiments, the second agent is a chemotherapeutic. In embodiments, the second agent is an agent for improving memory. In embodiments, the second agent is an agent for treating a neurodegenerative disease. In embodiments, the second agent is an agent for treating vanishing white matter disease. In embodiments, the second agent is an agent for treating childhood ataxia with CNS hypo-myelination. In embodiments, the second agent is an agent for treating an intellectual disability syndrome. In embodiments, the second agent is an agent for treating pancreatic cancer. In embodiments, the second agent is an agent for treating breast cancer. In embodiments, the second agent is an agent for treating multiple myeloma. In embodiments, the second agent is an agent for treating myeloma. In embodiments, the second agent is an agent for treating a cancer of a secretory cell. In embodiments, the second agent is an agent for reducing eIF2a phosphorylation. In embodiments, the second agent is an agent for inhibiting a pathway activated by eIF2α phosphorylation. In embodiments, the second agent is an agent for inhibiting the integrated stress response. In embodiments, the second agent is an anti-inflammatory agent.

The term “eIF2α lpha” or “eIF2a” refers to the protein “Eukaryotic translation initiation factor 2A”. In embodiments, “eIF2α lpha” or “eIF2a” refers to the human protein. Included in the term “eIF2α lpha” or “eIF2a” are the wildtype and mutant forms of the protein. In embodiments, “eIF2α lpha” or “eIF2a” refers to the protein associated with Entrez Gene 83939, OMIM 609234, UniProt Q9BY44, and/or RefSeq (protein) NP 114414.

Suitably, the present invention relates to a method for treating an integrated stress response associated disease in a patient in need of such treatment, the method including administering a therapeutically effective amount of a compound of Formula (I), or a pharmaceutically acceptable salt thereof, to the patient.

Suitably, the integrated stress response-associated disease is cancer. Suitably, the integrated stress response-associated disease is a neurodegenerative disease. Suitably, the integrated stress response-associated disease is vanishing white matter disease. Suitably, the integrated stress response-associated disease is childhood ataxia with CNS hypomyelination. Suitably, the integrated stress response-associated disease is an intellectual disability syndrome.

Suitably, the present invention relates to a method for treating a disease associated with phosphorylation of eIF2α in a patient in need of such treatment, which comprises administering a therapeutically effective amount of a compound of Formula (I), or a pharmaceutically acceptable salt thereof, to the patient.

Suitably, the disease associated with phosphorylation of eIF2 α is cancer. Suitably, the disease associated with phosphorylation of eIF2 α is a neurodegenerative disease. Suitably, the disease associated with phosphorylation of eIF2 α is vanishing white matter disease. Suitably, the disease associated with phosphorylation of eIF2 α is childhood ataxia with CNS hypomyelination. Suitably, the disease associated with phosphorylation of eIF2 α is an intellectual disability syndrome.

Suitably, the present invention relates to a method for treating a disease selected from the group consisting of cancer, a neurodegenerative disease, vanishing white matter disease, childhood ataxia with CNS hypomyelination, and an intellectual disability syndrome.

Suitably, the present invention relates to a method for treating an inflammatory disease in a patient in need of such treatment, which comprises administering a therapeutically effective amount of a compound of Formula (I), or a pharmaceutically acceptable salt thereof, to the patient.

Suitably, the inflammatory disease is associated with neurological inflammation. Suitably, the inflammatory disease is postoperative cognitive dysfunction. Suitably, the inflammatory disease is traumatic brain injury or chronic traumatic encephalopathy (CTE).

In embodiments of the method of treating a disease, the disease is selected from the group consisting of cancer, a neurodegenerative disease, vanishing white matter disease, childhood ataxia with CNS hypomyelination, and an intellectual disability syndrome. In embodiments of the method of treating a disease, the disease is cancer. In embodiments of the method of treating a disease, the disease is a neurodegenerative disease. In embodiments of the method of treating a disease, the disease is vanishing white matter disease. In embodiments of the method of treating a disease, the disease is childhood ataxia with CNS hypomyelination. In embodiments of the method of treating a disease, the disease is an intellectual disability syndrome. In embodiments of the method of treating a disease, the disease is associated with phosphorylation of eIF2α. In embodiments of the method of treating a disease, the disease is associated with an eIF2α signaling pathway. In embodiments of the method of treating a disease, the disease is a cancer of a secretory cell type. In embodiments of the method of treating a disease, the disease is pancreatic cancer. In embodiments of the method of treating a disease, the disease is breast cancer. In embodiments of the method of treating a disease, the disease is multiple myeloma. In embodiments of the method of treating a disease, the disease is lymphoma. In embodiments of the method of treating a disease, the disease is leukemia. In embodiments of the method of treating a disease, the disease is a hematopoietic cell cancer.

In embodiments of the method of treating a disease, the disease is Alzheimer's disease. In embodiments of the method of treating a disease, the disease is Amyotrophic lateral sclerosis. In embodiments of the method of treating a disease, the disease is Creutzfeldt-Jakob disease. In embodiments of the method of treating a disease, the disease is frontotemporal dementia. In embodiments of the method of treating a disease, the disease is Gerstmann-Straussler-Scheinker syndrome. In embodiments of the method of treating a disease, the disease is Huntington's disease. In embodiments of the method of treating a disease, the disease is HIV-associated dementia. In embodiments of the method of treating a disease, the disease is kuru. In embodiments of the method of treating a disease, the disease is Lewy body dementia. In embodiments of the method of treating a disease, the disease is Multiple sclerosis. In embodiments of the method of treating a disease, the disease is Parkinson's disease. In embodiments of the method of treating a disease, the disease is a Prion disease.

In embodiments of the method of treating a disease, the disease is an inflammatory disease. In embodiments, the inflammatory disease is postoperative cognitive dysfunction. In embodiments, the inflammatory disease is traumatic brain injury. In embodiments, the inflammatory disease is arthritis. In embodiments, the inflammatory disease is rheumatoid arthritis. In embodiments, the inflammatory disease is psoriatic arthritis. In embodiments, the inflammatory disease is juvenile idiopathic arthritis. In embodiments, the inflammatory disease is multiple sclerosis. In embodiments, the inflammatory disease is systemic lupus erythematosus (SLE). In embodiments, the inflammatory disease is myasthenia gravis. In embodiments, the inflammatory disease is juvenile onset diabetes. In embodiments, the inflammatory disease is diabetes mellitus type 1. In embodiments, the inflammatory disease is Guillain-Barre syndrome. In embodiments, the inflammatory disease is Hashimoto's encephalitis. In embodiments, the inflammatory disease is Hashimoto's thyroiditis. In embodiments, the inflammatory disease is ankylosing spondylitis. In embodiments, the inflammatory disease is psoriasis. In embodiments, the inflammatory disease is Sjogren's syndrome. In embodiments, the inflammatory disease is vasculitis. In embodiments, the inflammatory disease is glomerulonephritis. In embodiments, the inflammatory disease is auto-immune thyroiditis. In embodiments, the inflammatory disease is Behcet's disease. In embodiments, the inflammatory disease is Crohn's disease. In embodiments, the inflammatory disease is ulcerative colitis. In embodiments, the inflammatory disease is bullous pemphigoid. In embodiments, the inflammatory disease is sarcoidosis. In embodiments, the inflammatory disease is ichthyosis. In embodiments, the inflammatory disease is Graves ophthalmopathy. In embodiments, the inflammatory disease is inflammatory bowel disease. In embodiments, the inflammatory disease is Addison's disease. In embodiments, the inflammatory disease is Vitiligo. In embodiments, the inflammatory disease is asthma. In embodiments, the inflammatory disease is allergic asthma. In embodiments, the inflammatory disease is acne vulgaris. In embodiments, the inflammatory disease is celiac disease. In embodiments, the inflammatory disease is chronic prostatitis. In embodiments, the inflammatory disease is inflammatory bowel disease. In embodiments, the inflammatory disease is pelvic inflammatory disease. In embodiments, the inflammatory disease is reperfusion injury. In embodiments, the inflammatory disease is sarcoidosis. In embodiments, the inflammatory disease is transplant rejection. In embodiments, the inflammatory disease is interstitial cystitis. In embodiments, the inflammatory disease is atherosclerosis. In embodiments, the inflammatory disease is atopic dermatitis.

In embodiments, the method of treatment is a method of prevention. For example, a method of treating postsurgical cognitive dysfunction may include preventing postsurgical cognitive dysfunction or a symptom of postsurgical cognitive dysfunction or reducing the severity of a symptom of postsurgical cognitive dysfunction by administering a compound described herein prior to surgery.

In an embodiment, this invention provides a compound of Formula (X), or a pharmaceutically acceptable salt thereof, for use in the treatment of a disease selected from the group consisting of cancer, a neurodegenerative disease, vanishing white matter disease, childhood ataxia with CNS hypomyelination, and an intellectual disability syndrome.

In an embodiment, this invention provides a compound of Formula (X), or a pharmaceutically acceptable salt thereof, for use in the treatment of an integrated stress response associated disease.

In an embodiment, this invention provides a compound of Formula (X), or a pharmaceutically acceptable salt thereof, for use in the treatment of a disease associated with phosphorylation of eIF2α.

In an embodiment, this invention provides for the use of a compound of Formula (X), or a pharmaceutically acceptable salt thereof, in the manufacture of a medicament for the treatment of a disease selected from the group consisting of cancer, a neurodegenerative disease, vanishing white matter disease, childhood ataxia with CNS hypomyelination, and an intellectual disability syndrome.

In an embodiment, this invention provides for the use of a compound of Formula (X), or a pharmaceutically acceptable salt thereof, in the manufacture of a medicament for the treatment an integrated stress response associated disease.

In an embodiment, this invention provides for the use of a compound of Formula (X), or a pharmaceutically acceptable salt thereof, in the manufacture of a medicament for the treatment of a disease associated with phosphorylation of eIF2α.

The present invention therefore provides a method of treating cancer, neurodegeneration and other conditions requiring ATF4 pathway inhibition, which comprises administering an effective amount of a compound of Formula (X) or a pharmaceutically acceptable salt thereof. The compounds of Formula (X) also provide for a method of treating the above indicated disease states because of their demonstrated ability to act as ATF4 pathway inhibitors. The drug may be administered to a patient in need thereof by any conventional route of administration, including, but not limited to, intravenous, intramuscular, oral, topical, subcutaneous, intradermal, intraocular and parenteral. Suitably, an ATF4 pathway inhibitor may be delivered directly to the brain by intrathecal or intraventricular route, or implanted at an appropriate anatomical location within a device or pump that continuously releases the ATF4 pathway inhibiting drug.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical compositions are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-100 mg/kg of active compound, preferably 0.001-50 mg/kg. When treating a human patient in need of a ATF4 pathway inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages, is preferred. Parenteral administration, at high dosages, however, also can be used when safe and convenient for the patient.

Optimal dosages to be administered may be readily determined by those skilled in the art, and will vary with the particular ATF4 pathway inhibitor in use, the strength of the preparation, the mode of administration, and the advancement of the disease condition. Additional factors depending on the particular patient being treated will result in a need to adjust dosages, including patient age, weight, diet, and time of administration.

The method of this invention of inducing ATF4 pathway inhibitory activity in mammals, including humans, comprises administering to a subject in need of such activity an effective ATF4 pathway inhibiting amount of a pharmaceutically active compound of the present invention.

The invention also provides for the use of a compound of Formula (I) or a pharmaceutically acceptable salt thereof in the manufacture of a medicament for inhibiting the ATF4 pathway.

The invention also provides for the use of a compound of Formula (X) or a pharmaceutically acceptable salt thereof in the manufacture of a medicament for treating cancer, pre-cancerous syndromes, Alzheimer's disease, spinal cord injury, traumatic brain injury, ischemic stroke, stroke, diabetes, Parkinson disease, Huntington's disease, Creutzfeldt-Jakob Disease, and related prion diseases, progressive supranuclear palsy, amyotrophic lateral sclerosis, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementia, cognitive impairment, atherosclerosis, ocular diseases, arrhythmias, in organ transplantation and in the transportation of organs for transplantation.

The invention also provides for the use of a compound of Formula (X) or a pharmaceutically acceptable salt thereof in the manufacture of a medicament for preventing organ damage during the transportation of organs for transplantation.

The invention also provides for a pharmaceutical composition for use as a ATF4 pathway inhibitor which comprises a compound of Formula (X) or a pharmaceutically acceptable salt thereof and a pharmaceutically acceptable carrier.

The invention also provides for a pharmaceutical composition for use in the treatment of cancer which comprises a compound of Formula (X) or a pharmaceutically acceptable salt thereof and a pharmaceutically acceptable carrier.

In addition, the pharmaceutically active compounds of the present invention can be co-administered with further active ingredients, such as other compounds known to treat cancer, or compounds known to have utility when used in combination with a ATF4 pathway inhibitor.

The invention also provides novel processes and novel intermediates useful in preparing the presently invented compounds.

The invention also provides a pharmaceutical composition comprising from 0.5 to 1,000 mg of a compound of Formula (X) or pharmaceutically acceptable salt thereof and from 0.5 to 1,000 mg of a pharmaceutically acceptable excipient.

Without further elaboration, it is believed that one skilled in the art can, using the preceding description, utilize the present invention to its fullest extent. The following Examples are, therefore, to be construed as merely illustrative and not a limitation of the scope of the present invention in any way.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

### Example 1

### (3-(2-(4-Chlorophenoxy)acetamido)bicyclo[1.1.1]pentan-1-yl)methyl (4-chlorophenyl)carbamate

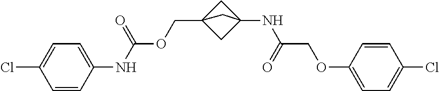 [SMILES: O=C(COc1ccc(Cl)cc1)NC12CC(COC(=O)Nc3ccc(Cl)cc3)(C1)C2]

### Step 1: Methyl 3-(2-(4-chlorophenoxy)acetamido)bicyclo[1.1.1]pentane-1-carboxylate

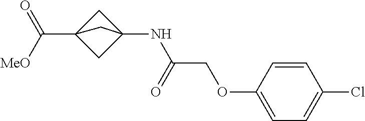 [SMILES: COC(=O)C12CC(NC(=O)COc3ccc(Cl)cc3)(C1)C2]

To a solution of commercially-available methyl 3-aminobicyclo[1.1.1]pentane-1-carboxylate, hydrochloride (0.6 g, 3.9 mmol) in dichloromethane (DCM) (15 mL) was added 2-(4-chlorophenoxy)acetic acid (0.63 g, 3.38 mmol) and TEA (1.4 mL, 10.1 mmol) followed by T3P (3.2 g, 5.1 mmol). The resulting reaction mixture was stirred at RT for 16 h. Isolute® was added to the reaction mixture, which was directly purified by silica gel chromatography (24 g column, eluting with 0-100% EtOAc:hexane) to give the title compound as a foam. LCMS m/z 310.1 (M+H)⁺.

### Step 2: 2-(4-Chlorophenoxy)-N-(3-(hydroxymethyl)bicyclo[1.1.1]pentan-1-yl)acetamide

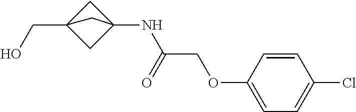 [SMILES: O=C(COc1ccc(Cl)cc1)NC12CC(CO)(C1)C2]

To a solution of methyl 3-(2-(4-chlorophenoxy)acetamido)bicyclo[1.1.1]pentane-1-carboxylate (0.9 g, 2.9 mmol) in tetrahydrofuran (THF) (25 mL) at 0° C. was added LiAlH₄ (2.9 mL, 5.8 mmol, 2M in THF) and the resulting reaction mixture was stirred at RT for 2 h. The mixture was subsequently quenched with 1 mL of water and 2 mL of sodium hydroxide (1 M, aqueous). The resulting mixture was filtered and the filtrate was concentrated under reduced pressure. The resulting residue was purified by silica gel chromatography (24 g column, 0-10% MeOH:DCM) to give the title compound as a pale yellow solid (0.7 g, 2.1 mmol, 73% yield). LCMS m/z 282.1 (M+H)⁺.

### Step 3: (3-(2-(4-Chlorophenoxy)acetamido)bicyclo[1.1.1]pentan-1-yl)methyl (4-chlorophenyl)carbamate

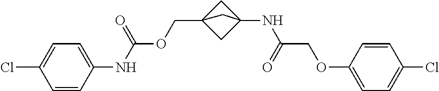 [SMILES: O=C(COc1ccc(Cl)cc1)NC12CC(COC(=O)Nc3ccc(Cl)cc3)(C1)C2]

To a solution of 2-(4-chlorophenoxy)-N-(3-(hydroxymethyl)bicyclo[1.1.1]pentan-1-yl)acetamide (100 mg, 0.36 mmol) in dichloromethane (DCM) (5 mL) was added 1-chloro-4-isocyanatobenzene (54.5 mg, 0.36 mmol) and TEA (0.099 mL, 0.710 mmol). The resulting reaction mixture was stirred at RT for 3 h, after which the crude title compound precipitated out of solution and the solvent was removed under reduced pressure. Purification by mass-guided, reverse-phase HPLC (XSELECT CSH C18 column (150 mm×30 mm i.d. 5 μm packing diameter), 15-85% H₂O (0.1% TFA):CH₃CN (0.1% TFA)) gave the title compound as a white solid. LCMS m/z 435.2 (M+H)⁺. ¹H NMR (400 MHz, DMSO-d₆) δ: 2.00 (s, 6H) 4.21 (s, 2H) 4.43 (s, 2H) 6.92-7.04 (m, 2H) 7.29-7.39 (m, 4H) 7.50 (d, J=8.62 Hz, 2H) 8.69 (s, 1H) 9.83 (br. s., 1H).

### Example 2

### 4-Chlorophenyl ((3-(2-(4-chlorophenoxy)acetamido)bicyclo[1.1.1]pentan-1-yl)methyl)carbamate

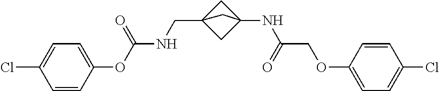 [SMILES: O=C(COc1ccc(Cl)cc1)NC12CC(CNC(=O)Oc3ccc(Cl)cc3)(C1)C2]

### Step 1: N-(3-(Aminomethyl)bicyclo[1.1.1]pentan-1-yl)-2-(4-chlorophenoxy)acetamide

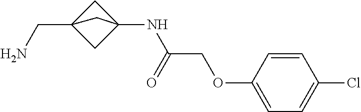 [SMILES: NCC12CC(NC(=O)COc3ccc(Cl)cc3)(C1)C2]

To a solution of 2-(4-chlorophenoxy)-N-(3-(hydroxymethyl)bicyclo[1.1.1]pentan-1-yl)acetamide (0.4 g, 1.4 mmol) in tetrahydrofuran (THF) (15 mL) was added isoindoline-1,3-dione (0.23 g, 1.6 mmol), tri-n-butylphosphine (0.52 mL, 2.1 mmol) and DEAD (0.8 mL, 2.1 mmol). The resulting reaction mixture was stirred at RT for 3 h. Following this duration, the solvent was removed under reduced pressure and the resulting residue was purified by silica gel chromatography (40 g column, 0-100% EtOAc:heptane) to give crude 2-(4-chlorophenoxy)-N-(3-((1,3-dioxoisoindolin-2-yl)methyl)bicyclo[1.1.1]pentan-1-yl)acetamide. The crude material was subsequently dissolved in ethanol (15 mL) and treated with hydrazine hydrate (0.35 mL, 7.10 mmol) at RT. The resulting reaction mixture was stirred at RT for 10 h. Following this duration, it was filtered and concentrated under reduced pressure. Purification by silica gel chromatography (12 g column, 0-20% (1% ammonium hydroxide in methanol):DCM) gave the title compound (281 mg, 1.0 mmol, 71% yield). LCMS m/z 281.1 (M+H)⁺.

### Step 2: 4-Chlorophenyl ((3-(2-(4-chlorophenoxy)acetamido)bicyclo[1.1.1]pentan-1-yl)methyl)carbamate

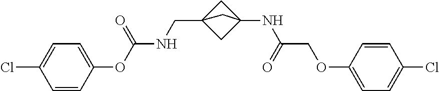 [SMILES: O=C(COc1ccc(Cl)cc1)NC12CC(CNC(=O)Oc3ccc(Cl)cc3)(C1)C2]

To a solution of N-(3-(aminomethyl)bicyclo[1.1.1]pentan-1-yl)-2-(4-chlorophenoxy)acetamide (100 mg, 0.36 mmol) in tetrahydrofuran (THF) (2 mL) was added 4-chlorophenyl carbonochloridate (0.05 mL, 0.36 mmol) at RT followed by DIPEA (0.124 mL, 0.712 mmol). The resulting reaction mixture was stirred at RT for 20 min, after which the solvent was removed under reduced pressure. Purification by mass-guided, reverse-phase HPLC (XSELECT CSH C18 column (150 mm×30 mm i.d. 5 μm packing diameter), 15-85% H₂O (0.1% TFA):CH₃CN (0.1% TFA)) gave the title compound (137 mg, 0.30 mmol, 84% yield). LCMS m/z 435.1 (M+H)⁺. ¹H NMR (400 MHz, DMSO-d₆) δ: 1.94 (s, 6H) 3.24 (d, J=5.83 Hz, 2H) 4.43 (s, 2H) 6.91-7.03 (m, 2H) 7.10-7.19 (m, 2H) 7.32-7.38 (m, 2H) 7.41-7.49 (m, 2H) 7.90 (t, J=5.83 Hz, 1H) 8.63 (s, 1H).

### Example 3

### (3-(2-(4-chloro-3-fluorophenoxy)acetamido)bicyclo[1.1.1]pentan-1-yl)methyl (4-chlorophenyl)carbamate

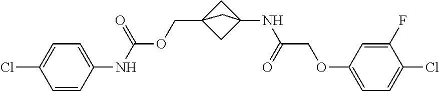 [SMILES: O=C(COc1ccc(Cl)c(F)c1)NC12CC(COC(=O)Nc3ccc(Cl)cc3)(C1)C2]

### Step 1: (3-(2-(4-Chloro-3-fluorophenoxy)acetamido)bicyclo[1.1.1]pentan-1-yl)methyl (4-chlorophenyl)carbamate

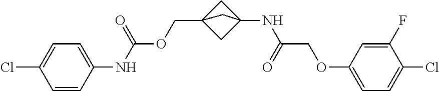 [SMILES: O=C(COc1ccc(Cl)c(F)c1)NC12CC(COC(=O)Nc3ccc(Cl)cc3)(C1)C2]

Prepared analogously to Example 1, using 2-(4-chloro-3-fluorophenoxy)acetic acid. LCMS m/z 452.1 (M+H)⁺. ¹H NMR (400 MHz, DMSO-d₆) δ ppm 2.00 (s, 6H) 4.21 (s, 2H) 4.43 (s, 2H) 6.85 (m, 1H) 7.04 (m, 1H) 7.39-7.5 (m, 5H) 8.69 (s, 1H) 9.83 (br. s., 1H).

### Example 4

### 2-(4-Chlorophenoxy)-N-(3-((3-(4-chlorophenyl)ureido)methyl)bicyclo[1.1.1]pentan-1-yl)acetamide

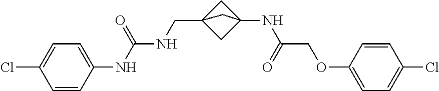 [SMILES: O=C(COc1ccc(Cl)cc1)NC12CC(CNC(=O)Nc3ccc(Cl)cc3)(C1)C2]

### Step 1: 2-(4-Chlorophenoxy)-N-(3-((3-(4-chlorophenyl)ureido)methyl)bicyclo[1.1.1]pentan-1-yl)acetamide

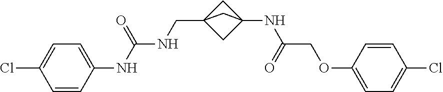 [SMILES: O=C(COc1ccc(Cl)cc1)NC12CC(CNC(=O)Nc3ccc(Cl)cc3)(C1)C2]

To the solution of N-(3-(aminomethyl)bicyclo[1.1.1]pentan-1-yl)-2-(4-chlorophenoxy)acetamide (53 mg, 0.19 mmol) in dichloromethane (DCM) (3 mL) was added 1-chloro-4-isocyanatobenzene (29.0 mg, 0.19 mmol) and TEA (0.05 mL, 0.38 mmol). The resulting reaction mixture was stirred at RT for 3 h. The crude product precipitated out of solution and the solvent was removed under reduced pressure. Trituration with diethyl ether and hexane afforded the title compound (50 mg, 0.11 mmol, 60% yield). LCMS m/z 434.1 (M+H)⁺. ¹H NMR (400 MHz, DMSO-d₆) δ: 1.94 (s, 6H) 3.24 (d, J=5.83 Hz, 2H) 4.43 (s, 2H) 6.2 (brs, 1H) 7.0 (m, 2H), 7.3-7.5 (m, 6H), 8.7 (d, 2H) 8.7 (s, 1H).

### Example 5

### N,N′-(bicyclo[2.1.1]hexane-1A-diylbis(methylene))bis(2-(4-chlorophenoxy)acetamide)

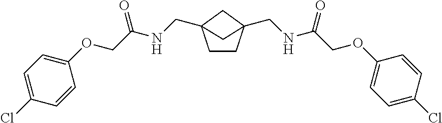 [SMILES: O=C(COc1ccc(Cl)cc1)NCC12CCC(CNC(=O)COc3ccc(Cl)cc3)(C1)C2]

### Step 1: Bicyclo[2.1.1]hexane-1,4-diyldimethanol

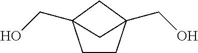

To a solution of commercially-available bicyclo[2.1.1]hexane-1,4-dicarboxylic acid (100 mg, 0.59 mmol) in tetrahydrofuran (THF) (2.0 mL) at 0° C. under an inert atmosphere of nitrogen was added LiAlH₄ (1.0 mL, 2.1 mmol, 2.0 M in THF) dropwise. A white suspension formed. The resulting reaction mixture was removed from the ice bath and allowed to warm to room temperature. The mixture was stirred for 2 h at room temperature. Following this duration, the reaction mixture was cooled back to 0° C. and cautiously quenched by the sequential addition of 100 μL water, 100 μL 5 N NaOH and 300 μL water. The resulting suspension was subsequently allowed to stir at RT for 1 h. Following this duration, the contents were filtered through Celite® and washed with EtOAc. The filtrate was diluted with water and extracted with ethyl acetate. The organic extract was dried over anhydrous MgSO₄, filtered and concentrated under reduced pressure to give the title compound as a colorless oil (63 mg, 0.44 mmol, 75% yield). ¹H NMR (400 MHz, CDCl₃) δ ppm 1.15 (dd, J=3.93, 1.90 Hz, 2H) 1.27-1.33 (br. s., 2H) 1.46 (br. s., 2H) 1.66 (t, J=1.27 Hz, 4H) 3.75 (s, 4H).

### Step 2: 2,2′-(bicyclo[2.1.1]hexane-1,4-diylbis(methylene))bis(isoindoline-1,3-dione)

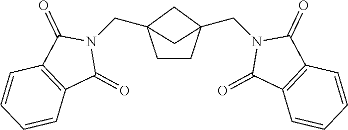 [SMILES: O=C1c2ccccc2C(=O)N1CC12CCC(CN3C(=O)c4ccccc4C3=O)(C1)C2]

To a solution of bicyclo[2.1.1]hexane-1,4-diyldimethanol (61 mg, 0.43 mmol) and phthalimide (189 mg, 1.3 mmol) in tetrahydrofuran (THF) (2.0 mL) was added polymer-bound triphenylphosphine (3 mmol/g, 429 mg, 1.3 mmol), followed by DIAD (0.250 mL, 1.287 mmol). The reaction mixture was stirred for 1 h at room temperature. Following this duration, the reaction contents were filtered and the filtrate was diluted with water and extracted with ethyl acetate. The organic extract was dried over anhydrous MgSO₄, filtered and concentrated under reduced pressure. The crude product was purified by silica gel chromatography (24 g column, 0-25% EtOAc:heptane) to give the title compound as a clear, colorless oil (157 mg, 0.39 mmol, 91% yield). LC-MS m/z 401.3 (M+H)⁺.

### Step 3: Bicyclo[2.1.1]hexane-1,4-diyldimethanamine

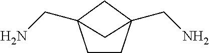

To a suspension of 2,2′-(bicyclo[2.1.1]hexane-1,4-diylbis(methylene))bis(isoindoline-1,3-dione) (155 mg, 0.387 mmol) in ethanol (3.0 mL) was added 80% hydrazine hydrate (0.24 mL, 3.9 mmol). The reaction mixture was stirred for 1 h at 50° C. The mixture was filtered and washed with ethanol. The filtrate was concentrated to give the title compound as a white solid (50 mg), which was carried forward without further purification. LC-MS m/z 141.1 (M+H)⁺.

### Step 4: N,N′-(bicyclo[2.1.1]hexane-1,4-diylbis(methylene))bis(2-(4-chlorophenoxy)acetamide)

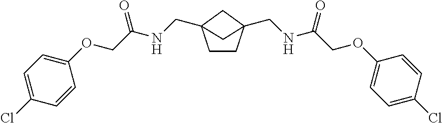 [SMILES: O=C(COc1ccc(Cl)cc1)NCC12CCC(CNC(=O)COc3ccc(Cl)cc3)(C1)C2]

To a solution of bicyclo[2.1.1]hexane-1,4-diyldimethanamine (25 mg, 0.18 mmol) in dichloromethane (DCM) (2.0 mL) was added TEA (0.15 mL, 1.1 mmol) followed by 2-(4-chlorophenoxy)acetyl chloride (0.08 mL, 0.54 mmol). The resulting reaction mixture was stirred for 30 min at room temperature. Following this duration, the reaction contents were diluted with water and extracted with dichloromethane. The organic extract was dried over anhydrous MgSO₄, filtered and concentrated under reduced pressure. The crude product was purified by silica gel chromatography (12 g Column, 0-40% EtOAc/EtOH (3:1, V:V):heptane) followed by mass-guided, reverse-phase HPLC (XSELECT CSH C18 column (150 mm×30 mm i.d. 5 μm packing diameter), 15-85% H₂O (0.1% TFA):CH₃CN (0.1% formic acid)) to give the title compound as a white solid (15 mg, 0.03 mmol, 18% yield). LC-MS m/z 477.4 (M+H)⁺. ¹H NMR (400 MHz, DMSO-d₆) δ ppm 0.90 (dd, J=3.80, 1.77 Hz, 2H) 1.13 (br. s., 2H) 1.42 (s, 4H) 3.24 (d, J=6.08 Hz, 4H) 4.51 (s, 4H) 6.95-7.00 (m, 4H) 7.32-7.38 (m, 4H) 8.05 (t, J=6.08 Hz, 2H).

### Example 6

### N,N′-(bicyclo[1.1.1]pentane-1,3-diylbis(methylene))bis(2-(4-chlorophenoxy)acetamide)

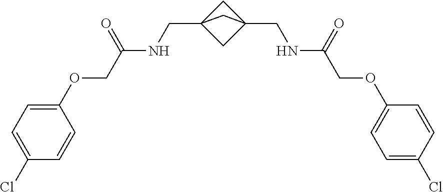 [SMILES: O=C(COc1ccc(Cl)cc1)NCC12CC(CNC(=O)COc3ccc(Cl)cc3)(C1)C2]

### Step 1: N,N′-(bicyclo[1.1.1]pentane-1,3-diylbis(methylene))bis(2-(4-chlorophenoxy)acetamide)

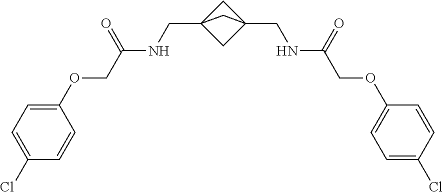 [SMILES: O=C(COc1ccc(Cl)cc1)NCC12CC(CNC(=O)COc3ccc(Cl)cc3)(C1)C2]

Prepared analogously to Example 5 starting with commercially-available bicyclo[1.1.1]pentane-1,3-dicarboxylic acid. LC-MS m/z 463.1 (M+H)⁺. ¹H NMR (400 MHz, DMSO-d₆) δ ppm 1.42 (s, 6H) 3.14-3.21 (m, 4H) 4.48-4.51 (m, 4H) 6.94-6.99 (m, 4H) 7.31-7.37 (m, 4H).

### Example 7

### 2-(4-Chlorophenoxy)-N-((3-(2-(4-chlorophenoxy)acetamido)bicyclo[1.1.1]pentan-1-yl)methyl)acetamide

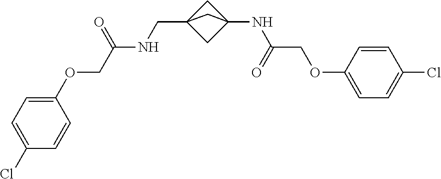 [SMILES: O=C(COc1ccc(Cl)cc1)NCC12CC(NC(=O)COc3ccc(Cl)cc3)(C1)C2]

### Step 1: tert-Butyl (3-((2-(4-chlorophenoxy)acetamido)methyl)bicyclo[1.1.1]pentan-1-yl)carbamate

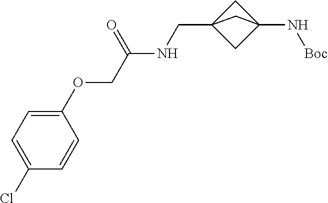 [SMILES: CC(C)(C)OC(=O)NC12CC(CNC(=O)COc3ccc(Cl)cc3)(C1)C2]

To a stirred solution of 2-(4-chlorophenoxy)acetic acid (0.26 g, 1.4 mmol) in dichloromethane (10 mL) at 0° C. was added TEA (0.4 mL, 2.8 mmol) dropwise. After 10 min at 0° C., T3P (0.84 mL, 1.4 mmol, 50% wt. in EtOAc) was added dropwise. After 5 min, commercially-available tert-butyl (3-(aminomethyl)bicyclo[1.1.1]pentan-1-yl)carbamate (0.20 g, 0.94 mmol) was added and the resulting reaction mixture was allowed to warm to RT. After 12 h, the reaction contents were concentrated under reduce pressure, and the resulting residue was quenched with 15 mL water and 15 mL saturated aqueous NaHCO₃ and stirred at RT for 30 min. The resulting solid was filtered through a sintered funnel and the solid was triturated with 10 mL diethyl ether and 10 mL n-pentane to give the title compound as an off-white solid (0.3 g, 84% yield). LC-MS m/z 325 (M+H)⁺.

### Step 2: N-((3-Aminobicyclo[1.1.1]pentan-1-yl)methyl)-2-(4-chlorophenoxy)acetamide-hydrochloride

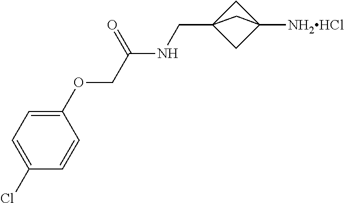

To a stirred solution of tert-butyl (3-((2-(4-chlorophenoxy)acetamido)methyl)bicyclo[1.1.1]-pentan-1-yl)carbamate (0.3 g, 0.79 mmol) in dichloromethane (15 mL) at 0° C. was added hydrochloric acid (10 mL, 4M in 1,4-dioxane) dropwise. The resulting reaction mixture was warmed to RT and allowed to stir for 12 h. Following this duration, the reaction contents were concentrated under reduced pressure and the resulting solid was triturated with diethyl ether (5 mL) and n-pentane (5 mL) and dried under vacuum to give the title compound (0.25 g), which was carried forward without further purification. LC-MS m/z 281.1 (M+H)⁺.

### Step 3: 2-(4-Chlorophenoxy)-N-((3-(2-(4-chlorophenoxy)acetamido)bicyclo[1.1.1]pentan-1-yl)methyl)acetamide

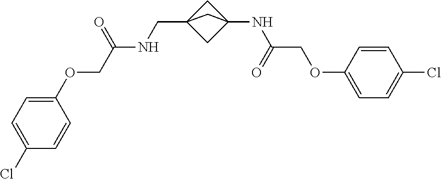 [SMILES: O=C(COc1ccc(Cl)cc1)NCC12CC(NC(=O)COc3ccc(Cl)cc3)(C1)C2]

To a stirred solution of 2-(4-chlorophenoxy)acetic acid (61 mg, 0.33 mmol) in dichloromethane (10 mL) at 0° C. was added TEA (90 μL, 0.66 mmol) dropwise. After 5 min at 0° C., T3P (0.2 mL, 0.33 mmol, 50% wt. in EtOAc) was added dropwise. After 10 min, a solution of N-((3-aminobicyclo-[1.1.1]pentan-1-yl)methyl)-2-(4-chlorophenoxy)acetamide-hydrochloride (0.20 g, 0.94 mmol) and TEA (0.1 mL) in DCM (5 mL) was added at 0° C., and the resulting reaction mixture was warmed to RT and stirred for 12 h. Following this duration, the reaction contents were concentrated under reduce pressure, and the resulting residue was dissolved in 10 mL water and 10 mL saturated aqueous NaH*CO₃ and stirred at RT for 30 min. The resulting solid was filtered through a sintered funnel and the solid was dissolved in DCM (15 mL) and washed with water. The layers were separated and the organic layer was concentrated under reduced pressure to give the title compound as a light brown solid (56 mg, 56% yield). LC-MS m/z 449.1 (M+H)⁺. ¹H NMR (400 MHz, DMSO-d₆) δ ppm 1.83 (s, 6H) 3.27 (d, J=6.08 Hz, 2H) 4.38 (s, 2H) 4.48 (s, 2H) 6.93-6.96 (m, 4H) 7.31-7.33 (m, 4H) 8.04 (t, J=6.08 Hz, 1H) 8.52 (br s, 1H).

The Compound of Example 2a in Table 1 is prepared generally according to procedures described for Examples 1 to 7 above.

TABLE 1

| | | | LCMS m/z | ¹H-NMR |
|---|---|---|---|---|
| Cmpd # | Structure | Name | [M + H]⁺ | (400 MHz) |
| 2a | 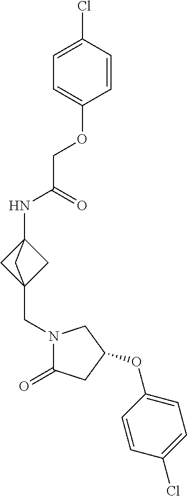 [SMILES: O=C(COc1ccc(Cl)cc1)NC12CC(CN3C[C@H](Oc4ccc(Cl)cc4)CC3=O)(C1)C2] | (R)-2-(4-chlorophenoxy)-N- (3-((4-(4-chlorophenoxy)-2- oxopyrrolidin-1- yl)methyl)bicyclo[1.1.1] pentan-1-yl)acetamide | | |

The Compound of Example 3a in Table 2 was prepared generally according to procedures described for Examples 1 to 7 above.

TABLE 2

| | | | LCMS m/z | ¹H-NMR |
|---|---|---|---|---|
| Cmpd # | Structure | Name | [M + H]⁺ | (400 MHz) |
| 3a | 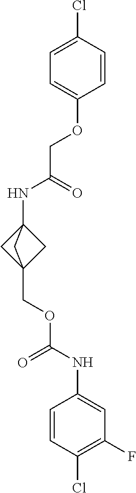 [SMILES: O=C(COc1ccc(Cl)cc1)NC12CC(COC(=O)Nc3ccc(Cl)c(F)c3)(C1)C2] | (3-(2-(4- chlorophenoxy)acetamido) bicyclo[1.1,1]pentan-1- yl)methyl (4-chloro-3- fluorophenyl)carbamate | 453.2 | ¹H NMR (400 MHz, CHLOROFORM-d) δ ppm 1.64 (s, 3 H) 2.17 (s, 6 H) 4.21- 4.35 (m, 2 H) 4.38- 4.45 (m, 2 H) 6.71- 6.83 (m, 1 H) 6.84- 6.95 (m, 3 H) 6.96- 7.08 (m, 1 H) 7.28- 7.35 (m, 4 H) 7.41- 7.51 (m, 1 H). |

The Compound of Examples 4a in Table 3 is prepared generally according to procedures described for Examples 1 to 7 above.

TABLE 3

| | | | LCMS | |
|---|---|---|---|---|
| Cmpd | | | m/z | 1H-NMR |
| # | Structure | Name | [M + H]+ | (400 MHz) |
| 4a | 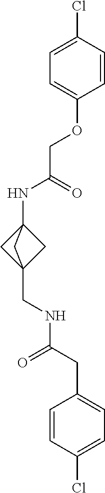 [SMILES: O=C(Cc1ccc(Cl)cc1)NCC12CC(NC(=O)COc3ccc(Cl)cc3)(C1)C2] | 2-(4-chlorophenoxy)- N-(3-((2-(4- chlorophenyl) acetamido) methyl)bicyclo [1.1.1]pentan-1- yl)acetamide | | |

The Compounds of Examples 5a to 8a in Table 4 were prepared generally according to procedures described for Examples 1 to 7 above.

TABLE 4

| | | | LCMS m/z | 1H-NMR |
|---|---|---|---|---|
| Cmpd # | Structure | Name | [M + H]+ | (400 MHz) |
| 5a | 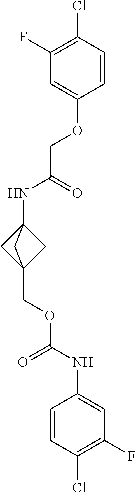 [SMILES: O=C(COc1ccc(Cl)c(F)c1)NC12CC(COC(=O)Nc3ccc(Cl)c(F)c3)(C1)C2] | (3-(2-(4-chloro-3- fluorophenoxy)acetamido) bicyclo[1.1 1]pentan-1- yl)methyl (4-chloro-3- fluorophenyl)carbamate | 471.0 | ¹H NMR (DMSO-d6) δ: 10.03 (s, 1H), 8.68 (s, 1H), 7.56 (dd, J = 12.0, 2.2 Hz, 1H), 7.49 (td, J = 8.7, 3.8 Hz, 2H), 7.26 (dd, J = 8.8, 1.5 Hz, 1H), 7.07 (dd, J = 11.4, 2.8 Hz, 1H), 6.85 (ddd, J = 8.9, 2.8, 1.2 Hz, 1H), 4.47 (s, 2H), 4.22 (s, 2H), 2.00 (s, 6H). |
| 6a | 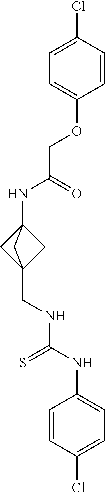 [SMILES: O=C(COc1ccc(Cl)cc1)NC12CC(CNC(=S)Nc3ccc(Cl)cc3)(C1)C2] | 2-(4-chlorophenoxy)-N-(3- ((3-(4- chlorophenyl)thioureido) methyl)bicyclo[1.1.1 ]pentan-1- yl)acetamide | 450.2 | ¹H NMR (400 MHz, MeOH-d₄) δ ppm 2.01-2.16 (m, 7 H) 3.81 (br s, 2 H) 4.46 (s, 2 H) 6.97-7.01 (m, 2 H) 7.29-7.33 (m, 2 H) 7.35-7.43 (m, 4H). |
| 7a | 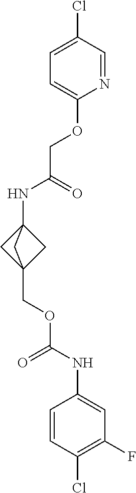 [SMILES: O=C(COc1ccc(Cl)cn1)NC12CC(COC(=O)Nc3ccc(Cl)c(F)c3)(C1)C2] | (3-(2-((5-chloropyridin-2- yl)oxy)acetamido)bicyclo [1.1.1]pentan-1-yl)methyl (4- chloro-3- fluorophenyl)carbamate | 454.0 | ¹H NMR (300 MHz, Chloroform-d) δ ppm: 8.11-8.12 (m, 1H), 7.58-7.62 (m, 1H), 7.42-7.46 (m, 1H), 7.26-7.32 (m, 1H), 6.98-7.01 (m, 1H), 6.79-6.82 (m, 1H), 6.71-6.73 (m, 2H), 4.74-4.75 (m, 2H), 4.28 (s, 2H), 2.13 (s, 6H). |
| 8a | 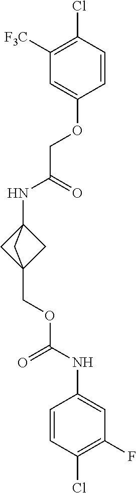 [SMILES: O=C(COc1ccc(Cl)c(C(F)(F)F)c1)NC12CC(COC(=O)Nc3ccc(Cl)c(F)c3)(C1)C2] | (3-(2-(4-chloro-3- (trifluoromethyl)phenoxy) acetamido)bicyclo[1.1.1] pentan-1-yl)methyl (4-chloro-3- fluorophenyl)carbamate | 521.1 | ¹H NMR (300 MHz, Chloroform-d) δ ppm: 7.43-7.47 (m, 2H), 7.26-7.32 (m, 2H), 6.98-7.04 (m, 2H), 6.87 (br, 1H), 6.71 (br, 1H), 4.44 (s, 2H), 4.29 (s, 2H), 2.16 (s, 6H). |

### Intermediates

### tert-butyl(3-(methylcarbamoyl)bicyclo[1.1.1]pentan-1-yl)carbamate

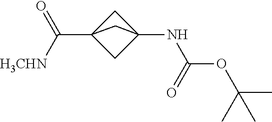

To a solution of 3-((tert-butoxycarbonyl)amino)bicyclo[1.1.1]pentane-1-carboxylic acid (300 mg, 1.320 mmol), HOBt (222 mg, 1.452 mmol) in dichloromethane (DCM) (10 mL) at room temp was added EDC (278 mg, 1.452 mmol). The reaction mixture was stirred at rt for 0.5 h. Methanamine (205 mg, 6.60 mmol) was added. The reaction mixture was stirred for 16 h at rt and then quenched with water (10 mL). The resulting solution was extracted with dichloromethane (3×10 mL) and the organic layers were combined, washed with brine (1×10 mL), dried over anhydrous sodium sulfate, filtered and concentrated under reduced pressure to afford crude product. The crude product was purified by silica gel column chromatography and eluted with ethyl acetate/petroleum ether to afford the title compound as a white solid (300 mg, 90% pure, 85% yield). LCMS m/z 241.2 (M+H)⁺.

### tert-butyl (3-((methylamino)methyl)bicyclo[1.1.1]pentan-1-yl)carbamate

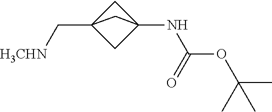

To a suspension of BH3.THF (4.49 mL, 4.49 mmol, 1M) in tetrahydrofuran (THF) (10 mL) stirred under nitrogen at 0° C. was added tert-butyl (3-(methylcarbamoyl)bicyclo[1.1.1]pentan-1-yl)carbamate (270 mg, 1.124 mmol) in tetrahydrofuran (THF) (10 mL) dropwise over 15 min. The reaction mixture was quenched with MeOH (30 mL) at 0° C. and concentrated under reduced pressure to afford the crude product. The crude product was purified by silica gel column chromatography, eluted with methanol/dichloromethane, and concentrated under reduced pressure to afford the title compound as a yellow oil (50 mg, 90% pure, 18% yield). LCMS m/z 227.2 (M+H)⁺.

### methyl 2-(4-chloro-3-fluorophenoxy)acetate

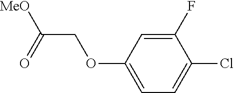 [SMILES: COC(=O)COc1ccc(Cl)c(F)c1]

To a solution of 4-chloro-3-fluorophenol (15 g, 102 mmol) in acetonitrile (400 mL) was added K₂COY (42.4 g, 307 mmol) and methyl 2-bromoacetate (16.44 g, 107 mmol). The reaction mixture was stirred for 4 h at rt. The reaction mixture was filtered and concentrated under reduced pressure, then diluted with water (150 ml) and extracted with DCM (3×200 ml). The organic layers were combined, washed with brine (200 ml), dried over anhydrous sodium sulfate, filtered and concentrated under reduced pressure to afford the title compound (22.2 g, 99% pure, 98% yield) as a yellow oil. Used without further purification. ¹HNMR (400 MHz, CDCl3) δ ppm: 7.29 (m, 1H), 6.74 (dd, J=10.4, 2.8 Hz, 1H), 6.66 (m, 1H), 4.62 (s, 2H), 3.82 (s, 3H).

### 2-(4-chloro-3-fluorophenoxy)acetic Acid

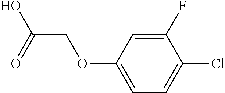 [SMILES: O=C(O)COc1ccc(Cl)c(F)c1]

To a solution of methyl 2-(4-chloro-3-fluorophenoxy)acetate (15 g, 68.6 mmol) in tetrahydrofuran (THF) (50 mL) and water (15 mL) was added lithium hydroxide hydrate (7.20 g, 172 mmol). The reaction mixture was stirred for 4 h at rt. The reaction mixture was adjusted pH to 2 with 2M HCl and extracted with EA (3×80 ml). The organic phase was collected, washed with brine (150 ml), dried over anhydrous sodium sulfate, filtered and concentrated under reduced pressure to afford the title compound (13.86 g, 96% pure, 95% yield) as a white solid. Used without further purification. ¹HNMR (300 MHz, DMSO-d6) δ ppm: 13.10 (s, 1H), 7.48 (m, 1H), 7.08 (dd, J=11.4, 2.7 Hz, 1H), 6.82 (m, 1H), 4.74 (s, 2H).

### 1-chloro-2-fluoro-4-isocyanatobenzene

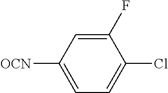

To a solution of 4-chloro-3-fluoroaniline (32 g, 220 mmol) in dichloromethane (DCM) (500 mL) at 0° C. was added 200 mL of saturated sodium bicarbonate (20.31 g, 242 mmol) followed by triphosgene (26.1 g, 88 mmol). The mixture stirred at 0° C. for 1 h. The mixture was extracted with DCM and water. The organic layer was dried over sodium sulfate and filtered. The filtrate was concentrated and treated with 50 ml hexane. The solvent was removed in vacuo to afford the title compound as a solid (39.28 g, 229 mmol, 104% yield). ¹H NMR (400 MHz, CHLOROFORM-d) δ ppm 6.95 (m, 1H) 7.32-7.49 (m, 2H).

### 4-(tert-butoxycarbonyl)-6-chloro-3,4-dihydro-2H-benzo[b][1,4]oxazine-2-carboxylic acid

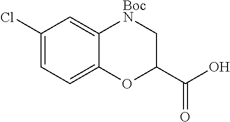 [SMILES: CC(C)(C)OC(=O)N1CC(C(=O)O)Oc2ccc(Cl)cc21]

### Step 1: 4-(tert-butyl) 2-ethyl 6-chloro-2,3-dihydro-4H-benzo[b][1,4]oxazine-2,4-dicarboxylate

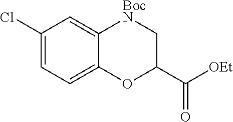 [SMILES: CCOC(=O)C1CN(C(=O)OC(C)(C)C)c2cc(Cl)ccc2O1]

To a solution of ethyl 6-chloro-3,4-dihydro-2H-benzo[b][1,4]oxazine-2-carboxylate (1.0 g, 4.14 mmol) in Tetrahydrofuran (THF) (40 ml) was added Boc-anhydride (1.921 ml, 8.28 mmol) and DMAP (0.815 g, 6.67 mmol). The reaction was stirred at room temp for 17 h. The reaction was then heated to 50° C. After 1 hour the reaction was cooled to room temp. Boc-anhydride (0.961 ml, 4.14 mmol) was added and the reaction reheated to 50° C. After 2 h, the reaction was cooled to room temp, diluted with DCM and water, and extracted with DCM. The organic layers were combined and washed with brine. The organics were then dried over MgSO₄ and filtered. To the solution was added isolute absorbant and the reaction was concentrated in vacuo for purification by flash chromatography on silica gel (40 g) (100% Heptane to 50% EA/Heptane) to afford the title compound as a clear colorless oil (1.1365 g, 3.33 mmol, 80% yield). Used without further purification. LCMS m/z 242.1 (M−100+H)⁺.

### Step 2: 4-(tert-butoxycarbonyl)-6-chloro-3,4-dihydro-2H-benzo[b][1,4]oxazine-2-carboxylic Acid

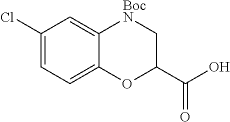 [SMILES: CC(C)(C)OC(=O)N1CC(C(=O)O)Oc2ccc(Cl)cc21]

To a solution of 4-(tert-butyl) 2-ethyl 6-chloro-2,3-dihydro-4H-benzo[b][1,4]oxazine-2,4-dicarboxylate (1.1365 g, 3.33 mmol) in tetrahydrofuran (THF) (12 ml) and water (12.00 ml) was added LiOH (0.398 g, 16.63 mmol). After 18 h at rt, the reaction was acidified to pH=2 using 1N HCl. The resulting solution was extracted with EtOAc (2×). The combined organics were washed with brine, dried over MgSO₄, filtered and concentrated in vacuo to afford the title compound as a white solid (918.2 mg, 2.93 mmol, 88% yield). Used without further purification. LCMS m/z 214.2 (M−100+H)⁺.

### 6-chloro-4-methyl-3,4-dihydro-2H-benzo[b][1,4]oxazine-2-carboxylic acid

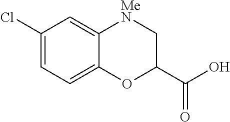 [SMILES: CN1CC(C(=O)O)Oc2ccc(Cl)cc21]

### Step 1: ethyl 6-chloro-4-methyl-3,4-dihydro-2H-benzo[b][1,4]oxazine-2-carboxylate

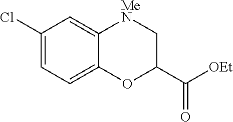 [SMILES: CCOC(=O)C1CN(C)c2cc(Cl)ccc2O1]

To a solution of ethyl 6-chloro-3,4-dihydro-2H-benzo[b][1,4]oxazine-2-carboxylate (733.6 mg, 3.04 mmol) in acetone (24.28 mL) was added K₂CO₃ (1100 mg, 7.96 mmol) and iodomethane (0.6 mL, 9.60 mmol) and the reaction was heated overnight at 55° C. The reaction was cooled to room temp. Iodomethane (2.0 mL, 32.0 mmol) was added, and the reaction was reheated to 55° C. and heated for 3 days. The reaction was cooled to rt, quenched with water, and extracted with EtOAc (2×). The combined organics were washed with brine, dried over MgSO₄, and filtered. Isolute absorbant was added and the reaction was concentrated in vacuo for purification by flash column chromatography on silica gel (40 g) (100% heptane to 40% EtOAc/heptane). The desired peak was concentrated in vacuo to afford the title compound as a clear colorless oil (664.4 mg, 2.60 mmol, 86% yield) and used without further purification. LCMS m/z 256.0 (M+H)⁺.

### Step 2: 6-chloro-4-methyl-3,4-dihydro-2H-benzo[b][1,4]oxazine-2-carboxylic Acid

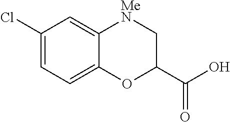 [SMILES: CN1CC(C(=O)O)Oc2ccc(Cl)cc21]

To a solution of ethyl 6-chloro-4-methyl-3,4-dihydro-2H-benzo[b][1,4]oxazine-2-carboxylate (664.4 mg, 2.60 mmol) in tetrahydrofuran (THF) (10 mL) and water (10.00 mL) was added LiOH (315 mg, 13.15 mmol). The reaction was stirred at rt overnight. The reaction was then acidified to pH=2 using 1N HCl. The resulting solution was diluted with water and extracted with EtOAc (2×). The combined organics were washed with brine (2×), dried over MgSO₄, filtered and concentrated under reduced pressure to afford the title compound as an off white solid (547.3 mg, 2.404 mmol, 93% yield). Used without further purification. LCMS m/z 228.1 (M+H)⁺.

The Compounds of Examples 8 to 21 in Table 5 are prepared generally according to procedures described for Examples 1 to 7 above.

TABLE 5

| | | | LCMS m/z | ¹H-NMR |
|---|---|---|---|---|
| Cmpd # | Structure | Name | [M + H]⁺ | (400 MHz) |
| 8 | 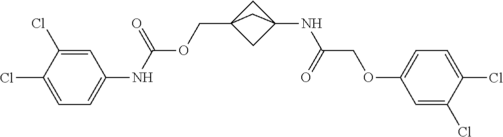 [SMILES: O=C(COc1ccc(Cl)c(Cl)c1)NC12CC(COC(=O)Nc3ccc(Cl)c(Cl)c3)(C1)C2] | (3-(2-(3,4- dichlorophenoxy) acetamido)bicyclo [1.1.1]pentan-1- yl)methyl (3,4- dichlorophenyl) carbamate | 505 [M + 3H]⁺ | ¹H NMR (400 MHz, CHLOROFORM- d) δ ppm 1.21- 1.28 (m, 1 H) 1.62 (s, 4 H) 2.14-2.22 (m, 7 H) 4.31 (s, 2 H) 4.41 (s, 2 H) 6.67-6.76 (m, 1 |
| | | | | H) 6.69-6.69 |
| | | | | (m, 1 H) 6.73- |
| | | | | 6.90 (m, 2 H) |
| | | | | 7.07 (d, J = 3.04 |
| | | | | Hz, 1 H) 7.22 (br |
| | | | | dd, J = 8.74, 1.65 |
| | | | | Hz, 1 H) 7.32- |
| | | | | 7.45 (m, 3 H) |
| | | | | 7.65 (br d, |
| | | | | J = 1.77 Hz, 1 H). |
| 9 | 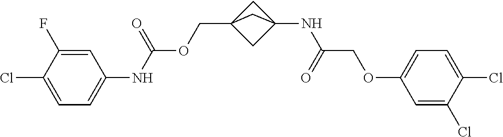 [SMILES: O=C(COc1ccc(Cl)c(Cl)c1)NC12CC(COC(=O)Nc3ccc(Cl)c(F)c3)(C1)C2] | (3-(2-(3,4- dichlorophenoxy) acetamido)bicyclo [1.1.1]pentan-1- yl)methyl (4- chloro-3- fluorophenyl) carbamate | 489.1 [M + 2H]⁺ | ¹H NMR (400 MHz, CHLOROFORM- d) δ ppm 1.62 (s, 4 H) 2.17 (s, 6 H) 4.31 (s, 2 H) 4.41 (s, 2 H) 6.74-6.88 (m, 3 H) 6.95-7.11 (m, 2 H) 7.30- |
| | | | | 7.35 (m, 1 H) |
| | | | | 7.40 (d, J = 8.87 |
| | | | | Hz, 1 H) 7.41- |
| | | | | 7.49 (m, 1 H). |
| 10 | 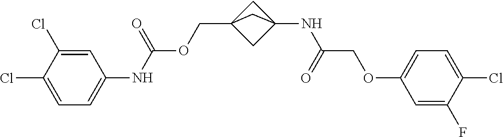 [SMILES: O=C(COc1ccc(Cl)c(F)c1)NC12CC(COC(=O)Nc3ccc(Cl)c(Cl)c3)(C1)C2] | (3-(2-(4-chloro-3- fluorophenoxy) acetamido)bicyclo [1.1.1]pentan-1- yl)methyl (3,4- dichlorophenyl) carbamate | 486.8 | ¹H NMR (400 MHz, DMSO-d₆) δ ppm 2.00 (s, 5 H) 2.09 (s, 1 H) 4.23 (s, 2 H) 4.48 (s, 2 H) 6.76-6.89 (m, 1 H) 7.08 (dd, J = 11.41, 2.79 |
| | | | | Hz, 1 H) 7.42 |
| | | | | (dd, J = 8.87, |
| | | | | 2.53 Hz, 1 H) |
| | | | | 7.44-7.62 (m, 2 |
| | | | | H) 7.79 (d, |
| | | | | J = 2.28 Hz, 1 H) |
| | | | | 8.71 (s, 1 H) |
| | | | | 10.03 (s, 1H). |
| 11 | 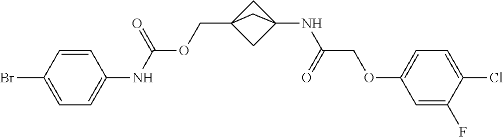 [SMILES: O=C(COc1ccc(Cl)c(F)c1)NC12CC(COC(=O)Nc3ccc(Br)cc3)(C1)C2] | (3-(2-(4-chloro-3- fluorophenoxy) acetamido)bicyclo [1.1.1]pentan-1- yl)methyl (4- bromophenyl) carbamate | 497.1 | 1H NMR (400 MHz, DMSO-d6) δ ppm 1.87- 2.08 (m, 6 H) 4.13-4.28 (m, 2 H) 4.41-4.54 (m, 2 H) 6.76- 6.91 (m, 1 H) |
| | | | | 7.01-7.13 (m, 1 |
| | | | | H) 7.38-7.59 |
| | | | | (m, 5 H) 8.70 (br |
| | | | | s, 1 H) 9.83 (br |
| | | | | s, 1 H). |
| 12 | 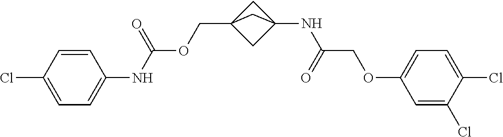 [SMILES: O=C(COc1ccc(Cl)c(Cl)c1)NC12CC(COC(=O)Nc3ccc(Cl)cc3)(C1)C2] | (3-(2-(3,4- dichlorophenoxy) acetamido)bicyclo [1.1.1]pentan-1- yl)methyl (4- chlorophenyl) carbamate | 469.2 | ¹H NMR (400 MHz, CHLOROFORM- d) δ ppm 1.21- 1.28 (m, 1 H) 1.62 (s, 4 H) 2.14-2.22 (m, 7 H) 4.31 (s, 2 H) 4.41 (s, 2 H) |
| | | | | 6.67-6.76 (m, 1 |
| | | | | H) 6.69-6.69 |
| | | | | (m, 1 H) 6.73- |
| | | | | 6.90 (m, 2 H) |
| | | | | 7.07 (d, J = 3.04 |
| | | | | Hz, 1 H) 7.22 (br |
| | | | | dd, J = 8.74, 1.65 |
| | | | | Hz, 1 H) 7.32- |
| | | | | 7.45 (m, 3 H) |
| | | | | 7.65 (br d, |
| | | | | J = 1.77 Hz, 1 H). |
| 13 | 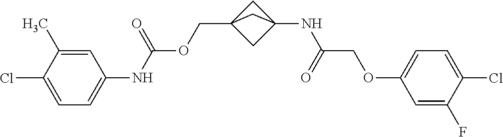 [SMILES: Cc1cc(NC(=O)OCC23CC(NC(=O)COc4ccc(Cl)c(F)c4)(C2)C3)ccc1Cl] | (3-(2-(4-chloro-3- fluorophenoxy) acetamido)bicyclo [1.1.1]pentan-1- yl)methyl (4- chloro-3- methylphenyl) carbamate | 467.1 | ¹H NMR (400 MHz, DMSO-d6) δppm 2.00 (s, 5 H) 4.15-4.23 (m, 1 H) 4.21 (s, 1 H) 4.41-4.41 (m, 1 H) 4.48 (s, 2 H) 6.49-6.57 (m, 1 H) 6.80- |
| | | | | 6.89 (m, 1 H) |
| | | | | 7.04-7.11 (m, 1 |
| | | | | H) 7.22-7.28 |
| | | | | (m, 1 H) 7.42- |
| | | | | 7.54 (m, 2 H) |
| | | | | 7.56-7.63 (m, 1 |
| | | | | H) 8.70 (s, 1 H). |
| 14 | 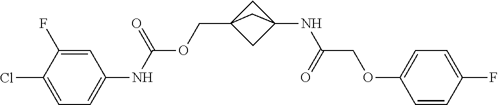 [SMILES: O=C(COc1ccc(F)cc1)NC12CC(COC(=O)Nc3ccc(Cl)c(F)c3)(C1)C2] | (3-(2-(4- fluorophenoxy) acetamido)bicyclo [1.1.1]pentan-1- yl)methyl (4- chloro-3- fluorophenyl) | 437.2 | ¹H NMR (400 MHz, DMSO-d6) δ 10.03 (s, 1H), 8.64 (s, 1H), 7.59-7.43 (m, 2H), 7.28-7.21 (m, 1H), 7.15- |
| | | carbamate | | 7.06 (m, 2H), |
| | | | | 7.00-6.90 (m, |
| | | | | 2H), 4.37 (s, |
| | | | | 2H), 4.20 (s, |
| | | | | 2H), 1.98 (s, |
| | | | | 6H). |
| 15 | 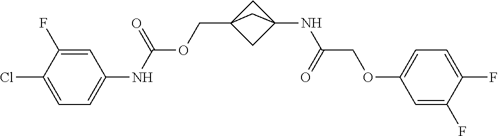 [SMILES: O=C(COc1ccc(F)c(F)c1)NC12CC(COC(=O)Nc3ccc(Cl)c(F)c3)(C1)C2] | (3-(2-(3,4- difluorophenoxy) acetamido)bicyclo [1.1.1]pentan-1- yl)methyl (4- chloro-3- fluorophenyl) carbamate | 455.2 | ¹H NMR (400 MHz, DMSO-d6) δ 10.05 (s, 1H), 8.68 (s, 1H), 7.60-7.54 (m, 1H), 7.49 (t, J = 9.0 Hz, 1H), 7.43-7.33 (m, 1H), 7.29-7.23 |
| | | | | (m, 1H), 7.14- |
| | | | | 7.05 (m, 1H), |
| | | | | 6.84-6.77 (m, |
| | | | | 1H), 4.43 (s, |
| | | | | 2H), 4.22 (s, |
| | | | | 2H), 1.99 (s, |
| | | | | 6H). |
| 16 | 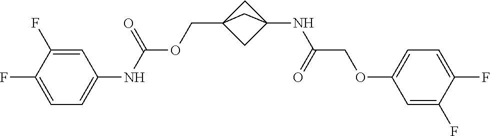 [SMILES: O=C(COc1ccc(F)c(F)c1)NC12CC(COC(=O)Nc3ccc(F)c(F)c3)(C1)C2] | (3-(2-(3,4- difluorophenoxy) acetamido)bicyclo [1.1.1]pentan-1- yl)methyl (3,4- difluorophenyl) carbamate | 439.3 | ¹H NMR (400 MHz, DMSO-d6) δ 9.91 (s, 1H), 8.67 (s, 1H), 7.60-7.50 (m, 1H), 7.41-7.31 (m, 2H), 7.24- 7.17 (m, 1H), 7.12-7.04 (m, |
| | | | | 1H), 6.82-6.76 |
| | | | | (m, 1H), 4.42 (s, |
| | | | | 2H), 4.20 (s, |
| | | | | 2H), 1.98 (s, |
| | | | | 6H). |
| 17 | 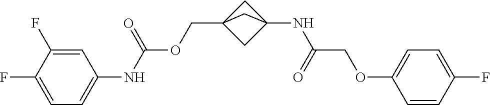 [SMILES: O=C(COc1ccc(F)cc1)NC12CC(COC(=O)Nc3ccc(F)c(F)c3)(C1)C2] | (3-(2-(4- fluorophenoxy) acetamido)bicyclo [1.1.1]pentan-1- yl)methyl (3,4- difluorophenyl) carbamate | 421.3 | ¹H NMR (400 MHz, DMSO-d6) δ 9.91 (s, 1H), 8.65 (s, 1H), 7.62-7.52 (m, 1H), 7.41-7.32 (m, 1H), 7.26- |
| | | | | 7.09 (m, 3H), |
| | | | | 7.01-6.93 (m, |
| | | | | 2H), 4.40 (s, |
| | | | | 2H), 4.21 (s, |
| | | | | 2H), 2.00 (s, |
| | | | | 6H). |
| 18 | 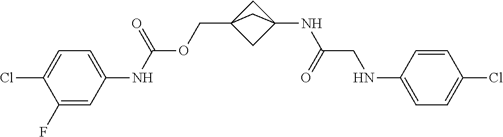 [SMILES: O=C(CNc1ccc(Cl)cc1)NC12CC(COC(=O)Nc3ccc(Cl)c(F)c3)(C1)C2] | (3-(2-((4- chlorophenyl)amino) acetamido)bicyclo [1.1.1]pentan-1-yl) methyl (4-chloro-3- fluorophenyl carbamate | 452.2 | ¹H NMR (400 MHz, DMSO-d₆) δ ppm 1.96 (s, 20 H) 2.08 (s, 1 H) 3.57 (d, J = 5.87 Hz, 7 H) 4.21 (s, 7 H) 6.02 (t, J = 5.87 Hz, 3 H) 6.50- |
| | | | | 6.56 (m, 7 H) |
| | | | | 7.07-7.14 (m, 7 |
| | | | | H) 7.26 (dd, |
| | | | | J = 8.80, 1.47 Hz, |
| | | | | 4 H) 7.45-7.60 |
| | | | | (m, 7 H) 8.50 (s, |
| | | | | 4 H) 10.04 (s, 4 |
| | | | | H). |
| 19 | 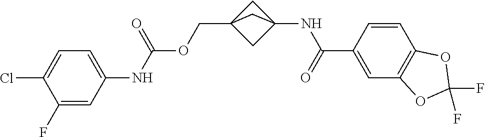 [SMILES: O=C(Nc1ccc(Cl)c(F)c1)OCC12CC(NC(=O)c3ccc4c(c3)OC(F)(F)O4)(C1)C2] | (3-(2,2- difluorobenzo[d] [1,3]dioxole-5- carboxamido) bicyclo[1.1.1] pentan-1-yl)methyl (4-chloro-3- fluorophenyl) carbamate | 469.2 | ¹H NMR (400 MHz, DMSO-d₆) δ ppm 0.84- 0.89 (m, 1 H) 1.08-1.12 (m, 1 H) 1.25 (br s, 2 H) 2.07 (s, 9 H) 2.41-2.48 (m, 1 H) 2.52-2.57 |
| | | | | (m, 1 H) 3.35- |
| | | | | 3.42 (m, 1 H) |
| | | | | 4.26 (s, 3 H) |
| | | | | 7.28 (br d, |
| | | | | J = 8.80 Hz, 2 H) |
| | | | | 7.41-7.65 (m, 6 |
| | | | | H) 7.75 (dd, |
| | | | | J = 8.56, 1.71 Hz, |
| | | | | 2 H) 7.83 (d, |
| | | | | J = 1.47 Hz, 2 H) |
| | | | | 9.08 (s, 3 H) |
| | | | | 10.08 (s, 3 H). |
| 20 | 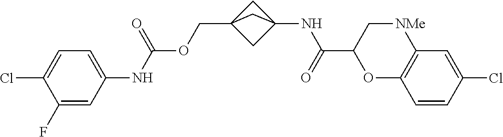 [SMILES: CN1CC(C(=O)NC23CC(COC(=O)Nc4ccc(Cl)c(F)c4)(C2)C3)Oc2ccc(Cl)cc21] | (3-(6-chloro-4-methyl- 3,4-dihydro-2H- benzo[b][1,4]oxazine- 2-carboxamido)bicyclo [1.1.1]pentan-1-yl) methyl (4-chloro-3- fluorophenyl) carbamate | 494.3 | ¹H NMR (400 MHz, CHLOROFORM- d) δ ppm 0.89- 0.93 (m, 2 H) 1.26-1.33 (m, 5 H) 2.15 (s, 10 H) 2.90 (s, 5 H) 3.29 (dd, |
| | | | | J = 11.74, 7.83 |
| | | | | Hz, 2 H) 3.54 |
| | | | | (dd, J = 11.74, |
| | | | | 2.93 Hz, 2 H) |
| | | | | 4.15 (d, J = 6.85 |
| | | | | Hz, 1 H) 4.30 (s, |
| | | | | 4 H) 4.63 (dd, |
| | | | | J = 7.58, 3.18 Hz, |
| | | | | 3 H) 6.55-6.72 |
| | | | | (m, 7 H) 6.80 (d, |
| | | | | J = 8.80 Hz, 3 H) |
| | | | | 6.93 (s, 3 H) |
| | | | | 7.01 (br d, |
| | | | | J = 8.31 Hz, 3 H) |
| | | | | 7.29-7.39 (m, 3 |
| | | | | H) 7.45 (s, 2 H) |
| | | | | 7.48 (s, 1 H). |
| 21 | 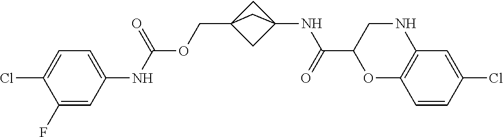 [SMILES: O=C(Nc1ccc(Cl)c(F)c1)OCC12CC(NC(=O)C3CNc4cc(Cl)ccc4O3)(C1)C2] | (3-(6-chloro-3,4- dihydro-2H- benzo[b][1,4]oxazine- 2-carboxamido)bicyclo [1.1.1]pentan-1-yl) methyl (4-chloro-3- fluorophenyl) carbamate | 480.3 | 1H NMR (400 MHz, DMSO-d6) δ ppm 1.99 (s, 5H) 2.08 (s, 1 H) 3.08-3.29 (m, 1 H) 3.40-3.49 (m, 1 H) 4.22 (s, 2 H) 4.41 (dd, J = 7.34, 2.93 Hz, 1 H) 6.18 (br s, 1 H) 6.51 (dd, |
| | | | | J = 8.31, 2.45 Hz, 1 |
| | | | | H) 6.60 (d, J = 2.45 |
| | | | | Hz, 1 H) 6.77 (d, |
| | | | | J = 8.31 Hz, 1 H) |
| | | | | 7.26 (dd, J = 8.80, |
| | | | | 1.47 Hz, 1 H) 7.45- |
| | | | | 7.60 (m, 2 H) 8.62 |
| | | | | (s, 1 H) 10.07 (s, 1 |
| | | | | H). |

### Example 22

### 2-(4-chlorophenoxy)-N-(3-((3-(3,4-dichlorophenyl)ureido)methyl)bicyclo[1.1.1]pentan-1-yl)acetamide

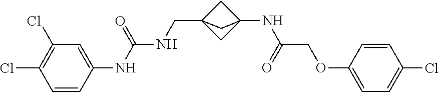 [SMILES: O=C(COc1ccc(Cl)cc1)NC12CC(CNC(=O)Nc3ccc(Cl)c(Cl)c3)(C1)C2]

### Step 1: tert-butyl (3-((3-(3,4-dichlorophenyl)ureido)methyl)bicyclo[1.1.1]pentan-1-yl)carbamate

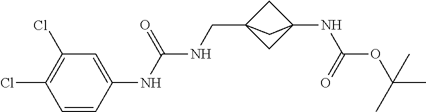 [SMILES: CC(C)(C)OC(=O)NC12CC(CNC(=O)Nc3ccc(Cl)c(Cl)c3)(C1)C2]

To a solution of tert-butyl (3-(aminomethyl)bicyclo[1.1.1]pentan-1-yl)carbamate (450 mg, 2.120 mmol) in dichloromethane (DCM) (10 mL) stirred at room temperature was added 1,2-dichloro-4-isocyanatobenzene (478 mg, 2.54 mmol) and Et₃N (0.591 mL, 4.24 mmol). The reaction mixture was stirred for 16 h at room temperature. The reaction mixture was quenched with water (10 mL). The resulting solution was extracted with ethyl acetate (3×10 mL) and the organic layers were combined, filtered and concentrated under reduced pressure to afford crude product. The sample was purified by silica gel chromatography and eluted with petroleum ether/ethyl acetate to afford the desired product as a colorless oil (750 mg, 86% pure, 76% yield). LCMS m/z 385.1 [M+CH₃CN+H−56]⁺.

### Step 2: 1-((3-aminobicyclo[1.1.1]pentan-1-yOmethyl)-3-(3,4-dichlorophenyl)urea Hydrochloride

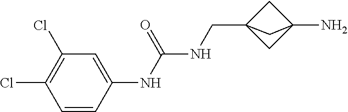 [SMILES: NC12CC(CNC(=O)Nc3ccc(Cl)c(Cl)c3)(C1)C2]

To a solution of tert-butyl (3-((3-(3,4-dichlorophenyl)ureido)methyl)bicyclo[1.1.1]pentan-1-yl)carbamate (600 mg, 1.499 mmol) in 1,4-dioxane (8 mL) stirred at room temperature was added HCl (4 mL, 132 mmol). The reaction mixture was stirred for 2 h at room temperature, then concentrated under reduced pressure to afford the desired product as a colorless oil (450 mg, 93% pure, 90% yield). Used without further purification. LCMS m/z 599.1 [2M+H]⁺.

### Step 3: 2-(4-chlorophenoxy)-N-(3-((3-(3,4-dichlorophenyl)ureido)methyObicyclo[1.1.1]pentan-1-yl)acetamide

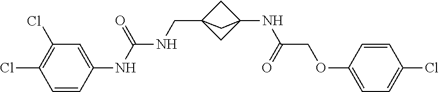 [SMILES: O=C(COc1ccc(Cl)cc1)NC12CC(CNC(=O)Nc3ccc(Cl)c(Cl)c3)(C1)C2]

To a solution of 1-((3-aminobicyclo[1.1.1]pentan-1-yl)methyl)-3-(3,4-dichlorophenyl)urea hydrochloride (220 mg, 0.657 mmol) in N,N-dimethylformamide (DMF) (8 mL) stirred at room temperature was added 2-(4-chlorophenoxy)acetic acid (205 mg, 1.099 mmol), HATU (557 mg, 1.466 mmol) and DIEA (0.384 mL, 2.199 mmol). The reaction mixture was stirred for 4 h at room temperature. The reaction mixture was quenched with water (10 mL). The resulting solution was extracted with ethyl acetate (3×10 mL) and the organic layers were combined, washed with brine (2×10 mL), dried over anhydrous sodium sulfate, filtered and concentrated under reduced pressure to afford crude product. The crude product was purified by preparative HPLC (Column: XBridge Prep OBD C18 Column 30×150 mm 5 um; Mobile Phase A: Water (10 MMOL/L NH4HCO3), Mobile Phase B: ACN; 60 mL/min; Gradient: 40% B to 70% B in 10 min; 254 nm) to provide the desired product as a white solid (136.2 mg, 99% pure, 44% yield). LCMS (ESI, m/z): 468 [M+H]⁺. ¹H NMR (300 MHz, Dimethyl sulfoxide-d6) δ ppm: 8.76 (s, 1H), 8.62 (s, 1H), 7.84-7.85 (m, 1H), 7.43-7.46 (m, 1H), 7.31-7.37 (m, 2H), 7.20-7.24 (m, 1H), 6.94-7.00 (m, 2H), 6.26-6.29 (m, 1H), 4.41 (s, 2H), 3.24-3.33 (m, 2H), 1.90 (s, 6H).

The Compounds of Examples 23 to 24 in Table 6 were prepared generally according to procedures described for Example 22 above.

TABLE 6

| | | | LCMS | |
|---|---|---|---|---|
| Cmpd | | | m/z | |
| # | Structure | Name | [M + H]⁺ | ¹H-NMR |
| 23 | 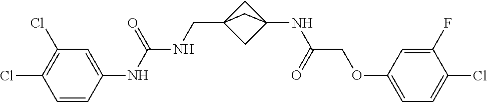 [SMILES: O=C(COc1ccc(Cl)c(F)c1)NC12CC(CNC(=O)Nc3ccc(Cl)c(Cl)c3)(C1)C2] | 2-(4-chloro-3- fluorophenoxy)-N-(3- ((3-(3,4- dichlorophenyl)ureido) methyl)bicyclo[1.1.1] pentan-1-yl)acetamide | 486.0 | (300 MHz, DMSO-d₆) δ ppm: 8.76 (s, 1H), 8.64 (s, 1H), 7.83- 7.84 (m, 1H), 7.43- 7.52 (m, 2H), 7.20- 7.24 (m, 1H), 7.04- 7.09 (m, 1H), 6.82- |
| | | | | 6.87 (m, 1H), 6.26- |
| | | | | 6.30 (m, 1H), 4.46 (s, |
| | | | | 2H), 3.24-3.33 (m, |
| | | | | 2H), 1.90 (s, 6H) |
| 24 | 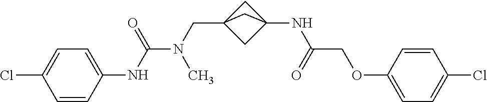 [SMILES: CN(CC12CC(NC(=O)COc3ccc(Cl)cc3)(C1)C2)C(=O)Nc1ccc(Cl)cc1] | 2-(4-chlorophenoxy)- N-(3-((3-(4- chlorophenyl)-1- methylureido)methyl) bicyclo[1.1.1 ]pentan- 1-yl)acetamide | 448.2 | (300 MHz, Methanol- d4) δ ppm: 7.34- 7.38 (m, 2H), 7.21- 7.28 (m, 4H), 6.92- 6.98 (m, 2H), 4.42 (s, 2H), 3.58 (s, 2H), 3.04 (s, 3H), 2.07 (s, 6H). |

### Example 25

### (4-(2-(4-chlorophenoxy)acetamido)bicyclo[2.1.1]hexan-1-yl)methyl (4-chloro-3-fluorophenyl)carbamate

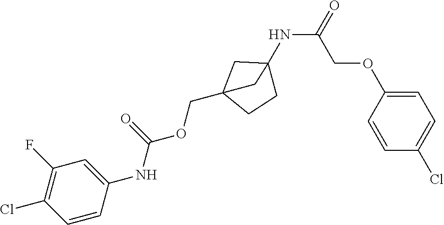 [SMILES: O=C(COc1ccc(Cl)cc1)NC12CCC(COC(=O)Nc3ccc(Cl)c(F)c3)(C1)C2]

Prepared analogously to Example 1, except using 1-chloro-2-fluoro-4-isocyanatobenzene and methyl 4-aminobicyclo[2.1.1]hexane-1-carboxylate, hydrochloride. LCMS m/z 467.2 (M+H)⁺. ¹H NMR (400 MHz, CDCl₃) δ ppm 1.59-1.71 (m, 3H) 1.96-2.08 (m, 2H) 4.31 (s, 1H) 4.42 (s, 1H) 6.79-6.94 (m, 2H) 6.96-7.11 (m, 1H) 7.25-7.35 (m, 2H).

The Compounds of Example 26 in Table 7 was prepared generally according to procedure described for Example 25 above.

TABLE 7

| | | | LCMS m/z | |
|---|---|---|---|---|
| Cmpd # | Structure | Name | [M + H]⁺ | ¹H-NMR |
| 26 | 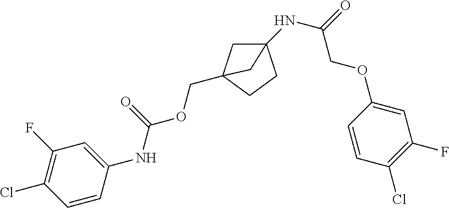 [SMILES: O=C(COc1ccc(Cl)c(F)c1)NC12CCC(COC(=O)Nc3ccc(Cl)c(F)c3)(C1)C2] | (4-(2-(4-chloro-3- fluorophenoxy)acetamido) bicyclo[2.1.1]hexan- 1-yl)methyl (4- chloro-3-fluorophenyl) carbamate | 485.2 | ¹H NMR (400 MHz, DMSO-d6) δ ppm 1.46-1.51 (m, 2H) 1.59 (brs, 2 H) 1.78- 1.88 (m, 4 H) 4.23 (s, 2 H) 4.48 (s, 2 H) 6.85 (dd, J = 8.80, 1.47 Hz, 1 H) 7.07 (dd, J = 11.25, 2.93 Hz, 1 H) 7.18-7.38 (m, 1 H) 7.45-7.60 (m, 3 H) 8.49 (s, 1 H) 10.03 (s, 1 H). |

### Assay Example 1: ATF4 Cell Based Assay

The ATF4 reporter assay measures the effect of thapsigargin-induced cellular stress on ATF4 expression. For this reporter assay, a stable cell line was created by transfecting SH-SY5Y cells with a plasmid containing the NanoLuc® luciferase gene fused to the 5′-UTR of ATF4, under the control of the CMV promoter. The ATF4 5′-UTR contains two open reading frames which mediate the cellular stress-dependent translation of the reporter gene. Clones stably expressing the reporter construct were isolated and selected based on the luminescence response to thapsigargin and inhibition of this signal by test compounds. Briefly, SH-SY5Y-ATF4-NanoLuc cells were challenged with thapsigargin to determine the stress effect with or without test compounds. Cells were propagated in DMEM/F12 growth media containing 10% FBS (Invitrogen 10999-141) and 0.5 mg/mL geneticin (Corning 30-234-CR). Aliquots of cells were cryopreserved in dialyzed FBS containing 10% DMSO.

Test compounds were prepared in neat DMSO at a concentration of 10 mM. Assay plates were prepared by adding 250 nL of compound stock solution to test wells in a 384-well white tissue culture-treated plate (Greiner 781073). For inhibition curves, compounds were diluted using a three-fold serial dilution and tested at 11 concentrations (10 μM-0.17 nM).

Aliquots of frozen cells were thawed with a 37C water bath. The cells were washed using DMEM/F12 (1:1) (1×) (Gibco 11039-021). The cells were re-suspended in the DMEM/F12 and the suspension was counted. A final suspension of 7.5e5 cells/ml was prepared.

A volume of 20 uL of cell suspension was added to compound plates (15K cells/well). Cells were incubated for 1 hour at 37° C. A volume of 5 μL of 1 uM Thapsigargin solution was added to each well, resulting in a final concentration of 200 nM. Assay plates were then incubated at 37 C overnight, typically for 19 hours.

The measurement of luciferase produced by the ATF4 constructs was measured using Nano-Glo Luciferase Assay reagent, Promega N1150. (The components of the Promega kit are: Nano-Glo® Luciferase Assay Substrate, N113C, Nano-Glo® Luciferase Assay Buffer, N1128.) The buffer is brought to room temperature, and a solution of 50:1 buffer:substrate were prepared. The cell plates were equilibrated to room temperature. A volume of 20 microliters/well of the mixed Nano-Glo reagent were dispensed into assay and control wells. The plates were read on a Viewlux plate reader.

### Biological Activity

Compounds of the invention are tested for activity against ATF4 translation in the above assay.

The compounds of Examples 1 to 7, 3a, 5a to 8a and 8 to 26 were tested generally according to the above ATF4 cell based assay and in a set of two or more experimental runs exhibited an average ATF4 pathway inhibitory activity IC50≥4 and ≤1259 nM.

The compound of Example 1 was tested generally according to the above ATF4 cell based assay and in at least one set of two or more experimental runs exhibited an average ATF4 pathway inhibitory activity IC50 of 25 nM.

The compound of Example 3 was tested generally according to the above ATF4 cell based assay and in at least one set of two or more experimental runs exhibited an average ATF4 pathway inhibitory activity IC50 of 25 nM.

The compound of Example 4 was tested generally according to the above ATF4 cell based assay and in at least one set of two or more experimental runs exhibited an average ATF4 pathway inhibitory activity IC50 of 10 nM.

The compound of Example 6a was tested generally according to the above ATF4 cell based assay and in at least one set of two or more experimental runs exhibited an average ATF4 pathway inhibitory activity IC50 of 126 nM.

The compound of Example 8a was tested generally according to the above ATF4 cell based assay and in at least one set of two or more experimental runs exhibited an average ATF4 pathway inhibitory activity IC50 of 79 nM.

The compound of Example 10 was tested generally according to the above ATF4 cell based assay and in at least one set of two or more experimental runs exhibited an average ATF4 pathway inhibitory activity IC50 of 25 nM.

The compound of Example 12 was tested generally according to the above ATF4 cell based assay and in at least one set of two or more experimental runs exhibited an average ATF4 pathway inhibitory activity IC50 of 32 nM.

The compound of Example 14 was tested generally according to the above ATF4 cell based assay and in at least one set of two or more experimental runs exhibited an average ATF4 pathway inhibitory activity IC50 of 79 nM.

The compound of Example 16 was tested generally according to the above ATF4 cell based assay and in at least one set of two or more experimental runs exhibited an average ATF4 pathway inhibitory activity IC50 of 158 nM.

The compound of Example 18 was tested generally according to the above ATF4 cell based assay and in at least one set of two or more experimental runs exhibited an average ATF4 pathway inhibitory activity IC50 of 1259 nM.

The compound of Example 19 was tested generally according to the above ATF4 cell based assay and in at least one set of two or more experimental runs exhibited an average ATF4 pathway inhibitory activity IC50 of 158 nM.

The compound of Example 22 was tested generally according to the above ATF4 cell based assay and in at least one set of two or more experimental runs exhibited an average ATF4 pathway inhibitory activity IC50 of 6 nM.

The compound of Example 23 was tested generally according to the above ATF4 cell based assay and in at least one set of two or more experimental runs exhibited an average ATF4 pathway inhibitory activity IC50 of 8 nM.

The compound of Example 26 was tested generally according to the above ATF4 cell based assay and in at least one set of two or more experimental runs exhibited an average ATF4 pathway inhibitory activity IC50 of 80 nM.

### Formulation Example 1—Capsule Composition

An oral dosage form for administering the present invention is produced by filing a standard two-piece hard gelatin capsule with the ingredients shown in Formulation Table 1, below.

Formulation Table 1

| INGREDIENTS | AMOUNTS |
|---|---|
| (3-(2-(4-Chlorophenoxy)acetamido)bicyclo[1.1.1]pentan- | |
| 1-yl)methyl (4-chlorophenyl)carbamate (Compound of | |
| Example 1) | |
| Lactose | |
| Talc | |
| Magnesium Stearate | |

### Formulation Example 2—Injectable Parenteral Composition

An injectable form for administering the present invention is produced by stirring 1.7% by weight of 4-Chlorophenyl ((3-(2-(4-chlorophenoxy)acetamido)bicyclo[1.1.1]pentan-1-yl)methyl)carbamate (Compound of Example 2) in 10% by volume propylene glycol in water.

### Formulation Example 3 Tablet Composition

The sucrose, calcium sulfate dihydrate and an ATF4 pathway inhibitor as shown in Formulation Table 2 below, are mixed and granulated with a 10% gelatin solution. The wet granules are screened, dried, mixed with the starch, talc and stearic acid, screened and compressed into a tablet.

Formulation Table 2

| INGREDIENTS | AMOUNTS |
|---|---|
| (3-(2-(4-chloro-3- | |
| fluorophenoxy)acetamido)bicyclo[1.1.1]pentan- | |
| 1-yl)methyl (4-chlorophenyl)carbamate | |
| (Compound of Example 3) | |
| calcium sulfate dihydrate | |
| Sucrose | |
| Starch | |
| Talc | |
| stearic acid | |

### REFERENCES

1. Wek R C, Jiang H-Y, Anthony T G. Coping with stress: eIF2 kinases and translational control. Biochem. Soc. Trans. 2006 February; 34(Pt I):7-11.
2. Hinnebusch A G, Lorsch J R. The mechanism of eukaryotic translation initiation: new insights and challenges. Cold Spring Harb Perspect Biol. 2012; 4(10): a011544.
3. Krishnamoorthy T, Pavitt G D, Zhang F, Dever T E, Hinnebusch A G. Tight binding of the phosphorylated alpha subunit of initiation factor 2 (eIF2alpha) to the regulatory subunits of guanine nucleotide exchange factor eIF2B is required for inhibition of translation initiation. Mol Cell Biol. 2001 August; 21(15):5018-30.
4. Hinnebusch A G. Translational regulation of GCN4 and the general amino acid control of yeast. Annu. Rev. Microbiol. 2005; 59:407-50.
5. Jackson R J, Hellen C U T, Pestova T V. The mechanism of eukaryotic translation initiation and principles of its regulation. Nat Rev Mol Cell Biol. 2010 February I; I 1(2):113-27.
6. Harding H P, Novoa I, Zhang Y, Zeng H, Wek R, Schapira M, et al. Regulated translation initiation controls stress-induced gene expression in mammalian cells. Mol. Cell. 2000 November; 6(5):1099-108.
7. Palam L R, Baird T D, Wek R C. Phosphorylation of eIF2 facilitates ribosomal bypass of an inhibitory upstream ORF to enhance CHOP translation. Journal of Biological Chemistry. 2011 April I; 286(13):10939-49.
8. Vattem K M, Wek R C. Reinitiation involving upstream ORFs regulates ATF4 mRNA translation in mammalian cells. Proc Natl Acad Sci USA. 2004 Aug. 3; 101(31):11269-74.
9. Ma Y, Brewer J W, Diehl J A, Hendershot L M. Two distinct stress signaling pathways converge upon the CHOP promoter during the mammalian unfolded protein response. J. Mol. Biol. 2002 May 17; 318(5):1351-65.
10. Pavitt G D, Ron D. New insights into translational regulation in the endoplasmic reticulum unfolded protein response. Cold Spring Harb Perspect Biol. 2012 June; 4(6): a012278.
11. Ron D, Walter P. Signal integration in the endoplasmic reticulum unfolded protein response. Nat Rev Mol Cell Biol. 2007 July; 8(7):519-29.
12. Gardner B M, Walter P. Unfolded proteins are Irel-activating ligands that directly induce the unfolded protein response. Science. 2011 Sep. 30; 333(6051):1891-4.
13. Harding H P, Zhang Y, Bertolotti A, Zeng H, Ron D. Perk is essential for translational regulation and cell survival during the unfolded protein response. Mol Cell. 2000 May; 5(5):897-904.
14. Walter P, Ron D. The unfolded protein response: from stress pathway to homeostatic regulation. Science. 2011 Nov. 25; 334(6059):1081-6.
15. Tabas I, Ron D. Integrating the mechanisms of apoptosis induced by endoplasmic reticulum stress. Nat Cell Biol. 2011 March I; 13(3):184-90.
16. Shore G C G, Papa F R F, Oakes S A S. Signaling cell death from the endoplasmic reticulum stress response. Current Opinion in Cell Biology. 2011 April I; 23(2):143-9.
17. Bi M, Naczki C, Koritzinsky M, Fels D, 174 WO 2014/144952 PC T/US2014/029568 Blais J, Hu N, Harking H, Novoa I, Varia M, Raleigh J, Scheuner D, Kaufman R J, Bell J, Ron D, Wouters B G, Koumenis C. 2005. ER stress-regulated translation increases tolerance to extreme hypoxia and promotes tumor growth. EMBO J. 24:3470-3481.
18. Bobrovnikova-Marjon E, Pytel D, Vaites L P, Singh N, Koretzky G A, Diehl J A. 2010. PERK promotes cancer cell proliferation and tumor growth by limiting oxidative DNA damage. Oncogene 29: 3881-3895.
19. Avivar-Valderas A, Bobrovnikova-Marjon E, Diehl A, Nagi C, Debnath J, Aguirre-Guiso J A 2011. PERK integrates autophagy and oxidative stress responses to promote survival during extracellular matrix detachment. Mol Cel Biol 31:3616-3629.
20. Axten J M., Medina J. R., Feng Y., Shu A., Romeril S. P. et al. 2012. Discovery of 7-methy-5(I-([3-10 (trifluoromethyl)phenyl]acetyl)-2, 3-dihydro-1H-indol-5yl)-7H-pyrrolo[2,3-d]pyrimidin-4 amine (GSK2606414), a potent and selective first-in class inhibitor of protein kinase R (PKR)-like endplasmic reticulum kinase (PERK). J. Med. Chem. 55(16):7193-7207
21. Ye J. Kumanova M., Hart L. S., Sloane K., Zhang H. et al. 2010. The GCN2-ATF4 pathway is critical for tumour cell survival and proliferation in response to nutrient deprivation. EMBO J. 29: 2082-2096.
22. Moreno J A, Radford H, Peretti D, Steinert J R, Verity N, Martin M G, Halliday M, Morgan J, Dinsdale D, Ortori C A, Barrett D A, Tsaytler P, Bertolotti A, Willis A E, Bushell M, Mallucci G R. 2012. Sustained translational repression by eIF2n-P mediates prion neurodegeneration. Nature 485:507-511.
23. Pavitt G D and Proud C G. 2009. Protein synthesis and its control in neuronal cells with a focus on vanishing white matter disease. Biochem Soc Trans 37:1298-20 1310.
24. Costa-Mattioli M. Gobert D., Harding H., Herdy B. Azzi M., Bruno M. et al, 2005. Translational control of hippocampal synaptic plasticity and memory by the eIF2n kinase GCN2. Nature 436:1166-1173.
25. Costa-Mattioli M., Gobert D., Stern E., Garnache K., Colina R I, Cuello C., Sossin W., Kaufman R., Pelletier J., Rosenblum et al. 2007. eIF2n phosphorylation bidirectionally regulates the switch from short to long term synaptic plasticity and memory. Cell 129: 195-206.
26. Zhu P. J, Huan W., Kalikulov D., Yoo J. W., Placzek A. N., Stoica L, Zhou H., Bell J. C., Frielander M. J., Krnjevic K., Noebels J. L., Costa-Mattioli M. 2011. Suppression of PKR promotes network excitability and enhanced cognition by interferon-7-mediated disinhibition. Cell 147: 1384-1396.
27. Borck G., Shin B. S., Stiller B., et al 2012. eIF2y mutation that disrupts eIF2 complex integrity links intellectual disability to impaired translation 30 initiation. Mol Cell 48:1-6.
28. Zeenko V. V., Wang C, Majumder M, Komar A. A., Snider M. D., Merrick W. C., Kaufman R. J. and Hatzoglou M. (2008). An efficient in vitro translation system from mammalian cell lacking translational inhibition caused by eIF2 phosphorylation. RNA 14: 593-602.
29. Mikami S., Masutani M., Sonenber N., Yokoyama S. And Imataka H. 175 WO 2014/144952 PC T/US2014/029568 2006. An efficient mammalian cell-free translation system supplemented with translation factors. Protein Expr. Purif. 46:348-357.

While the preferred embodiments of the invention are illustrated by the above, it is to be understood that the invention is not limited to the precise instructions herein disclosed and that the right to all modifications coming within the scope of the following claims is reserved.

1. A compound represented by the following Formula (X): 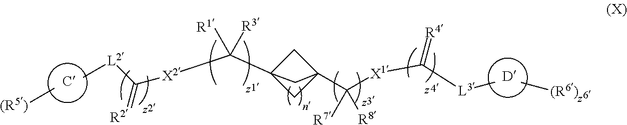
wherein:
L^{2′} is selected from: a bond, —NH—, —N(C₁₋₄alkyl)-, —N(substituted C₁₋₄alkyl)-, —O—, —S—, —S(O)—, —S(O)₂—, cycloalkyl, —O-cycloalkyl, cycloalkyl-O—, —NH-cycloalkyl, cycloalkyl-NH—, —CH₂-cycloalkyl, cycloalkyl-CH₂—, azetidinyl, —O-azetidinyl, azetidinyl-O—, —N-azetidinyl, azetidinyl-N—, substituted or unsubstituted C₁₋₆alkylene and substituted or unsubstituted C₁₋₆heteroalkylene,
or,
L^{2′} is taken together with R^{c′} to form:
heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—,
or,
L^{2′} is taken together with an R^{5′} substituent adjacent to the point of attachment of L^{2′} to C′ to form a cycloalkyl ring fused to C′, a heterocycloalkyl ring fused to C′, or a heteroaryl ring fused to C′, wherein said ring fused to C′ is optionally substituted with from 1 to 3 substituents independently selected from: F, —CH₃, —CF₃, oxo, —OH and —OCH₃;
L^{3′} is selected from: a bond, —NH—, —N(C₁₋₄alkyl)-, —N(substituted C₁₋₄alkyl)-, —O—, —S—, —S(O)—, —S(O)₂—, cycloalkyl, —O-cycloalkyl, cycloalkyl-O—, —NH— cycloalkyl, cycloalkyl-NH—, —CH₂-cycloalkyl, cycloalkyl-CH₂—, azetidinyl, —O-azetidinyl, azetidinyl-O—, —N-azetidinyl, azetidinyl-N—, substituted or unsubstituted C₁₋₆alkylene and substituted or unsubstituted C₁₋₆heteroalkylene,
or,
L^{3′} is taken together with R^{b′} to form:
heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—,
or,
L^{3′} is taken together with an R^{6′} substituent adjacent to the point of attachment of L^{3′} to D′ to form a cycloalkyl ring fused to D′, a heterocycloalkyl ring fused to D′, or heteroaryl ring fused to D′, wherein said ring fused to D′ is optionally substituted with from 1 to 3 substituents independently selected from: F, —CH₃, —CF₃, oxo, —OH and —OCH₃;
R^{1′} and R^{3′} are independently selected from: hydrogen, substituted or unsubstituted C₁₋₆alkyl, or R^{1′} and R^{3′} are taken together with the carbon to which they are attached to form a substituted or unsubstituted cycloalkyl, substituted or unsubstituted heterocycloalkyl, substituted or unsubstituted aryl, or substituted or unsubstituted heteroaryl;
R^{2′} and R^{4′} are independently NR^{a′}, O, or S;
R^{a′} is selected from: hydrogen, C₁₋₆alkyl and C₁₋₆alkyl substituted 1 to 6 times by fluoro;
R^{5′} is selected from: fluoro, chloro, bromo, iodo, —C(O)OC₁₋₄alkyl, —OH, —NH₂, —C(O)NHC₁₋₄alkyl, —OC₁₋₄alkyl, —OCH₂Ph, —C(O)Ph, —CF₃, —CN, —S(O)CH₃, —C(O)OH, —CONH₂, —NO₂, —C(O)CH₃, —C≡CH, —CH₂C≡CH, —SCH₃, —SO₃H, —SO₂NH₂, —NHC(O)NH₂, —NHC(O)H, —NHOH, —OCHF₂, —C(OH)R^{x}R^{y} (where R^{x} is selected from hydrogen, C₁₋₄alkyl, and cycloalkyl, and R^{y} is selected from C₁₋₄alkyl, and cycloalkyl), substituted or unsubstituted C₁₋₆alkyl, substituted or unsubstituted C₁₋₆heteroalkyl, substituted or unsubstituted cycloalkyl, substituted or unsubstituted heterocycloalkyl, substituted or unsubstituted aryl, or substituted or unsubstituted heteroaryl,
or,
two adjacent R^{5′} substituents can combine to form a cycloalkyl ring, a heterocycloalkyl ring, or a heteroaryl ring fused to C′,
wherein each of said rings fused to C′ is optionally substituted with from 1 to 3 substituents independently selected from: F, —CH₃, —CF₃, oxo, —OH and —OCH₃,
or,
an R^{5′} substituent adjacent to the point of attachment of L^{2′} to C′ combines with L^{2′} to form a cycloalkyl ring fused to C′, a heterocycloalkyl ring fused to C′, or a heteroaryl ring fused to C′,
wherein said ring fused to C′ is optionally substituted with from 1 to 3 substituents independently selected from: F, —CH₃, —CF₃, oxo, —OH and —OCH₃;
R^{6′} is selected from: fluoro, chloro, bromo, iodo, —C(O)OC₁₋₄alkyl, —OH, —NH₂, —C(O)NHC₁₋₄alkyl, 1-4alkyl, —OCH₂Ph, —C(O)Ph, —CF₃, —CN, —S(O)CH₃, —C(O)OH, —CONH₂, —NO₂, —C(O)CH₃, —CH₂C≡CH, —SCH₃, —SO₃H, —SO₂NH₂, —NHC(O)NH₂, —NHC(O)H, —NHOH, —OCHF₂, —C(OH)R^{x}R^{y} (where R^{x} is selected from hydrogen, C₁₋₄alkyl, and cycloalkyl, and R^{y} is selected from C₁₋₄alkyl, and cycloalkyl), substituted or unsubstituted C₁₋₆alkyl, substituted or unsubstituted C₁₋₆heteroalkyl, substituted or unsubstituted cycloalkyl, substituted or unsubstituted heterocycloalkyl, substituted or unsubstituted aryl, or substituted or unsubstituted heteroaryl,
or,
two adjacent R^{6′} substituents combine to form a cycloalkyl ring, a heterocycloalkyl ring, or heteroaryl ring fused to D′,
wherein each of said rings fused to D′ is optionally subsitituted with from 1 to 3 substituents independently selected from: F, —CH₃, —CF₃, oxo, —OH and —OCH₃,
or,
an R^{6′} substituent adjacent to the point of attachment of L^{3′} to D′ combines with L^{3′} to form a cycloalkyl ring fused to D′, a heterocycloalkyl ring fused to D′, or a heteroaryl ring fused to D,
wherein said ring fused to D′ is optionally substituted with from 1 to 3 substituents independently selected from: F, —CH₃, —CF₃, oxo, —OH and —OCH₃;
R^{7′} and R^{8′} are independently selected from: hydrogen, substituted or unsubstituted C₁₋₆alkyl, or R^{7′} and R^{8′} are taken together with the carbon to which they are attached to form a substituted or unsubstituted cycloalkyl, substituted or unsubstituted heterocycloalkyl, substituted or unsubstituted aryl, or substituted or unsubstituted heteroaryl;
C′ and D′ are independently phenyl or pyridyl;
X^{1′} is selected from: —O—, —NH—, and —NR^{b′}—;
R^{b′} is selected from: C₁₋₆alkyl, substituted C₁₋₆alkyl, cycloalkyl, and heterocycloalkyl, or R^{b′} is taken together with L^{3′} to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
X^{2′} is selected from: —O—, —NH—, and —NR^{c′}—;
R^{c′} is selected from: C₁₋₆alkyl, substituted C₁₋₆alkyl, cycloalkyl, and heterocycloalkyl, or R^{c′} is taken together with L^{2′} to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
n′ is 1 or 2;
z¹, z²′, z^{3′} and z^{4′} are independently 0 or 1; and
z^{5′} and z^{6′} are independently an integer from 0 to 5;
provided at least one of z^{1′} and z³ is 1;
or a salt thereof including a pharmaceutically acceptable salt thereof.

2. The compound according to claim 1 wherein L^{2′} is selected from: a bond, —NH—, —CH₂—O— or —O—CH₂—.

3. The compound according to claim 1 wherein L^{3′} is selected from: a bond, —NH—, —CH₂—O— or —O—CH₂—.

4. The compound according to claim 1 wherein L^{3′} is taken together with an R^{6′} substituent adjacent to the point of attachment of L^{3′} to D′ to form a heterocycloalkyl ring fused to D′, wherein said ring fused to D′ is selected from: 1,4-oxazinyl, 1,4-oxazinyl subsitituted by methyl, tetrahydropyranyl or 1,4-dioxanyl.

5. The compound according to claim 1 wherein z^{1′} is 1 and R^{1′} and R^{3′} are independently selected from: hydrogen, C₁₋₆alkyl, and C₁₋₆alkyl substituted with from 1 to 3 substituents independently selected from: —OH, —NH₂, —NHC₁₋₄alkyl, —OC₁₋₄alkyl and —OC₁₋₄alkyl substituted with —OC₁₋₃alkyl.

6. The compound according to claim 1 wherein z^{1′} is 1 and z³ is 0.

7. The compound according to claim 1 wherein R^{2′} and R^{4′} are both 0.

8. The compound according to claim 1 wherein each R^{5′} is fluoro or chloro.

9. The compound according to claim 1 wherein C′ is phenyl.

10. The compound according to claim 1 wherein D′ is phenyl or pyridyl.

11. The compound according to claim 1 wherein each of X^{1′} and X^{2′} is independently selected from: —O— and —NH—.

12. The compound according to claim 1 wherein z²′ and z^{4′} are both 1.

13. The compound according to claim 1 wherein z⁵′ and z^{6′} are independently an integer from 0 to 2.

14. The compound according to claim 1 represented by the following Formula (I): 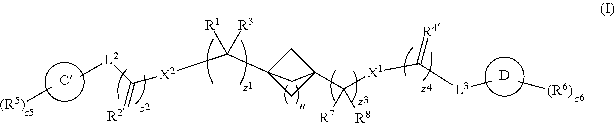
wherein:
L² is selected from: a bond, —NH—, —N(C₁₋₄alkyl)-, —N(substituted —O—, —S—, —S(O)—, —S(O)₂—, cycloalkyl, —O-cycloalkyl, cycloalkyl-O—, —NH-cycloalkyl, cycloalkyl-NH—, —CH₂-cycloalkyl, cycloalkyl-CH₂—, azetidinyl, —O-azetidinyl, azetidinyl-O—, —N-azetidinyl, azetidinyl-N—, substituted or unsubstituted C₁₋₆ alkylene and substituted or unsubstituted C₁₋₆ heteroalkylene,
or,
L² is taken together with R^{c} to form:
heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—,
or,
L² is taken together with an R⁵ substituent adjacent to the point of attachment of L² to C to form a cycloalkyl ring, a heterocycloalkyl ring, or heteroaryl ring fused to C;
L³ is selected from: a bond, —NH—, —N(C₁₋₄alkyl)-, —N(substituted —O—, —S—, —S(O)—, —S(O)₂—, cycloalkyl, —O-cycloalkyl, cycloalkyl-O—, —NH— cycloalkyl, cycloalkyl-NH—, —CH₂-cycloalkyl, cycloalkyl-CH₂—, azetidinyl, —O-azetidinyl, azetidinyl-O—, —N-azetidinyl, azetidinyl-N—, substituted or unsubstituted C₁₋₆ alkylene and substituted or unsubstituted C₁₋₆ heteroalkylene,
or,
L³ is taken together with R^{b} to form:
heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—,
or,
L³ is taken together with an R⁶ substituent adjacent to the point of attachment of L³ to D to form a cycloalkyl ring, a heterocycloalkyl ring, or heteroaryl ring fused to D;
R¹ and R³ are independently selected from: hydrogen, substituted or unsubstituted C₁₋₆ alkyl, or R¹ and R³ are taken together with the carbon to which they are attached to form a substituted or unsubstituted cycloalkyl, substituted or unsubstituted heterocycloalkyl, substituted or unsubstituted aryl, or substituted or unsubstituted heteroaryl;
R² and R⁴ are independently NR^{a}, O, or S;
R^{a} is selected from: hydrogen, C₁₋₆alkyl and C₁₋₆alkyl substituted 1 to 6 times by fluoro;
R⁵ is selected from: fluoro, chloro, bromo, iodo, —C(O)OC₁₋₄ alkyl, —OH, —NH₂, —C(O)NHC₁₋₄alkyl, —OC₁₋₄ alkyl, —OCH₂Ph, —C(O)Ph, —CF₃, —CN, —S(O)CH₃, —C(O)OH, —CONH₂, —NO₂, —C(O)CH₃, —C≡CH, —CH₂C≡CH, —SCH₃, —SO₃H, —SO₂NH₂, —NHC(O)NH₂, —NHC(O)H, —NHOH, —OCF₃, —OCHF₂, —C(OH)R^{x}R^{y} (where R^{x} is selected from hydrogen, C₁₋₄alkyl, and cycloalkyl, and R^{y} is selected from C₁₋₄alkyl, and cycloalkyl), substituted or unsubstituted C₁₋₆ alkyl, substituted or unsubstituted C₁₋₆heteroalkyl, substituted or unsubstituted cycloalkyl, substituted or unsubstituted heterocycloalkyl, substituted or unsubstituted aryl, or substituted or unsubstituted heteroaryl,
or,
two adjacent R⁵ substituents can combine to form a cycloalkyl ring, a heterocycloalkyl ring, or a heteroaryl ring fused to C,
or,
an R⁵ substituent adjacent to the point of attachment of L² to C combines with L² to form a cycloalkyl ring, a heterocycloalkyl ring, or a heteroaryl ring fused to C;
R⁶ is selected from: fluoro, chloro, bromo, iodo, —C(O)OC₁₋₄ alkyl, —OH, —NH₂, —C(O)NHC₁₋₄alkyl, —OC₁₋₄ alkyl, —OCH₂Ph, —C(O)Ph, —CF₃, —CN, —S(O)CH₃, —C(O)OH, —CONH₂, —NO₂, —C(O)CH₃, —CCH, —CH₂C CH, —SCH₃, —SO₃H, —SO₂NH₂, —NHC(O)NH₂, —NHC(O)H, —NHOH, —OCF₃, —OCHF₂, —C(OH)R^{x}R^{y} (where R^{x} is selected from hydrogen, C₁₋₄alkyl, and cycloalkyl, and R^{y} is selected from C₁₋₄alkyl, and cycloalkyl), substituted or unsubstituted C₁₋₆ alkyl, substituted or unsubstituted C₁₋₆ heteroalkyl, substituted or unsubstituted cycloalkyl, substituted or unsubstituted heterocycloalkyl, substituted or unsubstituted aryl, or substituted or unsubstituted heteroaryl,
or,
two adjacent R⁶ substituents combine to form a cycloalkyl ring, a heterocycloalkyl ring, or heteroaryl ring fused to D,
or,
an R⁶ substituent adjacent to the point of attachment of L³ to D combines with L³ to form a cycloalkyl ring, a heterocycloalkyl ring, or a heteroaryl ring fused to D;
R⁷ and R⁸ are independently selected from: hydrogen, substituted or unsubstituted C₁₋₆ alkyl, or R⁷ and R⁸ are taken together with the carbon to which they are attached to form a substituted or unsubstituted cycloalkyl, substituted or unsubstituted heterocycloalkyl, substituted or unsubstituted aryl, or substituted or unsubstituted heteroaryl;
C and D are independently phenyl or pyridyl;
X¹ is selected from: —O—, —NH—, and —NR^{b}—;
R^{b} is selected from: C₁₋₆alkyl, substituted C₁₋₆alkyl, cycloalkyl, and heterocycloalkyl, or R^{b} is taken together with L³ to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
X² is selected from: —O—, —NH—, and —NR^{c}—;
R^{c} is selected from: C₁₋₆alkyl, substituted C₁₋₆alkyl, cycloalkyl, and heterocycloalkyl, or R^{c} is taken together with L² to form: heterocycloalkyl, heterocycloalkyl-O—, heterocycloalkyl-NH—, heterocycloalkyl-CH₂—, oxoheterocycloalkyl, oxoheterocycloalkyl-O—, oxoheterocycloalkyl-N—, or oxoheterocycloalkyl-CH₂—;
n is 1 or 2;
z¹, z², z³ and z⁴are independently 0 or 1; and
z⁵ and z⁶ are independently an integer from 0 to 5;
provided at least one of z¹ and z³ is 1;
or a pharmaceutically acceptable salt thereof.

15. The compound according to claim 1 which is selected from:
(3-(2-(4-Chlorophenoxy)acetamido)bicyclo[1.1.1]pentan-1-yl)methyl (4-chlorophenyl)carbamate;
4-Chlorophenyl ((3-(2-(4-chlorophenoxy)acetamido)bicyclo[1.1.1]pentan-1-yl)methyl)carbamate;
(3-(2-(4-chloro-3-fluorophenoxy)acetamido)bicyclo[1.1.1]pentan-1-yl)methyl (4-chlorophenyl)carbamate;
2-(4-Chlorophenoxy)-N-(3-((3-(4-chlorophenyl)ureido)methyl)bicyclo[1.1.1]pentan-1-yl)acetamide;
N,N′-(bicyclo[1.1.1]hexane-1,4-diylbis(methylene))bis(2-(4-chlorophenoxy)acetamide);
N,N′-(bicyclo[1.1.1]pentane-1,3-diylbis(methylene))bis(2-(4-chlorophenoxy)acetamide);
2-(4-Chlorophenoxy)-N-((3-(2-(4-chlorophenoxy)acetamido)bicyclo[1.1.1]pentan-1-yl)methyl)acetamide;
(R)-2-(4-chlorophenoxy)-N-(3-((4-(4-chlorophenoxy)-2-oxopyrrolidin-1-yl)methyl)bicyclo[1.1.1]pentan-1-yl)acetamide;
(3-(2-(4-chlorophenoxy)acetamido)bicyclo[1.1.1]pentan-1-yl)methyl (4-chloro-3-fluorophenyl)carbamate;
2-(4-chlorophenoxy)-N-(3-((2-(4-chlorophenyl)acetamido)methyl)bicyclo[1.1.1]pentan-1-yl)acetamide;
(3-(2-(4-chloro-3-fluorophenoxy)acetamido)bicyclo[1.1.1]pentan-1-yl)methyl (4-chloro-3-fluorophenyl)carbamate;
2-(4-chlorophenoxy)-N-(3-((3-(4-chlorophenyl)thioureido)methyl)bicyclo[1.1.1]pentan-1-yl)acetamide;
(3-(2-((5-chloropyridin-2-yl)oxy)acetamido)bicyclo[1.1.1]pentan-1-yl)methyl (4-chloro-3-fluorophenyl)carbamate;
(3-(2-(4-chloro-3-(trifluoromethyl)phenoxy)acetamido)bicyclo[1.1.1]pentan-1-yl)methyl (4-chloro-3-fluorophenyl)carbamate;
(3-(2-(3,4-dichlorophenoxy)acetamido)bicyclo[1.1.1]pentan-1-yl)methyl (3,4-dichlorophenyl)carbamate;
(3-(2-(3,4-dichlorophenoxy)acetamido)bicyclo[1.1.1]pentan-1-yl)methyl (4-chloro-3-fluorophenyl)carbamate;
(3-(2-(4-chloro-3-fluorophenoxy)acetamido)bicyclo[1.1.1]pentan-1-yl)methyl (3,4-dichlorophenyl)carbamate;
(3-(2-(4-chloro-3-fluorophenoxy)acetamido)bicyclo[1.1.1]pentan-1-yl)methyl (4-bromophenyl)carbamate;
(3-(2-(3,4-dichlorophenoxy)acetamido)bicyclo[1.1.1]pentan-1-yl)methyl(4-chlorophenyl)carbamate;
(3-(2-(4-chloro-3-fluorophenoxy)acetamido)bicyclo[1.1.1]pentan-1-yl)methyl (4-chloro-3-methylphenyl)carbamate;
(3-(2-(4-fluorophenoxy)acetamido)bicyclo[1.1.1]pentan-1-yl)methyl (4-chloro-3-fluorophenyl)carbamate;
(3-(2-(3,4-difluorophenoxy)acetamido)bicyclo[1.1.1]pentan-1-yl)methyl (4-chloro-3-fluorophenyl)carbamate;
(3-(2-(3,4-difluorophenoxy)acetamido)bicyclo[1.1.1]pentan-1-yl)methyl (3,4-difluorophenyl)carbamate;
(3-(2-(4-fluorophenoxy)acetamido)bicyclo[1.1.1]pentan-1-yl)methyl (3,4-difluorophenyl)carbamate;
(3-(2-((4-chlorophenyl)amino)acetamido)bicyclo[1.1.1]pentan-1-yl)methyl (4-chloro-3-fluorophenyl)carbamate;
(3-(2,2-difluorobenzo[d][1,3]dioxole-5-carboxamido)bicyclo[1.1.1]pentan-1-yl)methyl (4-chloro-3-fluorophenyl)carbamate;
(3-(6-chloro-4-methyl-3,4-dihydro-2H-benzo[b][1,4]oxazine-2-carboxamido)bicyclo[1.1.1]pentan-1-yl)methyl (4-chloro-3-fluorophenyl)carbamate;
(3-(6-chloro-3,4-dihydro-2H-benzo[b][1,4]oxazine-2-carboxamido)bicyclo[1.1.1]pentan-1-yl)methyl (4-chloro-3-fluorophenyl)carbamate;
2-(4-chlorophenoxy)-N-(3-((3-(3,4-dichlorophenyl)ureido)methyl)bicyclo[1.1.1]pentan-1-yl)acetamide;
2-(4-chloro-3-fluorophenoxy)-N-(3-((3-(3,4-dichlorophenyl)ureido)methyl)bicyclo[1.1.1]pentan-1-yl)acetamide;
2-(4-chlorophenoxy)-N-(3-(4-chlorophenyl)-1-methylureido)methyl)bicyclo[1.1.1]pentan-1-yl)acetamide;
(4-(2-(4-chlorophenoxy)acetamido)bicyclo[2.1.1]hexan-1-yl)methyl (4-chloro-3-fluorophenyl)carbamate; and
(4-(2-(4-chloro-3-fluorophenoxy)acetamido)bicyclo[1.1.1]hexan-1-yl)methyl (4-chloro-3-fluorophenyl)carbamate;
or a pharmaceutically acceptable salt thereof.

16. A pharmaceutical composition comprising the compound according to claim 1 or a pharmaceutically acceptable salt thereof and a pharmaceutically acceptable excipient.

17. A method of inhibiting the ATF4 pathway in a human in need thereof, which comprises administering to such human a therapeutically effective amount of the compound or a pharmaceutically acceptable salt thereof as described in claim 1.

18. A method of treating a disease selected from: cancer, pre-cancerous syndromes, Alzheimer's disease, spinal cord injury, traumatic brain injury, ischemic stroke, stroke, diabetes, Parkinson disease, Huntington's disease, Creutzfeldt-Jakob Disease, prion diseases, progressive supranuclear palsy, amyotrophic lateral sclerosis, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementia, cognitive impairment, atherosclerosis, ocular diseases, neurological disorders, pain, in organ transplantation and arrhythmias, in a human in need thereof, which comprises administering to such human a therapeutically effective amount of the compound as described in claim 1 or a pharmaceutically acceptable salt thereof.

19. The method according to claim 18 wherein the disease is cancer selected from: brain (gliomas), glioblastomas, astrocytomas, glioblastoma multiforme, Bannayan-Zonana syndrome, Cowden disease, Lhermitte-Duclos disease, breast, colon, head and neck, kidney, lung, liver, melanoma, ovarian, pancreatic, adenocarcinoma, ductal adenocarcinoma, adenosquamous carcinoma, acinar cell carcinoma, glucagonoma, insulinoma, prostate, sarcoma and thyroid.

20. The method according to claim 18 wherein the disease is cancer selected from: breast cancer, inflammatory breast cancer, ductal carcinoma, lobular carcinoma, colon cancer, pancreatic cancer, insulinomas, adenocarcinoma, ductal adenocarcinoma, adenosquamous carcinoma, acinar cell carcinoma, glucagonoma, skin cancer, melanoma, metastatic melanoma, lung cancer, small cell lung cancer, non-small cell lung cancer, squamous cell carcinoma, adenocarcinoma, large cell carcinoma, brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, head and neck, kidney, liver, melanoma, ovarian, pancreatic, adenocarcinoma, ductal adenocarcinoma, adenosquamous carcinoma, acinar cell carcinoma, glucagonoma, insulinoma, prostate, sarcoma, osteosarcoma, giant cell tumor of bone, thyroid, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), neuroendocrine cancers and testicular cancer.

21. The method according to claim 18 wherein the disease is a pre-cancerous syndrome selected from: cervical intraepithelial neoplasia, monoclonal gammapathy of unknown significance (MGUS), myelodysplastic syndrome, aplastic anemia, cervical lesions, skin nevi (pre-melanoma), prostatic intraepithleial (intraductal) neoplasia (PIN), Ductal Carcinoma in situ (DCIS), colon polyps and severe hepatitis or cirrhosis.

22. The method according to claim 18 wherein the disease is an ocular disease.

23. The method according to claim 22 wherein the ocular disease is selected from: rubeosis irides; neovascular glaucoma; pterygium; vascularized glaucoma filtering blebs; conjunctival papilloma; choroidal neovascularization associated with age-related macular degeneration (AMD), myopia, prior uveitis, trauma, or idiopathic; macular edema; retinal neovascularization due to diabetes; age-related macular degeneration (AMD); macular degeneration (AMD); ocular ischemic syndrome from carotid artery disease; ophthalmic or retinal artery occlusion; sickle cell retinopathy; retinopathy of prematurity; Eale's Disease; and VonHippel-Lindau syndrome.

24. The method according to claim 23 wherein the ocular disease is selected from: age-related macular degeneration (AMD) and macular degeneration.

25. The method according to claim 18 wherein the disease is neurodegeneration.

26. A method of preventing organ damage during the transportation of organs for transplantation, which comprises adding the compound or a pharmaceutically acceptable salt thereof as described in claim 1, to a solution housing the organ during transportation.

27. A method of treating or lessening the severity of an integrated stress response associated disease in a human in need thereof, which comprises administering to such human a therapeutically effective amount of the compound or a pharmaceutically acceptable salt thereof as described in claim 1.

28. A method of treating a disease associated with phosphorylation of eIF2a in a human in need thereof, which comprises administering to such human a therapeutically effective amount of the compound or a pharmaceutically acceptable salt thereof as described in claim 1.

29. (canceled)

30. (canceled)

31. (canceled)

32. (canceled)

33. A method of treating cancer in a mammal in need thereof, which comprises: administering to such mammal a therapeutically effective amount of
a) the compound or a pharmaceutically acceptable salt thereof as described in claim 1; and
b) at least one anti-neoplastic agent.

34. The method of claim 33, wherein the at least one anti-neoplastic agent is selected from the group consisting of: anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, cell cycle signaling inhibitors, proteasome inhibitors, and inhibitors of cancer metabolism.

35. The method of claim 34, wherein the at least one anti-neoplastic agent is pazopanib.

36. A pharmaceutical combination comprising:
a) the compound as described in claim 1 or a pharmaceutically acceptable salt thereof; and
b) at least one anti-neoplastic agent.

37. (canceled)

38. A process for preparing a pharmaceutical composition containing a pharmaceutically acceptable excipient and an effective amount of a compound or a pharmaceutically acceptable salt thereof as described in claim 1, which process comprises bringing the compound or a pharmaceutically acceptable salt thereof into association with a pharmaceutically acceptable excipient.

39. A pharmaceutical composition comprising from 0.5 to 1,000 mg of the compound or pharmaceutically acceptable salt thereof as defined in claim 1, and from 0.5 to 1,000 mg of a pharmaceutically acceptable excipient.
